SEQUENCE LISTING (355 sequences)

SEQ ID NO: 1 - protein, 1464 aa, Homo sapiens
MFSFVDLRLLLLLAATALLTHGQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPVGPVGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLKGSNEIEIRAEGNSRFTYSVTVDGCTSHTGAWGKTVIEYKTTKTSRLPIIDVAPLDVGAPDQEFGFDVGPVCFL

SEQ ID NO: 2 - protein, 1487 aa, Homo sapiens
MIRLGAPQSLVLLTLLVAAVLRCQGQDVQEAGSCVQDGQRYNDKDVWKPEPCRICVCDTGTVLCDDIICEDVKDCLSPEIPFGECCPICPTDLATASGQPGPKGQKGEPGDIKDIVGPKGPPGPQGPAGEQGPRGDRGDKGEKGAPGPRGRDGEPGTPGNPGPPGPPGPPGPPGLGGNFAAQMAGGFDEKAGGAQLGVMQGPMGPMGPRGPPGPAGAPGPQGFQGNPGEPGEPGVSGPMGPRGPPGPPGKPGDDGEAGKPGKAGERGPPGPQGARGFPGTPGLPGVKGHRGYPGLDGAKGEAGAPGVKGESGSPGENGSPGPMGPRGLPGERGRTGPAGAAGARGNDGQPGPAGPPGPVGPAGGPGFPGAPGAKGEAGPTGARGPEGAQGPRGEPGTPGSPGPAGASGNPGTDGIPGAKGSAGAPGIAGAPGFPGPRGPPGPQGATGPLGPKGQTGEPGIAGFKGEQGPKGEPGPAGPQGAPGPAGEEGKRGARGEPGGVGPIGPPGERGAPGNRGFPGQDGLAGPKGAPGERGPSGLAGPKGANGDPGRPGEPGLPGARGLTGRPGDAGPQGKVGPSGAPGEDGRPGPPGPQGARGQPGVMGFPGPKGANGEPGKAGEKGLPGAPGLRGLPGKDGETGAAGPPGPAGPAGERGEQGAPGPSGFQGLPGPPGPPGEGGKPGDQGVPGEAGAPGLVGPRGERGFPGERGSPGAQGLQGPRGLPGTPGTDGPKGASGPAGPPGAQGPPGLQGMPGERGAAGIAGPKGDRGDVGEKGPEGAPGKDGGRGLTGPIGPPGPAGANGEKGEVGPPGPAGSAGARGAPGERGETGPPGPAGFAGPPGADGQPGAKGEQGEAGQKGDAGAPGPQGPSGAPGPQGPTGVTGPKGARGAQGPPGATGFPGAAGRVGPPGSNGNPGPPGPPGPSGKDGPKGARGDSGPPGRAGEPGLQGPAGPPGEKGEPGDDGPSGAEGPPGPQGLAGQRGIVGLPGQRGERGFPGLPGPSGEPGKQGAPGASGDRGPPGPVGPPGLTGPAGEPGREGSPGADGPPGRDGAAGVKGDRGETGAVGAPGAPGPPGSPGPAGPTGKQGDRGEAGAQGPMGPSGPAGARGIQGPQGPRGDKGEAGEPGERGLKGHRGFTGLQGLPGPPGPSGDQGASGPAGPSGPRGPPGPVGPSGKDGANGIPGPIGPPGPRGRSGETGPAGPPGNPGPPGPPGPPGPGIDMSAFAGLGPREKGPDPLQYMRADQAAGGLRQHDAEVDATLKSLNNQIESIRSPEGSRKNPARTCRDLKLCHPEWKSGDYWIDPNQGCTLDAMKVFCNMETGETCVYPNPANVPKKNWWSSKSKEKKHIWFGETINGGFHFSYGDDNLAPNTANVQMTFLRLLSTEGSQNITYHCKNSIAYLDEAAGNLKKALLIQGSNDVEIRAEGNSRFTYTALKDGCTKHTGKWGKTVIEYRSQKTSRLPIIDIAPMDIGGPEQEFGVDIGPVCFL

SEQ ID NO: 3 - protein, 1466 aa, Homo sapiens
MMSFVQKGSWLLLALLHPTIILAQQEAVEGGCSHLGQSYADRDVWKPEPCQICVCDSGSVLCDDIICDDQELDCPNPEIPFGECCAVCPQPPTAPTRPPNGQGPQGPKGDPGPPGIPGRNGDPGIPGQPGSPGSPGPPGICESCPTGPQNYSPQYDSYDVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPGLAGAPGLRGGAGPPGPEGGKGAAGPPGPPGAAGTPGLQGMPGERGGLGSPGPKGDKGEPGGPGADGVPGKDGPRGPTGPIGPPGPAGQPGDKGEGGAPGLPGIAGPRGSPGERGETGPPGPAGFPGAPGQNGEPGGKGERGAPGEKGEGGPPGVAGPPGGSGPAGPPGPQGVKGERGSPGGPGAAGFPGARGLPGPPGSNGNPGPPGPSGSPGKDGPPGPAGNTGAPGSPGVSGPKGDAGQPGEKGSPGAQGPPGAPGPLGIAGITGARGLAGPPGMPGPRGSPGPQGVKGESGKPGANGLSGERGPPGPQGLPGLAGTAGEPGRDGNPGSDGLPGRDGSPGGKGDRGENGSPGAPGAPGHPGPPGPVGPAGKSGDRGESGPAGPAGAPGPAGSRGAPGPQGPRGDKGETGERGAAGIKGHRGFPGNPGAPGSPGPAGQQGAIGSPGPAGPRGPVGPSGPPGKDGTSGHPGPIGPPGPRGNRGERGSEGSPGHPGQPGPPGPPGAPGPCCGGVGAAAIAGIGGEKAGGFAPYYGDEPMDFKINTDEIMTSLKSVNGQIESLISPDGSRKNPARNCRDLKFCHPELKSGEYWVDPNQGCKLDAIKVFCNMETGETCISANPLNVPRKHWWTDSSAEKKHVWFGESMDGGFQFSYGNPELPEDVLDVQLAFLRLLSSRASQNITYHCKNSIAYMDQASGNVKKALKLMGSNEGEFKAEGNSKFTYTVLEDGCTKHTGEWSKTVFEYRTRKAVRLPIVDIAPYDIGGPDQEFGVDVGPVCFL

SEQ ID NO: 4 - protein, 1669 aa, Homo sapiens
MGPRLSVWLLLLPAALLLHEEHSRAAAKGGCAGSGCGKCDCHGVKGQKGERGLPGLQGVIGFPGMQGPEGPQGPPGQKGDTGEPGLPGTKGTRGPPGASGYPGNPGLPGIPGQDGPPGPPGIPGCNGTKGERGPLGPPGLPGFAGNPGPPGLPGMKGDPGEILGHVPGMLLKGERGFPGIPGTPGPPGLPGLQGPVGPPGFTGPPGPPGPPGPPGEKGQMGLSFQGPKGDKGDQGVSGPPGVPGQAQVQEKGDFATKGEKGQKGEPGFQGMPGVGEKGEPGKPGPRGKPGKDGDKGEKGSPGFPGEPGYPGLIGRQGPQGEKGEAGPPGPPGIVIGTGPLGEKGERGYPGTPGPRGEPGPKGFPGLPGQPGPPGLPVPGQAGAPGFPGERGEKGDRGFPGTSLPGPSGRDGLPGPPGSPGPPGQPGYTNGIVECQPGPPGDQGPPGIPGQPGFIGEIGEKGQKGESCLICDIDGYRGPPGPQGPPGEIGFPGQPGAKGDRGLPGRDGVAGVPGPQGTPGLIGQPGAKGEPGEFYFDLRLKGDKGDPGFPGQPGMPGRAGSPGRDGHPGLPGPKGSPGSVGLKGERGPPGGVGFPGSRGDTGPPGPPGYGPAGPIGDKGQAGFPGGPGSPGLPGPKGEPGKIVPLPGPPGAEGLPGSPGFPGPQGDRGFPGTPGRPGLPGEKGAVGQPGIGFPGPPGPKGVDGLPGDMGPPGTPGRPGFNGLPGNPGVQGQKGEPGVGLPGLKGLPGLPGIPGTPGEKGSIGVPGVPGEHGAIGPPGLQGIRGEPGPPGLPGSVGSPGVPGIGPPGARGPPGGQGPPGLSGPPGIKGEKGFPGFPGLDMPGPKGDKGAQGLPGITGQSGLPGLPGQQGAPGIPGFPGSKGEMGVMGTPGQPGSPGPVGAPGLPGEKGDHGFPGSSGPRGDPGLKGDKGDVGLPGKPGSMDKVDMGSMKGQKGDQGEKGQIGPIGEKGSRGDPGTPGVPGKDGQAGQPGQPGPKGDPGISGTPGAPGLPGPKGSVGGMGLPGTPGEKGVPGIPGPQGSPGLPGDKGAKGEKGQAGPPGIGIPGLRGEKGDQGIAGFPGSPGEKGEKGSIGIPGMPGSPGLKGSPGSVGYPGSPGLPGEKGDKGLPGLDGIPGVKGEAGLPGTPGPTGPAGQKGEPGSDGIPGSAGEKGEPGLPGRGFPGFPGAKGDKGSKGEVGFPGLAGSPGIPGSKGEQGFMGPPGPQGQPGLPGSPGHATEGPKGDRGPQGQPGLPGLPGPMGPPGLPGIDGVKGDKGNPGWPGAPGVPGPKGDPGFQGMPGIGGSPGITGSKGDMGPPGVPGFQGPKGLPGLQGIKGDQGDQGVPGAKGLPGPPGPPGPYDIIKGEPGLPGPEGPPGLKGLQGLPGPKGQQGVTGLVGIPGPPGIPGFDGAPGQKGEMGPAGPTGPRGFPGPPGPDGLPGSMGPPGTPSVDHGFLVTRHSQTIDDPQCPSGTKILYHGYSLLYVQGNERAHGQDLGTAGSCLRKFSTMPFLFCNINNVCNFASRNDYSYWLSTPEPMPMSMAPITGENIRPFISRCAVCEAPAMVMAVHSQTIQIPPCPSGWSSLWIGYSFVMHTSAGAEGSGQALASPGSCLEEFRSAPFIECHGRGTCNYYANAYSFWLATIERSEMFKKPTPSTLKAGELRTHVSRCQVCMRRT

SEQ ID NO: 5 - protein, 1028 aa, Homo sapiens
MRAARALLPLLLQACWTAAQDEPETPRAVAFQDCPVDLFFVLDTSESVALRLKPYGALVDKVKSFTKRFIDNLRDRYYRCDRNLVWNAGALHYSDEVEIIQGLTRMPGGRDALKSSVDAVKYFGKGTYTDCAIKKGLEQLLVGGSHLKENKYLIVVTDGHPLEGYKEPCGGLEDAVNEAKHLGVKVFSVAITPDHLEPRLSIIATDHTYRRNFTAADWGQSRDAEEAISQTIDTIVDMIKNNVEQVCCSFECQPARGPPGLRGDPGFEGERGKPGLPGEKGEAGDPGRPGDLGPVGYQGMKGEKGSRGEKGSRGPKGYKGEKGKRGIDGVDGVKGEMGYPGLPGCKGSPGFDGIQGPPGPKGDPGAFGLKGEKGEPGADGEAGRPGSSGPSGDEGQPGEPGPPGEKGEAGDEGNPGPDGAPGERGGPGERGPRGTPGTRGPRGDPGEAGPQGDQGREGPVGVPGDPGEAGPIGPKGYRGDEGPPGSEGARGAPGPAGPPGDPGLMGERGEDGPAGNGTEGFPGFPGYPGNRGAPGINGTKGYPGLKGDEGEAGDPGDDNNDIAPRGVKGAKGYRGPEGPQGPPGHQGPPGPDECEILDIIMKMCSCCECKCGPIDLLFVLDSSESIGLQNFEIAKDFVVKVIDRLSRDELVKFEPGQSYAGVVQYSHSQMQEHVSLRSPSIRNVQELKEAIKSLQWMAGGTFTGEALQYTRDQLLPPSPNNRIALVITDGRSDTQRDTTPLNVLCSPGIQVVSVGIKDVFDFIPGSDQLNVISCQGLAPSQGRPGLSLVKENYAELLEDAFLKNVTAQICIDKKCPDYTCPITFSSPADITILLDGSASVGSHNFDTTKRFAKRLAERFLTAGRTDPAHDVRVAVVQYSGTGQQRPERASLQFLQNYTALASAVDAMDFINDATDVNDALGYVTRFYREASSGAAKKRLLLFSDGNSQGATPAAIEKAVQEAQRAGIEIFVVVVGRQVNEPHIRVLVTGKTAEYDVAYGESHLFRVPSYQALLRGVFHQTVSRKVALG

SEQ ID NO: 6 - protein, 3177 aa, Homo sapiens
MRKHRHLPLVAVFCLFLSGFPTTHAQQQQADVKNGAAADIIFLVDSSWTIGEEHFQLVREFLYDVVKSLAVGENDFHFALVQFNGNPHTEFLLNTYRTKQEVLSHISNMSYIGGTNQTGKGLEYIMQSHLTKAAGSRAGDGVPQVIVVLTDGHSKDGLALPSAELKSADVNVFAIGVEDADEGALKEIASEPLNMHMFNLENFTSLHDIVGNLVSCVHSSVSPERAGDTETLKDITAQDSADIIFLIDGSNNTGSVNFAVILDFLVNLLEKLPIGTQQIRVGVVQFSDEPRTMFSLDTYSTKAQVLGAVKALGFAGGELANIGLALDFVVENHFTRAGGSRVEEGVPQVLVLISAGPSSDEIRYGVVALKQASVFSFGLGAQAASRAELQHIATDDNLVFTVPEFRSFGDLQEKLLPYIVGVAQRHIVLKPPTIVTQVIEVNKRDIVFLVDGSSALGLANFNAIRDFIAKVIQRLEIGQDLIQVAVAQYADTVRPEFYFNTHPTKREVITAVRKMKPLDGSALYTGSALDFVRNNLFTSSAGYRAAEGIPKLLVLITGGKSLDEISQPAQELKRSSIMAFAIGNKGADQAELEEIAFDSSLVFIPAEFRAAPLQGMLPGLLAPLRTLSGTPEVHSNKRDIIFLLDGSANVGKTNFPYVRDFVMNLVNSLDIGNDNIRVGLVQFSDTPVTEFSLNTYQTKSDILGHLRQLQLQGGSGLNTGSALSYVYANHFTEAGGSRIREHVPQLLLLLTAGQSEDSYLQAANALTRAGILTFCVGASQANKAELEQIAFNPSLVYLMDDFSSLPALPQQLIQPLTTYVSGGVEEVPLAQPESKRDILFLFDGSANLVGQFPVVRDFLYKIIDELNVKPEGTRIAVAQYSDDVKVESRFDEHQSKPEILNLVKRMKIKTGKALNLGYALDYAQRYIFVKSAGSRIEDGVLQFLVLLVAGRSSDRVDGPASNLKQSGVVPFIFQAKNADPAELEQIVLSPAFILAAESLPKIGDLHPQIVNLLKSVHNGAPAPVSGEKDVVFLLDGSEGVRSGFPLLKEFVQRVVESLDVGQDRVRVAVVQYSDRTRPEFYLNSYMNKQDVVNAVRQLTLLGGPTPNTGAALEFVLRNILVSSAGSRITEGVPQLLIVLTADRSGDDVRNPSVVVKRGGAVPIGIGIGNADITEMQTISFIPDFAVAIPTFRQLGTVQQVISERVTQLTREELSRLQPVLQPLPSPGVGGKRDVVFLIDGSQSAGPEFQYVRTLIERLVDYLDVGFDTTRVAVIQFSDDPKVEFLLNAHSSKDEVQNAVQRLRPKGGRQINVGNALEYVSRNIFKRPLGSRIEEGVPQFLVLISSGKSDDEVDDPAVELKQFGVAPFTIARNADQEELVKISLSPEYVFSVSTFRELPSLEQKLLTPITTLTSEQIQKLLASTRYPPPAVESDAADIVFLIDSSEGVRPDGFAHIRDFVSRIVRRLNIGPSKVRVGVVQFSNDVFPEFYLKTYRSQAPVLDAIRRLRLRGGSPLNTGKALEFVARNLFVKSAGSRIEDGVPQHLVLVLGGKSQDDVSRFAQVIRSSGIVSLGVGDRNIDRTELQTITNDPRLVFTVREFRELPNIEERIMNSFGPSAATPAPPGVDTPPPSRPEKKKADIVFLLDGSINFRRDSFQEVLRFVSEIVDTVYEDGDSIQVGLVQYNSDPTDEFFLKDFSTKRQIIDAINKVVYKGGRHANTKVGLEHLRVNHFVPEAGSRLDQRVPQIAFVITGGKSVEDAQDVSLALTQRGVKVFAVGVRNIDSEEVGKIASNSATAFRVGNVQELSELSEQVLETLHDAMHETLCPGVTDAAKACNLDVILGFDGSRDQNVFVAQKGFESKVDAILNRISQMHRVSCSGGRSPTVRVSVVANTPSGPVEAFDFDEYQPEMLEKFRNMRSQHPYVLTEDTLKVYLNKFRQSSPDSVKVVIHFTDGADGDLADLHRASENLRQEGVRALILVGLERVVNLERLMHLEFGRGFMYDRPLRLNLLDLDYELAEQLDNIAEKACCGVPCKCSGQRGDRGPIGSIGPKGIPGEDGYRGYPGDEGGPGERGPPGVNGTQGFQGCPGQRGVKGSRGFPGEKGEVGEIGLDGLDGEDGDKGLPGSSGEKGNPGRRGDKGPRGEKGERGDVGIRGDPGNPGQDSQERGPKGETGDLGPMGVPGRDGVPGGPGETGKNGGFGRRGPPGAKGNKGGPGQPGFEGEQGTRGAQGPAGPAGPPGLIGEQGISGPRGSGGAAGAPGERGRTGPLGRKGEPGEPGPKGGIGNRGPRGETGDDGRDGVGSEGRRGKKGERGFPGYPGPKGNPGEPGLNGTTGPKGIRGRRGNSGPPGIVGQKGDPGYPGPAGPKGNRGDSIDQCALIQSIKDKCPCCYGPLECPVFPTELAFALDTSEGVNQDTFGRMRDVVLSIVNDLTIAESNCPRGARVAVVTYNNEVTTEIRFADSKRKSVLLDKIKNLQVALTSKQQSLETAMSFVARNTFKRVRNGFLMRKVAVFFSNTPTRASPQLREAVLKLSDAGITPLFLTRQEDRQLINALQINNTAVGHALVLPAGRDLTDFLENVLTCHVCLDICNIDPSCGFGSWRPSFRDRRAAGSDVDIDMAFILDSAETTTLFQFNEMKKYIAYLVRQLDMSPDPKASQHFARVAVVQHAPSESVDNASMPPVKVEFSLTDYGSKEKLVDFLSRGMTQLQGTRALGSAIEYTIENVFESAPNPRDLKIVVLMLTGEVPEQQLEEAQRVILQAKCKGYFFVVLGIGRKVNIKEVYTFASEPNDVFFKLVDKSTELNEEPLMRFGRLLPSFVSSENAFYLSPDIRKQCDWFQGDQPTKNLVKFGHKQVNVPNNVTSSPTSNPVTTTKPVTTTKPVTTTTKPVTTTTKPVTIINQPSVKPAAAKPAPAKPVAAKPVATKMATVRPPVAVKPATAAKPVAAKPAAVRPPAAAAAKPVATKPEVPRPQAAKPAATKPATTKPMVKMSREVQVFEITENSAKLHWERAEPPGPYFYDLTVTSAHDQSLVLKQNLTVTDRVIGGLLAGQTYHVAVVCYLRSQVRATYHGSFSTKKSQPPPPQPARSASSSTINLMVSTEPLALTETDICKLPKDEGTCRDFILKWYYDPNTKSCARFWYGGCGGNENKFGSQKECEKVCAPVLAKPGVISVMGT

SEQ ID NO: 7 - protein, 3063 aa, Homo sapiens
MRSRLPPALAALGAALLLSSIEAEVDPPSDLNFKIIDENTVHMSWAKPVDPIVGYRITVDPTTDGPTKEFTLSASTTETLLSELVPETEYVVTITSYDEVEESVPVIGQLTIQTGSSTKPVEKKPGKTEIQKCSVSAWTDLVFLVDGSWSVGRNNFKYILDFIAALVSAFDIGEEKTRVGVVQYSSDTRTEFNLNQYYQRDELLAAIKKIPYKGGNTMTGDAIDYLVKNTFTESAGARVGFPKVAIIITDGKSQDEVEIPARELRNVGVEVFSLGIKAADAKELKQIASTPSLNHVFNVANFDAIVDIQNEIISQVCSGVDEQLGELVSGEEVVEPPSNLIAMEVSSKYVKLNWNPSPSPVTGYKVILTPMTAGSRQHALSVGPQTTTLSVRDLSADTEYQISVSAMKGMTSSEPISIMEKTQPMKVQVECSRGVDIKADIVFLVDGSYSIGIANFVKVRAFLEVLVKSFEISPNRVQISLVQYSRDPHTEFTLKKFTKVEDIIEAINTFPYRGGSTNTGKAMTYVREKIFVPSKGSRSNVPKVMILITDGKSSDAFRDPAIKLRNSDVEIFAVGVKDAVRSELEAIASPPAETHVFTVEDFDAFQRISFELTQSICLRIEQELAAIKKKAYVPPKDLSFSEVTSYGFKTNWSPAGENVFSYHITYKEAAGDDEVTVVEPASSTSVVLSSLKPETLYLVNVTAEYEDGFSIPLAGEETTEEVKGAPRNLKVTDETTDSFKITWTQAPGRVLRYRIIYRPVAGGESREVTTPPNQRRRTLENLIPDTKYEVSVIPEYFSGPGTPLTGNAATEEVRGNPRDLRVSDPTTSTMKLSWSGAPGKVKQYLVTYTPVAGGETQEVTVRGDTTNTVLQGLKEGTQYALSVTALYASGAGDALFGEGTTLEERGSPQDLVTKDITDTSIGAYWTSAPGMVRGYRVSWKSLYDDVDTGEKNLPEDAIHTMIENLQPETKYRISVFATYSSGEGEPLTGDATTELSQDSKTLKVDEETENTMRVTWKPAPGKVVNYRVVYRPHGRGKQMVAKVPPTVTSTVLKRLQPQTTYDITVLPIYKMGEGKLRQGSGTTASRFKSPRNLKTSDPTMSSFRVTWEPAPGEVKGYKVTFHPTGDDRRLGELVVGPYDNTVVLEELRAGTTYKVNVFGMFDGGESSPLVGQEMTTLSDTTVMPILSSGMECLTRAEADIVLLVDGSWSIGRANFRTVRSFISRIVEVFDIGPKRVQIALAQYSGDPRTEWQLNAHRDKKSLLQAVANLPYKGGNTLTGMALNFIRQQNFRTQAGMRPRARKIGVLITDGKSQDDVEAPSKKLKDEGVELFAIGIKNADEVELKMIATDPDDTHAYNVADFESLSRIVDDLTINLCNSVKGPGDLEAPSNLVISERTHRSFRVSWTPPSDSVDRYKVEYYPVSGGKRQEFYVSRMETSTVLKDLKPETEYVVNVYSVVEDEYSEPLKGTEKTLPVPVVSLNIYDVGPTTMHVQWQPVGGATGYILSYKPVKDTEPTRPKEVRLGPTVNDMQLTDLVPNTEYAVTVQAVLHDLTSEPVTVREVTLPLPRPQDLKLRDVTHSTMNVFWEPVPGKVRKYIVRYKTPEEDVKEVEVDRSETSTSLKDLFSQTLYTVSVSAVHDEGESPPVTAQETTRPVPAPTNLKITEVTSEGFRGTWDHGASDVSLYRITWAPFGSSDKMETILNGDENTLVFENLNPNTIYEVSITAIYPDESESDDLIGSERTLPILTTQAPKSGPRNLQVYNATSNSLTVKWDPASGRVQKYRITYQPSTGEGNEQTTTIGGRQNSVVLQKLKPDTPYTITVSSLYPDGEGGRMTGRGKTKPLNTVRNLRVYDPSTSTLNVRWDHAEGNPRQYKLFYAPAAGGPEELVPIPGNTNYAILRNLQPDTSYTVTVVPVYTEGDGGRTSDTGRTLMRGLARNVQVYNPTPNSLDVRWDPAPGPVLQYRVVYSPVDGTRPSESIVVPGNTRMVHLERLIPDTLYSVNLVALYSDGEGNPSPAQGRTLPRSGPRNLRVFGETTNSLSVAWDHADGPVQQYRIIYSPTVGDPIDEYTTVPGRRNNVILQPLQPDTPYKITVIAVYEDGDGGHLTGNGRTVGLLPPQNIHISDEWYTRFRVSWDPSPSPVLGYKIVYKPVGSNEPMEAFVGEMTSYTLHNLNPSTTYDVNVYAQYDSGLSVPLTDQGTTLYLNVTDLKTYQIGWDTFCVKWSPHRAATSYRLKLSPADGTRGQEITVRGSETSHCFTGLSPDTDYGVTVFVQTPNLEGPGVSVKEHTTVKPTEAPTEPPTPPPPPTIPPARDVCKGAKADIVFLTDASWSIGDDNFNKVVKFIFNTVGGFDEISPAGIQVSFVQYSDEVKSEFKLNTYNDKALALGALQNIRYRGGNTRTGKALTFIKEKVLTWESGMRKNVPKVLVVVTDGRSQDEVKKAALVIQQSGFSVFVVGVADVDYNELANIASKPSERHVFIVDDFESFEKIEDNLITFVCETATSSCPLIYLDGYTSPGFKMLEAYNLTEKNFASVQGVSLESGSFPSYSAYRIQKNAFVNQPTADLHPNGLPPSYTIILLFRLLPETPSDPFAIWQITDRDYKPQVGVIADPSSKTLSFFNKDTRGEVQTVTFDTEEVKTLFYGSFHKVHIVVTSKSVKIYIDCYEIIEKDIKEAGNITTDGYEILGKLLKGERKSAAFQIQSFDIVCSPVWTSRDRCCDIPSRRDEGKCPAFPNSCTCTQDSVGPPGPPGPAGGPGAKGPRGERGISGAIGPPGPRGDIGPPGPQGPPGPQGPNGLSIPGEQGRQGMKGDAGEPGLPGRTGTPGLPGPPGPMGPPGDRGFTGKDGAMGPRGPPGPPGSPGSPGVTGPSGKPGKPGDHGRPGPSGLKGEKGDRGDIASQNMMRAVARQVCEQLISGQMNRFNQMLNQIPNDYQSSRNQPGPPGPPGPPGSAGARGEPGPGGRPGFPGTPGMQGPPGERGLPGEKGERGTGSSGPRGLPGPPGPQGESRTGPPGSTGSRGPPGPPGRPGNSGIRGPPGPPGYCDSSQCASIPYNGQGYPGSG

SEQ ID NO: 8 - protein, 1796 aa, Homo sapiens
MKIFQRKMRYWLLPPFLAIVYFCTIVQGQVAPPTRLRYNVISHDSIQISWKAPRGKFGGYKLLVTPTSGGKTNQLNLQNTATKAIIQGLMPDQNYTVQIIAYNKDKESKPAQGQFRIKDLEKRKDPKPRVKVVDRGNGSRPSSPEEVKFVCQTPAIADIVILVDGSWSIGRFNFRLVRHFLENLVTAFDVGSEKTRIGLAQYSGDPRIEWHLNAFSTKDEVIEAVRNLPYKGGNTLTGLALNYIFENSFKPEAGSRTGVSKIGILITDGKSQDDIIPPSRNLRESGVELFAIGVKNADVNELQEIASEPDSTHVYNVAEFDLMHTVVESLTRTLCSRVEEQDREIKASAHAITGPPTELITSEVTARSFMVNWTHAPGNVEKYRVVYYPTRGGKPDEVVVDGTVSSTVLKNLMSLTEYQIAVFAIYAHTASEGLRGTETTLALPMASDLLLYDVTENSMRVKWDAVPGASGYLILYAPLTEGLAGDEKEMKIGETHTDIELSGLLPNTEYTVTVYAMFGEEASDPVTGQETTLALSPPRNLRISNVGSNSARLTWDPTSRQINGYRIVYNNADGTEINEVEVDPITTFPLKGLTPLTEYTIAIFSIYDEGQSEPLTGVFTTEEVPAQQYLEIDEVTTDSFRVTWHPLSADEGLHKLMWIPVYGGKTEEVVLKEEQDSHVIEGLEPGTEYEVSLLAVLDDGSESEVVTAVGTTLDSFWTEPATTIVPTTSVTSVFQTGIRNLVVGDETTSSLRVKWDISDSDVQQFRVTYMTAQGDPEEEVIGTVMVPGSQNNLLLKPLLPDTEYKVTVTPIYTDGEGVSVSAPGKTLPSSGPQNLRVSEEWYNRLRITWDPPSSPVKGYRIVYKPVSVPGPTLETFVGADINTILITNLLSGMDYNVKIFASQASGFSDALTGMVKTLFLGVTNLQAKHVEMTSLCAHWQVHRHATAYRVVIESLQDRQKQESTVGGGTTRHCFYGLQPDSEYKISVYTKLQEIEGPSVSIMEKTQSLPTRPPTFPPTIPPAKEVCKAAKADLVFMVDGSWSIGDENFNKIISFLYSTVGALNKIGTDGTQVAMVQFTDDPRTEFKLNAYKTKETLLDAIKHISYKGGNTKTGKAIKYVRDTLFTAESGTRRGIPKVIVVITDGRSQDDVNKISREMQLDGYSIFAIGVADADYSELVSIGSKPSARHVFFVDDFDAFKKIEDELITFVCETASATCPVVHKDGIDLAGFKMMEMFGLVEKDFSSVEGVSMEPGTFNVFPCYQLHKDALVSQPTRYLHPEGLPSDYTISFLFRILPDTPQEPFALWEILNKNSDPLVGVILDNGGKTLTYFNYDQSGDFQTVTFEGPEIRKIFYGSFHKLHIVVSETLVKVVIDCKQVGEKAMNASANITSDGVEVLGKMVRSRGPGGNSAPFQLQMFDIVCSTSWANTDKCCELPGLRDDESCPDLPHSCSCSETNEVALGPAGPPGGPGLRGPKGQQGEPGPKGPDGPRGEIGLPGPQGPPGPQGPSGLSIQGMPGMPGEKGEKGDTGLPGPQGIPGGVGSPGRDGSPGQRGLPGKDGSSGPPGPPGPIGIPGTPGVPGITGSMGPQGALGPPGVPGAKGERGERGDLQSQAMVRSVARQVCEQLIQSHMARYTAILNQIPSHSSSIRTVQGPPGEPGRPGSPGAPGEQGPPGTPGFPGNAGVPGTPGERGLTGIKGEKGNPGVGTQGPRGPPGPAGPSGESRPGSPGPPGSPGPRGPPGHLGVPGPQGPSGQPGYCDPSSCSAYGVRAPHPDQPEFTPVQDELEAMELWGPGV

SEQ ID NO: 9 - protein, 1388 aa, Homo sapiens
MAPRRNNGQCWCLLMLLSVSTPLPAVTQTRGATETASQGHLDLTQLIGVPLPSSVSFVTGYGGFPAYSFGPGANVGRPARTLIPSTFFRDFAISVVVKPSSTRGGVLFAITDAFQKVIYLGLRLSGVEDGHQRIILYYTEPGSHVSQEAAAFSVPVMTHRWNRFAMIVQGEEVTLLVNCEEHSRIPFQRSSQALAFESSAGIFMGNAGATGLERFTGSLQQLTVHPDPRTPEELCDPEESSASGETSGLQEADGVAEILEAVTYTQASPKEAKVEPINTPPTPSSPFEDMELSGEPVPEGTLETTNMSIIQHSSPKQGSGEILNDTLEGVHSVDGDPITDSGSGAGAFLDIAEEKNLAATAAGLAEVPISTAGEAEASSVPTGGPTLSMSTENPEEGVTPGPDNEERLAATAAGEAEALASMPGEVEASGVAPGELDLSMSAQSLGEEATVGPSSEDSLTTAAAATEVSLSTFEDEEASGVPTDGLAPLTATMAPERAVTSGPGDEEDLAAATTEEPLITAGGEESGSPPPDGPPLPLPTVAPERWITPAQREHVGMKGQAGPKGEKGDAGEELPGPPEPSGPVGPTAGAEAEGSGLGWGSDVGSGSGDLVGSEQLLRGPPGPPGPPGLPGIPGKPGTDVFMGPPGSPGEDGPAGEPGPPGPEGQPGVDGATGLPGMKGEKGARGPNGSVGEKGDPGNRGLPGPPGKKGQAGPPGVMGPPGPPGPPGPPGPGCTMGLGFEDTEGSGSTQLLNEPKLSRPTAAIGLKGEKGDRGPKGERGMDGASIVGPPGPRGPPGHIKVLSNSLINITHGFMNFSDIPELVGPPGPDGLPGLPGFPGPRGPKGDTGLPGFPGLKGEQGEKGEPGAILTEDIPLERLMGKKGEPGMHGAPGPMGPKGPPGHKGEFGLPGRPGRPGLNGLKGTKGDPGVIMQGPPGLPGPPGPPGPPGAVINIKGAIFPIPVRPHCKMPVDTAHPGSPELITFHGVKGEKGSWGLPGSKGEKGDQGAQGPPGPPLDLAYLRHFLNNLKGENGDKGFKGEKGEKGDINGSFLMSGPPGLPGNPGPAGQKGETVVGPQGPPGAPGLPGPPGFGRPGDPGPPGPPGPPGPPAILGAAVALPGPPGPPGQPGLPGSRNLVTAFSNMDDMLQKAHLVIEGTFIYLRDSTEFFIRVRDGWKKLQLGELIPIPADSPPPPALSSNPHQLLPPPNPISSANYEKPALHLAALNMPFSGDIRADFQCFKQARAAGLLSTYRAFLSSHLQDLSTIVRKAERYSLPIVNLKGQVLFNNWDSIFSGHGGQFNMHIPIYSFDGRDIMTDPSWPQKVIWHGSSPHGVRLVDNYCEAWRTADTAVTGLASPLSTGKILDQKAYSCANRLIVLCIENSFMTDARK

SEQ ID NO: 10 - protein, 1751 aa, Homo sapiens
MAPYPCGCHILLLLFCCLAAARANLLNLNWLWFNNEDTSHAATTIPEPQGPLPVQPTADTTTHVTPRNGSTEPATAPGSPEPPSELLEDGQDTPTSAESPDAPEENIAGVGAEILNVAKGIRSFVQLWNDTVPTESLARAETLVLETPVGPLALAGPSSTPQENGTTLWPSRGIPSSPGAHTTEAGTLPAPTPSPPSLGRPWAPLTGPSVPPPSSGRASLSSLLGGAPPWGSLQDPDSQGLSPAAAAPSQQLQRPDVRLRTPLLHPLVMGSLGKHAAPSAFSSGLPGALSQVAVTTLTRDSGAWVSHVANSVGPGLANNSALLGADPEAPAGRCLPLPPSLPVCGHLGISRFWLPNHLHHESGEQVRAGARAWGGLLQTHCHPFLAWFFCLLLVPPCGSVPPPAPPPCCQFCEALQDACWSRLGGGRLPVACASLPTQEDGYCVLIGPAAERISEEVGLLQLLGDPPPQQVTQTDDPDVGLAYVFGPDANSGQVARYHFPSLFFRDFSLLFHIRPATEGPGVLFAITDSAQAMVLLGVKLSGVQDGHQDISLLYTEPGAGQTHTAASFRLPAFVGQWTHLALSVAGGFVALYVDCEEFQRMPLARSSRGLELEPGAGLFVAQAGGADPDKFQGVIAELKVRRDPQVSPMHCLDEEGDDSDGASGDSGSGLGDARELLREETGAALKPRLPAPPPVTTPPLAGGSSTEDSRSEEVEEQTTVASLGAQTLPGSDSVSTWDGSVRTPGGRVKEGGLKGQKGEPGVPGPPGRAGPPGSPCLPGPPGLPCPVSPLGPAGPALQTVPGPQGPPGPPGRDGTPGRDGEPGDPGEDGKPGDTGPQGFPGTPGDVGPKGDKGDPGVGERGPPGPQGPPGPPGPSFRHDKLTFIDMEGSGFGGDLEALRGPRGFPGPPGPPGVPGLPGEPGRFGVNSSDVPGPAGLPGVPGREGPPGFPGLPGPPGPPGREGPPGRTGQKGSLGEAGAPGHKGSKGAPGPAGARGESGLAGAPGPAGPPGPPGPPGPPGPGLPAGFDDMEGSGGPFWSTARSADGPQGPPGLPGLKGDPGVPGLPGAKGEVGADGVPGFPGLPGREGIAGPQGPKGDRGSRGEKGDPGKDGVGQPGLPGPPGPPGPVVYVSEQDGSVLSVPGPEGRPGFAGFPGPAGPKGNLGSKGERGSPGPKGEKGEPGSIFSPDGGALGPAQKGAKGEPGFRGPPGPYGRPGYKGEIGFPGRPGRPGMNGLKGEKGEPGDASLGFGMRGMPGPPGPPGPPGPPGTPVYDSNVFAESSRPGPPGLPGNQGPPGPKGAKGEVGPPGPPGQFPFDFLQLEAEMKGEKGDRGDAGQKGERGEPGGGGFFGSSLPGPPGPPGPRGYPGIPGPKGESIRGQPGPPGPQGPPGIGYEGRQGPPGPPGPPGPPSFPGPHRQTISVPGPPGPPGPPGPPGTMGASSGVRLWATRQAMLGQVHEVPEGWLIFVAEQEELYVRVQNGFRKVQLEARTPLPRGTDNEVAALQPPVVQLHDSNPYPRREHPHPTARPWRADDILASPPRLPEPQPYPGAPHHSSYVHLRPARPTSPPAHSHRDFQPVLHLVALNSPLSGGMRGIRGADFQCFQQARAVGLAGTFRAFLSSRLQDLYSIVRRADRAAVPIVNLKDELLFPSWEALFSGSEGPLKPGARIFSFDGKDVLRHPTWPQKSVWHGSDPNGRRLTESYCETWRTEAPSATGQASSLLGGRLLGQSAASCHHAYIVLCIENSFMTASK

SEQ ID NO: 11 - protein, 1142 aa, Homo sapiens
MRLTGPWKLWLWMSIFLLPASTSVTVRDKTEESCPILRIEGHQLTYDNINKLEVSGFDLGDSFSLRRAFCESDKTCFKLGSALLIRDTIKIFPKGLPEEYSVAAMFRVRRNAKKERWFLWQVLNQQNIPQISIVVDGGKKVVEFMFQATEGDVLNYIFRNRELRPLFDRQWHKLGISIQSQVISLYMDCNLIARRQTDEKDTVDFHGRTVIATRASDGKPVDIELHQLKIYCSANLIAQETCCEISDTKCPEQDGFGNIASSWVTAHASKMSSYLPAKQELKDQCQCIPNKGEAGLPGAPGSPGQKGHKGEPGENGLHGAPGFPGQKGEQGFEGSKGETGEKGEQGEKGDPALAGLNGENGLKGDLGPHGPPGPKGEKGDTGPPGPPALPGSLGIQGPQGPPGKEGQRGRRGKTGPPGKPGPPGPPGPPGIQGIHQTLGGYYNKDNKGNDEHEAGGLKGDKGETGLPGFPGSVGPKGQKGEPGEPFTKGEKGDRGEPGVIGSQGVKGEPGDPGPPGLIGSPGLKGQQGSAGSMGPRGPPGDVGLPGEHGIPGKQGIKGEKGDPGGIIGPPGLPGPKGEAGPPGKSLPGEPGLDGNPGAPGPRGPKGERGLPGVHGSPGDIGPQGIGIPGRTGAQGPAGEPGIQGPRGLPGLPGTPGTPGNDGVPGRDGKPGLPGPPGDPIALPLLGDIGALLKNFCGNCQASVPGLKSNKGEEGGAGEPGKYDSMARKGDIGPRGPPGIPGREGPKGSKGERGYPGIPGEKGDEGLQGIPGIPGAPGPTGPPGLMGRTGHPGPTGAKGEKGSDGPPGKPGPPGPPGIPFNERNGMSSLYKIKGGVNVPSYPGPPGPPGPKGDPGPVGEPGAMGLPGLEGFPGVKGDRGPAGPPGIAGMSGKPGAPGPPGVPGEPGERGPVGDIGFPGPEGPSGKPGINGKDGIPGAQGIMGKPGDRGPKGERGDQGIPGDRGSQGERGKPGLTGMKGAIGPMGPPGNKGSMGSPGHQGPPGSPGIPGIPADAVSFEEIKKYINQEVLRIFEERMAVFLSQLKLPAAMLAAQAYGRPGPPGKDGLPGPPGDPGPQGYRGQKGERGEPGIGLPGSPGLPGTSALGLPGSPGAPGPQGPPGPSGRCNPEDCLYPVSHAHQRTGGN

SEQ ID NO: 12 - protein, 1366 aa, Homo sapiens
MLSFVDTRTLLLLAVTLCLATCQSLQEETVRKGPAGDRGPRGERGPPGPPGRDGEDGPTGPPGPPGPPGPPGLGGNFAAQYDGKGVGLGPGPMGLMGPRGPPGAAGAPGPQGFQGPAGEPGEPGQTGPAGARGPAGPPGKAGEDGHPGKPGRPGERGVVGPQGARGFPGTPGLPGFKGIRGHNGLDGLKGQPGAPGVKGEPGAPGENGTPGQTGARGLPGERGRVGAPGPAGARGSDGSVGPVGPAGPIGSAGPPGFPGAPGPKGEIGAVGNAGPAGPAGPRGEVGLPGLSGPVGPPGNPGANGLTGAKGAAGLPGVAGAPGLPGPRGIPGPVGAAGATGARGLVGEPGPAGSKGESGNKGEPGSAGPQGPPGPSGEEGKRGPNGEAGSAGPPGPPGLRGSPGSRGLPGADGRAGVMGPPGSRGASGPAGVRGPNGDAGRPGEPGLMGPRGLPGSPGNIGPAGKEGPVGLPGIDGRPGPIGPAGARGEPGNIGFPGPKGPTGDPGKNGDKGHAGLAGARGAPGPDGNNGAQGPPGPQGVQGGKGEQGPPGPPGFQGLPGPSGPAGEVGKPGERGLHGEFGLPGPAGPRGERGPPGESGAAGPTGPIGSRGPSGPPGPDGNKGEPGVVGAVGTAGPSGPSGLPGERGAAGIPGGKGEKGEPGLRGEIGNPGRDGARGAPGAVGAPGPAGATGDRGEAGAAGPAGPAGPRGSPGERGEVGPAGPNGFAGPAGAAGQPGAKGERGAKGPKGENGVVGPTGPVGAAGPAGPNGPPGPAGSRGDGGPPGMTGFPGAAGRTGPPGPSGISGPPGPPGPAGKEGLRGPRGDQGPVGRTGEVGAVGPPGFAGEKGPSGEAGTAGPPGTPGPQGLLGAPGILGLPGSRGERGLPGVAGAVGEPGPLGIAGPPGARGPPGAVGSPGVNGAPGEAGRDGNPGNDGPPGRDGQPGHKGERGYPGNIGPVGAAGAPGPHGPVGPAGKHGNRGETGPSGPVGPAGAVGPRGPSGPQGIRGDKGEPGEKGPRGLPGLKGHNGLQGLPGIAGHHGDQGAPGSVGPAGPRGPAGPSGPAGKDGRTGHPGTVGPAGIRGPQGHQGPAGPPGPPGPPGPPGVSGGGYDFGYDGDFYRADQPRSAPSLRPKDYEVDATLKSLNNQIETLLTPEGSRKNPARTCRDLRLSHPEWSSGYYWIDPNQGCTMDAIKVYCDFSTGETCIRAQPENIPAKNWYRSSKDKKHVWLGETINAGSQFEYNVEGVTSKEMATQLAFMRLLANYASQNITYHCKNSIAYMDEETGNLKKAVILQGSNDVELVAEGNSRFTYTVLVDGCSKKTNEWGKTIIEYKTNKPSRLPFLDIAPLDIGGADQEFFVDIGPVCFK

SEQ ID NO: 13 - protein, 2386 aa, Homo sapiens
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDTTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTNIDRPKGLAFTDVDVDSIKIAWESPQGQVSRYRVTYSSPEDGIHELFPAPDGEEDTAELQGLRPGSEYTVSVVALHDDMESQPLIGTQSTAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTVQKTPFVTHPGYDTGNGIQLPGTSGQQPSVGQQMIFEEHGFRRTTPPTTATPIRHRPRPYPPNVGEEIQIGHIPREDVDYHLYPHGPGLNPNASTGQEALSQTTISWAPFQDTSEYIISCHPVGTDEEPLQFRVPGTSTSATLTGLTRGATYNVIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 14 - protein, 2871 aa, Homo sapiens
MRRGRLLEIALGFTVLLASYTSHGADANLEAGNVKETRASRAKRRGGGGHDALKGPNVCGSRYNAYCCPGWKTLPGGNQCIVPICRHSCGDGFCSRPNMCTCPSGQIAPSCGSRSIQHCNIRCMNGGSCSDDHCLCQKGYIGTHCGQPVCESGCLNGGRCVAPNRCACTYGFTGPQCERDYRTGPCFTVISNQMCQGQLSGIVCTKTLCCATVGRAWGHPCEMCPAQPHPCRRGFIPNIRTGACQDVDECQAIPGLCQGGNCINTVGSFECKCPAGHKLNEVSQKCEDIDECSTIPGICEGGECTNTVSSYFCKCPPGFYTSPDGTRCIDVRPGYCYTALTNGRCSNQLPQSITKMQCCCDAGRCWSPGVTVAPEMCPIRATEDFNKLCSVPMVIPGRPEYPPPPLGPIPPVLPVPPGFPPGPQIPVPRPPVEYLYPSREPPRVLPVNVTDYCQLVRYLCQNGRCIPTPGSYRCECNKGFQLDLRGECIDVDECEKNPCAGGECINNQGSYTCQCRAGYQSTLTRTECRDIDECLQNGRICNNGRCINTDGSFHCVCNAGFHVTRDGKNCEDMDECSIRNMCLNGMCINEDGSFKCICKPGFQLASDGRYCKDINECETPGICMNGRCVNTDGSYRCECFPGLAVGLDGRVCVDTHMRSTCYGGYKRGQCIKPLFGAVTKSECCCASTEYAFGEPCQPCPAQNSAEYQALCSSGPGMTSAGSDINECALDPDICPNGICENLRGTYKCICNSGYEVDSTGKNCVDINECVLNSLLCDNGQCRNTPGSFVCTCPKGFIYKPDLKTCEDIDECESSPCINGVCKNSPGSFICECSSESTLDPTKTICIETIKGTCWQTVIDGRCEININGATLKSQCCSSLGAAWGSPCTLCQVDPICGKGYSRIKGTQCEDIDECEVFPGVCKNGLCVNTRGSFKCQCPSGMTLDATGRICLDIRLETCFLRYEDEECTLPIAGRHRMDACCCSVGAAWGTEECEECPMRNTPEYEELCPRGPGFATKEITNGKPFFKDINECKMIPSLCTHGKCRNTIGSFKCRCDSGFALDSEERNCTDIDECRISPDLCGRGQCVNTPGDFECKCDEGYESGFMMMKNCMDIDECQRDPLLCRGGVCHNTEGSYRCECPPGHQLSPNISACIDINECELSAHLCPNGRCVNLIGKYQCACNPGYHSTPDRLFCVDIDECSIMNGGCETFCTNSEGSYECSCQPGFALMPDQRSCTDIDECEDNPNICDGGQCTNIPGEYRCLCYDGFMASEDMKTCVDVNECDLNPNICLSGTCENTKGSFICHCDMGYSGKKGKTGCTDINECEIGAHNCGKHAVCTNTAGSFKCSCSPGWIGDGIKCTDLDECSNGTHMCSQHADCKNTMGSYRCLCKEGYTGDGFTCTDLDECSENLNLCGNGQCLNAPGGYRCECDMGFVPSADGKACEDIDECSLPNICVFGTCHNLPGLFRCECEIGYELDRSGGNCTDVNECLDPTTCISGNCVNTPGSYICDCPPDFELNPTRVGCVDTRSGNCYLDIRPRGDNGDTACSNEIGVGVSKASCCCSLGKAWGTPCEMCPAVNTSEYKILCPGGEGFRPNPITVILEDIDECQELPGLCQGGKCINTFGSFQCRCPTGYYLNEDTRVCDDVNECETPGICGPGTCYNTVGNYTCICPPDYMQVNGGNNCMDMRRSLCYRNYYADNQTCDGELLFNMTKKMCCCSYNIGRAWNKPCEQCPIPSTDEFATLCGSQRPGFVIDIYTGLPVDIDECREIPGVCENGVCINMVGSFRCECPVGFFYNDKLLVCEDIDECQNGPVCQRNAECINTAGSYRCDCKPGYRFTSTGQCNDRNECQEIPNICSHGQCIDTVGSFYCLCHTGFKTNDDQTMCLDINECERDACGNGTCRNTIGSFNCRCNHGFILSHNNDCIDVDECASGNGNLCRNGQCINTVGSFQCQCNEGYEVAPDGRTCVDINECLLEPRKCAPGTCQNLDGSYRCICPPGYSLQNEKCEDIDECVEEPEICALGTCSNTEGSFKCLCPEGFSLSSSGRRCQDLRMSYCYAKFEGGKCSSPKSRNHSKQECCCALKGEGWGDPCELCPTEPDEAFRQICPYGSGIIVGPDDSAVDMDECKEPDVCKHGQCINTDGSYRCECPFGYILAGNECVDTDECSVGNPCGNGTCKNVIGGFECTCEEGFEPGPMMTCEDINECAQNPLLCAFRCVNTYGSYECKCPVGYVLREDRRMCKDEDECEEGKHDCTEKQMECKNLIGTYMCICGPGYQRRPDGEGCVDENECQTKPGICENGRCLNTRGSYTCECNDGFTASPNQDECLDNREGYCFTEVLQNMCQIGSSNRNPVTKSECCCDGGRGWGPHCEICPFQGTVAFKKLCPHGRGFMTNGADIDECKVIHDVCRNGECVNDRGSYHCICKTGYTPDITGTSCVDLNECNQAPKPCNFICKNTEGSYQCSCPKGYILQEDGRSCKDLDECATKQHNCQFLCVNTIGGFTCKCPPGFTQHHTSCIDNNECTSDINLCGSKGICQNTPGSFTCECQRGFSLDQTGSSCEDVDECEGNHRCQHGCQNIIGGYRCSCPQGYLQHYQWNQCVDENECLSAHICGGASCHNTLGSYKCMCPAGFQYEQFSGGCQDINECGSAQAPCSYGCSNTEGGYLCGCPPGYFRIGQGHCVSGMGMGRGNPEPPVSGEMDDNSLSPEACYECKINGYPKRGRKRRSTNETDASNIEDQSETEANVSLASWDVEKTAIFAFNISHVSNKVRILELLPALTTLTNHNRYLIESGNEDGFFKINQKEGISYLHFTKKKPVAGTYSLQISSTPLYKKKELNQLEDKYDKDYLSGELGDNLKMKIQVLLH

SEQ ID NO: 15 - protein, 201 aa, Homo sapiens [sequence omitted]

SEQ ID NO: 16 - protein, 207 aa, Homo sapiens
MAPFEPLASGILLLLWLIAPSRACTCVPPHPQTAFCNSDLVIRAKFVGTPEVNQTTLYQRYEIKMTKMYKGFQALGDAADIRFVYTPAMESVCGYFHRSHNRSEEFLIAGKLQDGLLHITTCSFVAPWNSLSLAQRRGFTKTYTVGCEECTVFPCLSIPCKLQSGTHCLWTDQLLQGSEKGFQSRHLACLPREPGLCTWQSLRSQIA

SEQ ID NO: 17 - protein, 135 aa, Homo sapiens
MACGLVASNLNLKPGECLRVRGEVAPDAKSFVLNLGKDSNNLCLHFNPRFNAHGDANTIVCNSKDGGAWGTEQREAVFPFQPGSVAEVCITFDQANLTVKLPDGYEFKFPNRLNLEAINYMAADGDFKIKCVAFD

SEQ ID NO: 18 - protein, 338 aa, Homo sapiens [sequence omitted]

SEQ ID NO: 19 - protein, 382 aa, Homo sapiens
MRSPLCWLLPLLILASVAQGQPTRRPRPGTGPGRRPRPRPRPTPSFPQPDEPAEPTDLPPPLPPGPPSIFPDCPRECYCPPDFPSALYCDSRNLRKVPVIPPRIHYLYLQNNFITELPVESFQNATGLRWINLDNNRIRKIDQRVLEKLPGLVFLYMEKNQLEEVPSALPRNLEQLRLSQNHISRIPPGVFSKLENLLLLDLQHNRLSDGVFKPDTFHGLKNLMQLNLAHNILRKMPPRVPTAIHQLYLDSNKIETIPNGYFKSFPNLAFIRLNYNKLTDRGLPKNSFNISNLLVLHLSHNRISSVPAINNRLEHLYLNNNSIEKINGTQICPNDLVAFHDFSSDLENVPHLRYLRLDGNYLKPPIPLDLMMCFRLLQSVVI

SEQ ID NO: 20 - protein, 2201 aa, Homo sapiens
MGAMTQLLAGVFLAFLALATEGGVLKKVIRHKRQSGVNATLPEENQPVVFNHVYNIKLPVGSQCSVDLESASGEKDLAPPSEPSESFQEHTVDGENQIVFTHRINIPRRACGCAAAPDVKELLSRLEELENLVSSLREQCTAGAGCCLQPATGRLDTRPFCSGRGNFSTEGCGCVCEPGWKGPNCSEPECPGNCHLRGRCIDGQCICDDGFTGEDCSQLACPSDCNDQGKCVNGVCICFEGYAGADCSREICPVPCSEEHGTCVDGLCVCHDGFAGDDCNKPLCLNNCYNRGRCVENECVCDEGFTGEDCSELICPNDCFDRGRCINGTCYCEEGFTGEDCGKPTCPHACHTQGRCEEGQCVCDEGFAGVDCSEKRCPADCHNRGRCVDGRCECDDGFTGADCGELKCPNGCSGHGRCVNGQCVCDEGYTGEDCSQLRCPNDCHSRGRCVEGKCVCEQGFKGYDCSDMSCPNDCHQHGRCVNGMCVCDDGYTGEDCRDRQCPRDCSNRGLCVDGQCVCEDGFTGPDCAELSCPNDCHGQGRCVNGQCVCHEGFMGKDCKEQRCPSDCHGQGRCVDGQCICHEGFTGLDCGQHSCPSDCNNLGQCVSGRCICNEGYSGEDCSEVSPPKDLVVTEVTEETVNLAWDNEMRVTEYLVVYTPTHEGGLEMQFRVPGDQTSTIIQELEPGVEYFIRVFAILENKKSIPVSARVATYLPAPEGLKFKSIKETSVEVEWDPLDIAFETWEIIFRNMNKEDEGEITKSLRRPETSYRQTGLAPGQEYEISLHIVKNNTRGPGLKRVTTTRLDAPSQIEVKDVTDTTALITWFKPLAEIDGIELTYGIKDVPGDRTTIDLTEDENQYSIGNLKPDTEYEVSLISRRGDMSSNPAKETFTTGLDAPRNLRRVSQTDNSITLEWRNGKAAIDSYRIKYAPISGGDHAEVDVPKSQQATTKTTLTGLRPGTEYGIGVSAVKEDKESNPATINAATELDTPKDLQVSETAETSLTLLWKTPLAKFDRYRLNYSLPTGQWVGVQLPRNTTSYVLRGLEPGQEYNVLLTAEKGRHKSKPARVKASTEQAPELENLTVTEVGWDGLRLNWTAADQAYEHFIIQVQEANKVEAARNLTVPGSLRAVDIPGLKAATPYTVSIYGVIQGYRTPVLSAEASTGETPNLGEVVVAEVGWDALKLNWTAPEGAYEYFFIQVQEADTVEAAQNLTVPGGLRSTDLPGLKAATHYTITIRGVTQDFSTTPLSVEVLTEEVPDMGNLTVTEVSWDALRLNWTTPDGTYDQFTIQVQEADQVEEAHNLTVPGSLRSMEIPGLRAGTPYTVTLHGEVRGHSTRPLAVEVVTEDLPQLGDLAVSEVGWDGLRLNWTAADNAYEHFVIQVQEVNKVEAAQNLTLPGSLRAVDIPGLEAATPYRVSIYGVIRGYRTPVLSAEASTAKEPEIGNLNVSDITPESFNLSWMATDGIFETFTIEIIDSNRLLETVEYNISGAERTAHISGLPPSTDFIVYLSGLAPSIRTKTISATATTEALPLLENLTISDINPYGFTVSWMASENAFDSFLVTVVDSGKLLDPQEFTLSGTQRKLELRGLITGIGYEVMVSGFTQGHQTKPLRAEIVTEAEPEVDNLLVSDATPDGFRLSWTADEGVFDNFVLKIRDTKKQSEPLEITLLAPERTRDITGLREATEYEIELYGISKGRRSQTVSAIATTAMGSPKEVIFSDITENSATVSWRAPTAQVESFRITYVPITGGTPSMVTVDGTKTQTRLVKLIPGVEYLVSIIAMKGFEESEPVSGSFTTALDGPSGLVTANITDSEALARWQPAIATVDSYVISYTGEKVPEITRTVSGNTVEYALTDLEPATEYTLRIFAEKGPQKSSTITAKFTTDLDSPRDLTATEVQSETALLTWRPPRASVTGYLLVYESVDGTVKEVIVGPDTTSYSLADLSPSTHYTAKIQALNGPLRSNMIQTIFTTIGLLYPFPKDCSQAMLNGDTTSGLYTIYLNGDKAEALEVFCDMTSDGGGWIVFLRRKNGRENFYQNWKAYAAGFGDRREEFWLGLDNLNKITAQGQYELRVDLRDHGETAFAVYDKFSVGDAKTRYKLKVEGYSGTAGDSMAYHNGRSFSTFDKDTDSAITNCALSYKGAFWYRNCHRVNLMGRYGDNNHSQGVNWFHWKGHEHSIQFAEMKLRPSNFRNLEGRRKRA

SEQ ID NO: 21 - protein, 478 aa, Homo sapiens
MAPLRPLLILALLAWVALADQESCKGRCTEGFNVDKKCQCDELCSYYQSCCTDYTAECKPQVTRGDVFTMPEDEYTVYDDGEEKNNATVHEQVGGPSLTSDLQAQSKGNPEQTPVLKPEEEAPAPEVGASKPEGIDSRPETLHPGRPQPPAEEELCSGKPFDAFTDLKNGSLFAFRGQYCYELDEKAVRPGYPKLIRDVWGIEGPIDAAFTRINCQGKTYLFKGSQYWRFEDGVLDPDYPRNISDGFDGIPDNVDAALALPAHSYSGRERVYFFKGKQYWEYQFQHQPSQEECEGSSLSAVFEHFAMMQRDSWEDIFELLFWGRTSAGTRQPQFISRDWHGVPGQVDAAMAGRIYISGMAPRPSLAKKQRFRHRNRKGYRSQRGHSRGRNQNSRRPSRATWLSLFSSEESNLGANNYDDYRMDWLVPATCEPIQSVFFFSGDKYYRVNLRTRRVDTVDPPYPRSIAQYWLGCPAPGHL

SEQ ID NO: 22 - protein, 4289 aa, Homo sapiens
MMPAQYALTSSLVLLVLLSTARAGPFSSRSNVTLPAPRPPPQPGGHTVGAGVGSPSSQLYEHTVEGGEKQVVFTHRINLPPSTGCGCPPGTEPPVLASEVQALRVRLEILEELVKGLKEQCTGGCCPASAQAGTGQTDVRTLCSLHGVFDLSRCTCSCEPGWGGPTCSDPTDAEIPPSSPPSASGSCPDDCNDQGRCVRGRCVCFPGYTGPSCGWPSCPGDCQGRGRCVQGVCVCRAGFSGPDCSQRSCPRGCSQRGRCEGGRCVCDPGYTGDDCGMRSCPRGCSQRGRCENGRCVCNPGYTGEDCGVRSCPRGCSQRGRCKDGRCVCDPGYTGEDCGTRSCPWDCGEGGRCVDGRCVCWPGYTGEDCSTRTCPRDCRGRGRCEDGECICDTGYSGDDCGVRSCPGDCNQRGRCEDGRCVCWPGYTGTDCGSRACPRDCRGRGRCENGVCVCNAGYSGEDCGVRSCPGDCRGRGRCESGRCMCWPGYTGRDCGTRACPGDCRGRGRCVDGRCVCNPGFTGEDCGSRRCPGDCRGHGLCEDGVCVCDAGYSGEDCSTRSCPGGCRGRGQCLDGRCVCEDGYSGEDCGVRQCPNDCSQHGVCQDGVCICWEGYVSEDCSIRTCPSNCHGRGRCEEGRCLCDPGYTGPTCATRMCPADCRGRGRCVQGVCLCHVGYGGEDCGQEEPPASACPGGCGPRELCRAGQCVCVEGFRGPDCAIQTCPGDCRGRGECHDGSCVCKDGYAGEDCGEARVPSSASAYDQRGLAPGQEYQVTVRALRGTSWGLPASKTITTMIDGPQDLRVVAVTPTTLELGWLRPQAEVDRFVVSYVSAGNQRVRLEVPPEADGTLLTDLMPGVEYVVTVTAERGRAVSYPASVRANTEEREEESPPRPSLSQPPRRPWGNLTAELSRFRGTVQDLERHLRAHGYPLRANQTYTSVARHIHEYLQRQVLGSSADGALLVSLDGLRGQFERVVLRWRPQPPAEGPGGELTVPGTTRTVSLPDLRPGTTYHVEVHGVRAGQTSKSYAFITTTGPSTTQGAQAPLLQQRPQELGELRVLGRDETGRLRVVWTAQPDTFAYFQLRMRVPEGPGAHEEVLPGDVRQALVPPPPPGTPYELSLHGVPPGGKPSDPIIYQGIMDKDEEKPGKSSGPPRLGELTVTDRTSDSLLLRWTVPEGEFDSFVIQYKDRDGQPQVVPVEGPQRSAVITSLDPGRKYKFVLYGFVGKKRHGPLVAEAKILPQSDPSPGTPPHLGNLWVTDPTPDSLHLSWTVPEGQFDTFMVQYRDRDGRPQVVPVEGPERSFVVSSLDPDHKYRFTLFGIANKKRYGPLTADGTTAPERKEEPPRPEFLEQPLLGELTVTGVTPDSLRLSWTVAQGPFDSFMVQYKDAQGQPQAVPVAGDENEVTVPGLDPDRKYKMNLYGLRGRQRVGPESVVAKTAPQEDVDETPSPTELGTEAPESPEEPLLGELTVTGSSPDSLSLFWTVPQGSFDSFTVQYKDRDGRPRAVRVGGKESEVTVGGLEPGHKYKMHLYGLHEGQRVGPVSAVGVTAPQQEETPPATESPLEPRLGELTVTDVTPNSVGLSWTVPEGQFDSFIVQYKDKDGQPQVVPVAADQREVTVYNLEPERKYKMNMYGLHDGQRMGPLSVVIVTAPATEASKPPLEPRLGELTVTDITPDSVGLSWTVPEGEFDSFVVQYKDRDGQPQVVPVAADQREVTIPDLEPSRKYKFLLFGIQDGKRRSPVSVEAKTVARGDASPGAPPRLGELWVTDPTPDSLRLSWTVPEGQFDSFVVQFKDKDGPQVVPVEGHERSVTVTPLDAGRKYRFLLYGLLGKKRHGPLTADGTTEARSAMDDTGTKRPPKPRLGEELQVTTVTQNSVGLSWTVPEGQFDSFVVQYKDRDGQPQVVPVEGSLREVSVPGLDPAHRYKLLLYGLHHGKRVGPISAVAITAGREETETETTAPTPPAPEPHLGELTVEEATSHTLHLSWMVTEGEFDSFEIQYTDRDGQLQMVRIGGDRNDITLSGLESDHRYLVTLYGFSDGKHVGPVHVEALTVPEEEKPSEPPTATPEPPIKPRLGELTVTDATPDSLSLSWTVPEGQFDHFLVQYRNGDGQPKAVRVPGHEEGVTISGLEPDHKYKMNLYGFHGGQRMGPVSVVGVTEPSMEAPEPAEEPLLGELTVTGSSPDSLSLSWTVPQGRFDSFTVQYKDRDGRPQVVRVGGEESEVTVGGLEPGRKYKMHLYGLHEGRRVGPVSAVGVTAPEEESPDAPLAKLRLGQMTVRDITSDSLSLSWTVPEGQFDHFLVQFKNGDGQPKAVRVPGHEDGVTISGLEPDHKYKMNLYGFHGGQRVGPVSAVGLTASTEPPTPEPPIKPRLEELTVTDATPDSLSLSWTVPEGQFDHFLVQYKNGDGQPKATRVPGHEDRVTISGLEPDNKYKMNLYGFHGGQRVGPVSAIGVTEEETPSPTEPSMEAPEPPEEPLLGELTVTGSSPDSLSLSWTVPQGRFDSFTVQYKDRDGRPQVVRVGGEESEVTVGGLEPGRKYKMHLYGLHEGRRVGPVSTVGVTAPQEDVDETPSPTEPGTEAPGPPEEPLLGELTVTGSSPDSLSLSWTVPQGRFDSFTVQYKDRDGRPQAVRVGGQESKVTVRGLEPGRKYKMHLYGLHEGRRLGPVSAVGVTEDEAETTQAVPTMTPEPPIKPRLGELTMTDATPDSLSLSWTVPEGQFDHFLVQYRNGDGQPKAVRVPGHEDGVTISGLEPDHKYKMNLYGFHGGQRVGPISVIGVTEEETPSPTELSTEAPEPPEEPLLGELTVTGSSPDSLSLSWTIPQGHFDSFTVQYKDRDGRPQVMRVRGEESEVTVGGLEPGRKYKMHLYGLHEGRRVGPVSTVGVTVPTTTPEPPNKPRLGELTVTDATPDSLSLSWMVPEGQFDHFLVQYRNGDGQPKVVRVPGHEDGVTISGLEPDHKYKMNLYGFHGGQRVGPISVIGVTEEETPAPTEPSTEAPEPPEEPLLGELTVTGSSPDSLSLSWTIPQGRFDSFTVQYKDRDGRPQVVRVRGEESEVTVGGLEPGCKYKMHLYGLHEGQRVGPVSAVGVTAPKDEAETTQAVPTMTPEPPIKPRLGELTVTDATPDSLSLSWMVPEGQFDHFLVQYRNGDGQPKAVRVPGHEDGVTISGLEPDHKYKMNLYGFHGGQRVGPVSAIGVTEEETPSPTEPSTEAPEAPEEPLLGELTVTGSSPDSLSLSWTVPQGRFDSFTVQYKDRDGQPQVVRVRGEESEVTVGGLEPGRKYKMHLYGLHEGQRVGPVSTVGITAPLPTPLPVEPRLGELAVAAVTSDSVGLSWTVAQGPFDSFLVQYRDAQGQPQAVPVSGDLRAVAVSGLDPARKYKFLLFGLQNGKRHGPVPVEARTAPDTKPSPRLGELTVTDATPDSVGLSWTVPEGEFDSFVVQYKDKDGRLQVVPVAANQREVTVQGLEPSRKYRFLLYGLSGRKRLGPISADSTTAPLEKELPPHLGELTVAEETSSSLRLSWTVAQGPFDSFVVQYRDTDGQPRAVPVAADQRTVTVEDLEPGKKYKFLLYGLLGGKRLGPVSALGMTAPEEDTPAPELAPEAPEPPEEPRLGVLTVTDTTPDSMRLSWSVAQGPFDSFVVQYEDTNGQPQALLVDGDQSKILISGLEPSTPYRFLLYGLHEGKRLGPLSAEGTTGLAPAGQTSEESRPRLSQLSVTDVTTSSLRLNWEAPPGAFDSFLLRFGVPSPSTLEPHPRPLLQRELMVPGTRHSAVLRDLRSGTLYSLTLYGLRGPHKADSIQGTARTLSPVLESPRDLQFSEIRETSAKVNWMPPPSRADSFKVSYQLADGGEPQSVQVDGQARTQKLQGLIPGARYEVTVVSVRGFEESEPLTGFLTTVPDGPTQLRALNLTEGFAVLHWKPPQNPVDTYDVQVTAPGAPPLQAETPGSAVDYPLHDLVLHTNYTATVRGLRGPNLTSPASITFTTGLEAPRDLEAKEVTPRTALLTWTEPPVRPAGYLLSFHTPGGQNQEILLPGGITSHQLLGLFPSTSYNARLQAMWGQSLLPPVSTSFTTGGLRIPFPRDCGEEMQNGAGASRTSTIFLNGNRERPLNVFCDMETDGGGWLVFQRRMDGQTDFWRDWEDYAHGFGNISGEFWLGNEALHSLTQAGDYSMRVDLRAGDEAVFAQYDSFHVDSAAEYYRLHLEGYHGTAGDSMSYHSGSVFSARDRDPNSLLISCAVSYRGAWWYRNCHYANLNGLYGSTVDHQGVSWYHWKGFEFSVPFTEMKLRPRNFRSPAGGG

SEQ ID NO: 23 - protein, 1712 aa, Homo sapiens
MGRDQRAVAGPALRRWLLLGTVTVGFLAQSVLAGVKKFDVPCGGRDCSGGCQCYPEKGGRGQPGPVGPQGYNGPPGLQGFPGLQGRKGDKGERGAPGVTGPKGDVGARGVSGFPGADGIPGHPGQGGPRGRPGYDGCNGTQGDSGPQGPPGSEGFTGPPGPQGPKGQKGEPYALPKEERDRYRGEPGEPGLVGFQGPPGRPGHVGQMGPVGAPGRPGPPGPPGPKGQQGNRGLGFYGVKGEKGDVGQPGPNGIPSDTLHPIIAPTGVTFHPDQYKGEKGSEGEPGIRGISLKGEEGIMGFPGLRGYPGLSGEKGSPGQKGSRGLDGYQGPDGPRGPKGEAGDPGPPGLPAYSPHPSLAKGARGDPGFPGAQGEPGSQGEPGDPGLPGPPGLSIGDGDQRRGLPGEMGPKGFIGDPGIPALYGGPPGPDGKRGPPGPPGLPGPPGPDGFLFGLKGAKGRAGFPGLPGSPGARGPKGWKGDAGECRCTEGDEAIKGLPGLPGPKGFAGINGEPGRKGDRGDPGQHGLPGFPGLKGVPGNIGAPGPKGAKGDSRTITTKGERGQPGVPGVPGMKGDDGSPGRDGLDGFPGLPGPPGDGIKGPPGDPGYPGIPGTKGTPGEMGPPGLGLPGLKGQRGFPGDAGLPGPPGFLGPPGPAGTPGQIDCDTDVKRAVGGDRQEAIQPGCIGGPKGLPGLPGPPGPTGAKGLRGIPGFAGADGGPGPRGLPGDAGREGFPGPPGFIGPRGSKGAVGLPGPDGSPGPIGLPGPDGPPGERGLPGEVLGAQPGPRGDAGVPGQPGLKGLPGDRGPPGFRGSQGMPGMPGLKGQPGLPGPSGQPGLYGPPGLHGFPGAPGQEGPLGLPGIPGREGLPGDRGDPGDTGAPGPVGMKGLSGDRGDAGFTGEQGHPGSPGFKGIDGMPGTPGLKGDRGSPGMDGFQGMPGLKGRPGFPGSKGEAGFFGIPGLKGLAGEPGFKGSRGDPGPPGPPPVILPGMKDIKGEKGDEGPMGLKGYLGAKGIQGMPGIPGLSGIPGLPGRPGHIKGVKGDIGVPGIPGLPGFPGVAGPPGITGFPGFIGSRGDKGAPGRAGLYGEIGATGDFGDIGDTINLPGRPGLKGERGTTGIPGLKGFFGEKGTEGDIGFPGITGVTGVQGPPGLKGQTGFPGLTGPPGSQGELGRIGLPGGKGDDGWPGAPGLPGFPGLRGIRGLHGLPGTKGFPGSPGSDIHGDPGFPGPPGERGDPGEANTLPGPVGVPGQKGDQGAPGERGPPGSPGLQGFPGITPPSNISGAPGDKGAPGIFGLKGYRGPPGPPGSAALPGSKGDTGNPGAPGTPGTKGWAGDSGPQGRPGVFGLPGEKGPRGEQGFMGNTGPTGAVGDRGPKGPKGDPGFPGAPGTVGAPGIAGIPQKIAVQPGTVGPQGRRGPPGAPGEMGPQGPPGEPGFRGAPGKAGPQGRGGVSAVPGFRGDEGPIGHQGPIGQEGAPGRPGSPGLPGMPGRSVSIGYLLVKHSQTDQEPMCPVGMNKLWSGYSLLYFEGQEKAHNQDLGLAGSCLARFSTMPFLYCNPGDVCYYASRNDKSYWLSTTAPLPMMPVAEDEIKPYISRCSVCEAPAIAIAVHSQDVSIPHCPAGWRSLWIGYSFLMHTAAGDEGGGQSLVSPGSCLEDFRATPFIECNGGRGTCHYYANKYSFWLTTIPEQSFQGSPSADTLKAGLIRTHISRCQVCMKNL

SEQ ID NO: 24 - protein, 1691 aa, Homo sapiens
MKLRGVSLAAGLFLLALSLWGQPAEAAACYGCSPGSKCDCSGIKGEKGERGFPGLEGHPGLPGFPGPEGPPGPRGQKGDDGIPGPPGPKGIRGPPGLPGFPGTPGLPGMPGHDGAPGPQGIPGCNGTKGERGFPGSPGFPGLQGPPGPPGIPGMKGEPGSIIMSSLPGPKGNPGYPGPPGIQGLPGPTGIPGPIGPPGPPGLMGPPGPPGLPGPKGNMGLNFQGPKGEKGEQGLQGPPGPPGQISEQKRPIDVEFQKGDQGLPGDRGPPGPPGIRGPPGPPGGEKGEKGEQGEPGKRGKPGKDGENGQPGIPGLPGDPGYPGEPGRDGEKGQKGDTGPPGPPGLVIPRPGTGITIGEKGNIGLPGLPGEKGERGFPGIQGPPGLPGPPGAAVMGPPGPPGFPGERGQKGDEGPPGISIPGPPGLDGQPGAPGLPGPPGPAGPHIPPSDEICEPGPPGPPGSPGDKGLQGEQGVKGDKGDTCFNCIGTGISGPPGQPGLPGLPGPPGSLGFPGQKGEKGQAGATGPKGLPGIPGAPGAPGFPGSKGEPGDILTFPGMKGDKGELGSPGAPGLPGLPGTPGQDGLPGLPGPKGEPGGITFKGERGPPGNPGLPGLPGNIGPMGPPGFGPPGPVGEKGIQGVAGNPGQPGIPGPKGDPGQTITQPGKPGLPGNPGRDGDVGLPGDPGLPGQPGLPGIPGSKGEPGIPGIGLPGPPGPKGFPGIPGPPGAPGTPGRIGLEGPPGPPGFPGPKGEPGFALPGPPGPPGLPGFKGALGPKGDRGFPGPPGPPGRTGLDGLPGPKGDVGPNGQPGPMGPPGLPGIGVQGPPGPPGIPGPIGQPGLHGIPGEKGDPGPPGLDVPGPPGERGSPGIPGAPGPIGPPGSPGLPGKAGASGFPGTKGEMGMMGPPGPPGPLGIPGRSGVPGLKGDDGLQGQPGLPGPTGEKGSKGEPGLPGPPGPMDPNLLGSKGEKGEPGLPGIPGVSGPKGYQGLPGDPGQPGLSGQPGLPGPPGPKGNPGLPGQPGLIGPPGLKGTIGDMGFPGPQGVEGPPGPSGVPGQPGSPGLPGQKGDKGDPGISSIGLPGLPGPKGEPGLPGYPGNPGIKGSVGDPGLPGLPGTPGAKGQPGLPGFPGTPGPPGPKGISGPPGNPGLPGEPGPVGGGGHPGQPGPPGEKGKPGQDGIPGPAGQKGEPGQPGFGNPGPPGLPGLSGQKGDGGLPGIPGNPGLPGPKGEPGFHGFPGVQGPPGPPGSPGPALEGPKGNPGPQGPPGRPGPTGFQGLPGPEGPPGLPGNGGIKGEKGNPGQPGLPGLPGLKGDQGPPGLQGNPGRPGLNGMKGDPGLPGVPGFPGMKGPSGVPGSAGPEGEPGLIGPPGPPGLPGPSGQSIIIKGDAGPPGIPGQPGLKGLPGPQGPQGLPGPTGPPGDPGRNGLPGFDGAGGRKGDPGLPGQPGTRGLDGPPGPDGLQGPPGPPGTSSVAHGFLITRHSQTTDAPQCPQGTLQVYEGFSLLYVQGNKRAHGQDLGTAGSCLRRFSTMPFMFCNINNVCNFASRNDYSYWLSTPEPMPMSMQPLKGQSIQPFISRCAVCEAPAVVIAVHSQTIQIPHCPQGWDSLWIGYSFMMHTSAGAEGSGQALASPGSCLEEFRSAPFIECHGRGTCNYYANSYSFWLATVDVSDMFSKPQSETLKAGDLRTRISRCQVCMKRT

SEQ ID NO: 25 - protein, 730 aa, Homo sapiens
MAGLTAAAPRPGVLLLLLSILHPSRPGGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPSVGGVPGVGGVPGVGISPEAQAAAAAKAAKYGVGTPAAAAAKAAAKAAQFALLNLAGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 26 - protein, 16 aa, Artificial Sequence
  Synthetic amino acid sequence
TGGISVPGPMGPSGPR

SEQ ID NO: 27 - protein, 13 aa, Artificial Sequence
  Synthetic amino acid sequence
GLPGPPGAPGPQG

SEQ ID NO: 28 - protein, 14 aa, Artificial Sequence
  Synthetic amino acid sequence
GLPGPPGAPGPQGF

SEQ ID NO: 29 - protein, 24 aa, Artificial Sequence
  Synthetic amino acid sequence
PGEPGEPGASGPMGPRGPPGPPGK

SEQ ID NO: 30 - protein, 17 aa, Artificial Sequence
  Synthetic amino acid sequence
GASGPMGPRGPPGPPGK

SEQ ID NO: 31 - protein, 17 aa, Artificial Sequence
  Synthetic amino acid sequence
KPGRPGERGPPGPQGAR

SEQ ID NO: 32 - protein, 20 aa, Artificial Sequence
  Synthetic amino acid sequence
GPPGPQGARGLPGTAGLPGM

SEQ ID NO: 33 - protein, 7 aa, Artificial Sequence
  Synthetic amino acid sequence
AGPQGPR

SEQ ID NO: 34 - protein, 15 aa, Artificial Sequence
  Synthetic amino acid sequence
GAPGIAGAPGFPGAR

SEQ ID NO: 35 - protein, 28 aa, Artificial Sequence
  Synthetic amino acid sequence
PGIAGAPGFPGARGPSGPQGPGGPPGPK

SEQ ID NO: 36 - protein, 26 aa, Artificial Sequence
  Synthetic amino acid sequence
IAGAPGFPGARGPSGPQGPGGPPGPK

SEQ ID NO: 37 - protein, 21 aa, Artificial Sequence
  Synthetic amino acid sequence
GFPGARGPSGPQGPGGPPGPK

SEQ ID NO: 38 - protein, 10 aa, Artificial Sequence
  Synthetic amino acid sequence
GPSGPQGPGG

SEQ ID NO: 39 - protein, 9 aa, Artificial Sequence
  Synthetic amino acid sequence
GDTGAKGEP

SEQ ID NO: 40 - protein, 13 aa, Artificial Sequence
  Synthetic amino acid sequence
VQGPPGPAGEEGK

SEQ ID NO: 41 - protein, 13 aa, Artificial Sequence
  Synthetic amino acid sequence
GEPGPTGLPGPPG

SEQ ID NO: 42 - protein, 20 aa, Artificial Sequence
  Synthetic amino acid sequence
GEPGPTGLPGPPGERGGPGS

SEQ ID NO: 43 - protein, 10 aa, Artificial Sequence
  Synthetic amino acid sequence
TGLPGPPGER

SEQ ID NO: 44 - protein, 8 aa, Artificial Sequence
  Synthetic amino acid sequence
LPGPPGER

SEQ ID NO: 45 - protein, 10 aa, Artificial Sequence
  Synthetic amino acid sequence
AGPKGPAGER

SEQ ID NO: 46 - protein, 22 aa, Artificial Sequence
  Synthetic amino acid sequence
GSPGPAGPKGSPGEAGRPGEAG

SEQ ID NO: 47 - protein, 30 aa, Artificial Sequence
  Synthetic amino acid sequence
PGEAGRPGEAGLPGAKGLTGSPGSPGPDGK

SEQ ID NO: 48 - protein, 13 aa, Artificial Sequence
  Synthetic amino acid sequence
LTGSPGSPGPDGK

SEQ ID NO: 49 - protein, 23 aa, Artificial Sequence
  Synthetic amino acid sequence
TGPPGPAGQDGRPGPPGPPGARG

SEQ ID NO: 50 - protein, 28 aa, Artificial Sequence
  Synthetic amino acid sequence
PGAVGPAGKDGEAGAQGPPGPAGPAGER

SEQ ID NO: 51 - protein, 18 aa, Artificial Sequence
  Synthetic amino acid sequence
GEAGAQGPPGPAGPAGER

SEQ ID NO: 52 - protein, 17 aa, Artificial Sequence
  Synthetic amino acid sequence
EAGAQGPPGPAGPAGER

SEQ ID NO: 53 - protein, 11 aa, Artificial Sequence
  Synthetic amino acid sequence
VQGPPGPAGPR

SEQ ID NO: 54 - protein, 10 aa, Artificial Sequence
  Synthetic amino acid sequence
QGPPGPAGPR

SEQ ID NO: 55 - protein, 9 aa, Artificial Sequence
  Synthetic amino acid sequence
GPPGPAGPR

SEQ ID NO: 56 - protein, 35 aa, Artificial Sequence
  Synthetic amino acid sequence
ANGAPGNDGAKGDAGAPGAPGSQGAPGLQGMPGER

SEQ ID NO: 57 - protein, 8 aa, Artificial Sequence
  Synthetic amino acid sequence
LQGMPGER

SEQ ID NO: 58 - protein, 17 aa, Artificial Sequence
  Synthetic amino acid sequence
LTGPIGPPGPAGAPGDK

SEQ ID NO: 59 - protein, 13 aa, Artificial Sequence
  Synthetic amino acid sequence
IGPPGPAGAPGDK

SEQ ID NO: 60 - protein, 16 aa, Artificial Sequence
  Synthetic amino acid sequence
KGESGPSGPAGPTGAR

SEQ ID NO: 61 - protein, 28 aa, Artificial Sequence
  Synthetic amino acid sequence
PGDRGEPGPPGPAGFAGPPGADGQPGAK

SEQ ID NO: 62 - protein, 11 aa, Artificial Sequence
  Synthetic amino acid sequence
GEPGPPGPAGF

SEQ ID NO: 63 - protein, 14 aa, Artificial Sequence
  Synthetic amino acid sequence
FAGPPGADGQPGAK

SEQ ID NO: 64 - protein, 13 aa, Artificial Sequence
  Synthetic amino acid sequence
AGPPGADGQPGAK

SEQ ID NO: 65 - protein, 22 aa, Artificial Sequence
  Synthetic amino acid sequence
RVGPPGPSGNAGPPGPPGPAGK

SEQ ID NO: 66 - protein, 24 aa, Artificial Sequence
  Synthetic amino acid sequence
VGPPGPSGNAGPPGPPGPAGKEGG

SEQ ID NO: 67 - protein, 38 aa, Artificial Sequence
  Synthetic amino acid sequence
EVGPPGPPGPAGEKGSPGADGPAGAPGTPGPQGIAGQR

SEQ ID NO: 68 - protein, 34 aa, Artificial Sequence
  Synthetic amino acid sequence
PGPPGPAGEKGSPGADGPAGAPGTPGPQGIAGQR

SEQ ID NO: 69 - protein, 19 aa, Artificial Sequence
  Synthetic amino acid sequence
GSPGADGPAGAPGTPGPQG

SEQ ID NO: 70 - protein, 18 aa, Artificial Sequence
  Synthetic amino acid sequence
GPAGAPGTPGPQGIAGQR

SEQ ID NO: 71 - protein, 8 aa, Artificial Sequence
  Synthetic amino acid sequence
VVGLPGQR

SEQ ID NO: 72 - protein, 10 aa, Artificial Sequence
  Synthetic amino acid sequence
LAGPPGESGR

SEQ ID NO: 73 - protein, 29 aa, Artificial Sequence
  Synthetic amino acid sequence
ETGPAGPPGAPGAPGAPGPVGPAGKSGDR

SEQ ID NO: 74 - protein, 19 aa, Artificial Sequence
  Synthetic amino acid sequence
RGETGPAGPAGPVGPVGAR

SEQ ID NO: 75 - protein, 11 aa, Artificial Sequence
  Synthetic amino acid sequence
PAGPVGPVGAR

SEQ ID NO: 76 - protein, 8 aa, Artificial Sequence
  Synthetic amino acid sequence
PVGPVGAR

SEQ ID NO: 77 - protein, 17 aa, Artificial Sequence
  Synthetic amino acid sequence
SPGEQGPSGASGPAGPR

SEQ ID NO: 78 - protein, 16 aa, Artificial Sequence
  Synthetic amino acid sequence
PGEQGPSGASGPAGPR

SEQ ID NO: 79 - protein, 12 aa, Artificial Sequence
  Synthetic amino acid sequence
GPSGASGPAGPR

SEQ ID NO: 80 - protein, 8 aa, Artificial Sequence
  Synthetic amino acid sequence
ASGPAGPR

SEQ ID NO: 81 - protein, 12 aa, Artificial Sequence
  Synthetic amino acid sequence
GPPGSAGAPGKD

SEQ ID NO: 82 - protein, 26 aa, Artificial Sequence
  Synthetic amino acid sequence
PPGSAGAPGKDGLNGLPGPIGPPGPR

SEQ ID NO: 83 - protein, 8 aa, Artificial Sequence
  Synthetic amino acid sequence
LPQPPQEK

SEQ ID NO: 84 - protein, 39 aa, Artificial Sequence
  Synthetic amino acid sequence
GLMGPRGPPGAAGAPGPQGFQGPAGEPGEPGQTGPAGAR

SEQ ID NO: 85 - protein, 20 aa, Artificial Sequence
  Synthetic amino acid sequence
FQGPAGEPGEPGQTGPAGAR

SEQ ID NO: 86 - protein, 19 aa, Artificial Sequence
  Synthetic amino acid sequence
QGPAGEPGEPGQTGPAGAR

SEQ ID NO: 87 - protein, 23 aa, Artificial Sequence
  Synthetic amino acid sequence
EDGHPGKPGRPGERGVVGPQGAR

SEQ ID NO: 88 - protein, 35 aa, Artificial Sequence
  Synthetic amino acid sequence
PAGARGSDGSVGPVGPAGPIGSAGPPGFPGAPGPK

SEQ ID NO: 89 - protein, 28 aa, Artificial Sequence
  Synthetic amino acid sequence
DGSVGPVGPAGPIGSAGPPGFPGAPGPK

SEQ ID NO: 90 - protein, 25 aa, Artificial Sequence
  Synthetic amino acid sequence
PGAPGPKGEIGAVGNAGPAGPAGPR

SEQ ID NO: 91 - protein, 9 aa, Artificial Sequence
  Synthetic amino acid sequence
GPAGPAGPR

SEQ ID NO: 92 - protein, 8 aa, Artificial Sequence
  Synthetic amino acid sequence
PAGPAGPR

SEQ ID NO: 93 - protein, 28 aa, Artificial Sequence
  Synthetic amino acid sequence
RGEVGLPGLSGPVGPPGNPGANGLTGAK

SEQ ID NO: 94 - protein, 9 aa, Artificial Sequence
  Synthetic amino acid sequence
GAPGLPGPR

SEQ ID NO: 95 - protein, 17 aa, Artificial Sequence
  Synthetic amino acid sequence
PNGEAGSAGPPGPPGLR

SEQ ID NO: 96 - protein, 17 aa, Artificial Sequence
  Synthetic amino acid sequence
GPRGLPGSPGNIGPAGK

SEQ ID NO: 97 - protein, 12 aa, Artificial Sequence
  Synthetic amino acid sequence
GRPGPIGPAGAR

SEQ ID NO: 98 - protein, 10 aa, Artificial Sequence
  Synthetic amino acid sequence
GPSGPPGPDG

SEQ ID NO: 99 - protein, 27 aa, Artificial Sequence
  Synthetic amino acid sequence
GPSGPPGPDGNKGEPGVVGAVGTAGPS

SEQ ID NO: 100 - protein, 9 aa, Artificial Sequence
  Synthetic amino acid sequence
GPSGLPGER

SEQ ID NO: 101 - protein, 30 aa, Artificial Sequence
  Synthetic amino acid sequence
GAVGAPGPAGATGDRGEAGAAGPAGPAGPR

SEQ ID NO: 102 - protein, 28 aa, Artificial Sequence
  Synthetic amino acid sequence
VGAPGPAGATGDRGEAGAAGPAGPAGPR

SEQ ID NO: 103 - protein, 25 aa, Artificial Sequence
  Synthetic amino acid sequence
PGPAGATGDRGEAGAAGPAGPAGPR

SEQ ID NO: 104 - protein, 28 aa, Artificial Sequence
  Synthetic amino acid sequence
NGVVGPTGPVGAAGPAGPNGPPGPAGSR

SEQ ID NO: 105 - protein, 9 aa, Artificial Sequence
  Synthetic amino acid sequence
GPPGPAGSR

SEQ ID NO: 106 - protein, 24 aa, Artificial Sequence
  Synthetic amino acid sequence
PGPAGSRGDGGPPGMTGFPGAAGR

SEQ ID NO: 107 - protein, 36 aa, Artificial Sequence
  Synthetic amino acid sequence
GDGGPPGMTGFPGAAGRTGPPGPSGISGPPGPPGPA

SEQ ID NO: 108 - protein, 13 aa, Artificial Sequence
  Synthetic amino acid sequence
ISGPPGPPGPAGK

SEQ ID NO: 109 - protein, 20 aa, Artificial Sequence
  Synthetic amino acid sequence
GPSGEAGTAGPPGTPGPQGL

SEQ ID NO: 110 - protein, 10 aa, Artificial Sequence
  Synthetic amino acid sequence
PGILGLPGSR

SEQ ID NO: 111 - protein, 8 aa, Artificial Sequence
  Synthetic amino acid sequence
IAGPPGAR

SEQ ID NO: 112 - protein, 28 aa, Artificial Sequence
  Synthetic amino acid sequence
PGNIGPVGAAGAPGPHGPVGPAGKHGNR

SEQ ID NO: 113 - protein, 10 aa, Artificial Sequence
  Synthetic amino acid sequence
VGPAGAVGPR

SEQ ID NO: 114 - protein, 13 aa, Artificial Sequence
  Synthetic amino acid sequence
QGAPGSVGPAGPR

SEQ ID NO: 115 - protein, 12 aa, Artificial Sequence
  Synthetic amino acid sequence
GPAGPSGPAGKD

SEQ ID NO: 116 - protein, 9 aa, Artificial Sequence
  Synthetic amino acid sequence
GTVGPAGIR

SEQ ID NO: 117 - protein, 17 aa, Artificial Sequence
  Synthetic amino acid sequence
GPQGPKGDPGPPGIPGR

SEQ ID NO: 118 - protein, 45 aa, Artificial Sequence
  Synthetic amino acid sequence
PGTSGHPGSPGSPGYQGPPGEPGQAGPSGPPGPPGAIGPSGPAGK

SEQ ID NO: 119 - protein, 13 aa, Artificial Sequence
  Synthetic amino acid sequence
GLPGPPGIKGPAG

SEQ ID NO: 120 - protein, 28 aa, Artificial Sequence
  Synthetic amino acid sequence
GEVGPAGSPGSNGAPGQRGEPGPQGHAG

SEQ ID NO: 121 - protein, 37 aa, Artificial Sequence
  Synthetic amino acid sequence
GEPGPQGHAGAQGPPGPPGINGSPGGKGEMGPAGIPG

SEQ ID NO: 122 - protein, 25 aa, Artificial Sequence
  Synthetic amino acid sequence
GEMGPAGIPGAPGLMGARGPPGPAG

SEQ ID NO: 123 - protein, 12 aa, Artificial Sequence
  Synthetic amino acid sequence
GIPGAPGLMGAR

SEQ ID NO: 124 - protein, 24 aa, Artificial Sequence
  Synthetic amino acid sequence
GAPGLMGARGPPGPAGANGAPGLR

SEQ ID NO: 125 - protein, 14 aa, Artificial Sequence
  Synthetic amino acid sequence
PAGERGAPGPAGPR

SEQ ID NO: 126 - protein, 15 aa, Artificial Sequence
  Synthetic amino acid sequence
GAPGPAGPRGAAGEP

SEQ ID NO: 127 - protein, 15 aa, Artificial Sequence
  Synthetic amino acid sequence
GEPGRDGVPGGPGMR

SEQ ID NO: 128 - protein, 25 aa, Artificial Sequence
  Synthetic amino acid sequence
DGKPGPPGSQGESGRPGPPGPSGPR

SEQ ID NO: 129 - protein, 24 aa, Artificial Sequence
  Synthetic amino acid sequence
GKPGPPGSQGESGRPGPPGPSGPR

SEQ ID NO: 130 - protein, 12 aa, Artificial Sequence
  Synthetic amino acid sequence
GRPGPPGPSGPR

SEQ ID NO: 131 - protein, 9 aa, Artificial Sequence
  Synthetic amino acid sequence
GPPGPSGPR

SEQ ID NO: 132 - protein, 25 aa, Artificial Sequence
  Synthetic amino acid sequence
QGPPGKNGETGPQGPPGPTGPGGDK

SEQ ID NO: 133 - protein, 11 aa, Artificial Sequence
  Synthetic amino acid sequence
GDAGAPGERGP

SEQ ID NO: 134 - protein, 8 aa, Artificial Sequence
  Synthetic amino acid sequence
LQGMPGER

SEQ ID NO: 135 - protein, 26 aa, Artificial Sequence
  Synthetic amino acid sequence
GEGGPPGVAGPPGGSGPAGPPGPQGV

SEQ ID NO: 136 - protein, 39 aa, Artificial Sequence
  Synthetic amino acid sequence
GSNGNPGPPGPSGSPGKDGPPGPAGNTGAPGSPGVSGPK

SEQ ID NO: 137 - protein, 15 aa, Artificial Sequence
  Synthetic amino acid sequence
NGNPGPPGPSGSPGK

SEQ ID NO: 138 - protein, 9 aa, Artificial Sequence
  Synthetic amino acid sequence
GSPGAQGPP

SEQ ID NO: 139 - protein, 11 aa, Artificial Sequence
  Synthetic amino acid sequence
GNPGSDGLPGR

SEQ ID NO: 140 - protein, 25 aa, Artificial Sequence
  Synthetic amino acid sequence
ENGSPGAPGAPGHPGPPGPVGPAGK

SEQ ID NO: 141 - protein, 21 aa, Artificial Sequence
  Synthetic amino acid sequence
PGAPGAPGHPGPPGPVGPAGK

SEQ ID NO: 142 - protein, 19 aa, Artificial Sequence
  Synthetic amino acid sequence
RGESGPAGPAGAPGPAGSR

SEQ ID NO: 143 - protein, 12 aa, Artificial Sequence
  Synthetic amino acid sequence
GESGPAGPAGAP

SEQ ID NO: 144 - protein, 25 aa, Artificial Sequence
  Synthetic amino acid sequence
PGAPGSPGPAGQQGAIGSPGPAGPR

SEQ ID NO: 145 - protein, 26 aa, Artificial Sequence
  Synthetic amino acid sequence
GQQGAIGSPGPAGPRGPVGPSGPPGK

SEQ ID NO: 146 - protein, 13 aa, Artificial Sequence
  Synthetic amino acid sequence
QGAIGSPGPAGPR

SEQ ID NO: 147 - protein, 49 aa, Artificial Sequence
  Synthetic amino acid sequence
GSEGSPGHPGQPGPPGPPGAPGPCCGGVGAAAIAGIGGEKAGGFAPYYG

SEQ ID NO: 148 - protein, 44 aa, Artificial Sequence
  Synthetic amino acid sequence
GPQGFQGNPGEPGEPGVSGPMGPRGPPGPPGKPGDDGEAGKPGK

SEQ ID NO: 149 - protein, 36 aa, Artificial Sequence
  Synthetic amino acid sequence
GAAGARGNDGQPGPAGPPGPVGPAGGPGFPGAPGAK

SEQ ID NO: 150 - protein, 35 aa, Artificial Sequence
  Synthetic amino acid sequence
AAGARGNDGQPGPAGPPGPVGPAGGPGFPGAPGAK

SEQ ID NO: 151 - protein, 22 aa, Artificial Sequence
  Synthetic amino acid sequence
PGAKGSAGAPGIAGAPGFPGPR

SEQ ID NO: 152 - protein, 18 aa, Artificial Sequence
  Synthetic amino acid sequence
GPRGPPGPQGATGPLGPK

SEQ ID NO: 153 - protein, 7 aa, Artificial Sequence
  Synthetic amino acid sequence
DGLAGPK

SEQ ID NO: 154 - protein, 26 aa, Artificial Sequence
  Synthetic amino acid sequence
PQGKVGPSGAPGEDGRPGPPGPQGAR

SEQ ID NO: 155 - protein, 14 aa, Artificial Sequence
  Synthetic amino acid sequence
GFPGPKGANGEPGK

SEQ ID NO: 156 - protein, 33 aa, Artificial Sequence
  Synthetic amino acid sequence
GLPGPPGPPGEGGKPGDQGVPGEAGAPGLVGPR

SEQ ID NO: 157 - protein, 27 aa, Artificial Sequence
  Synthetic amino acid sequence
GPPGEGGKPGDQGVPGEAGAPGLVGPR

SEQ ID NO: 158 - protein, 8 aa, Artificial Sequence
  Synthetic amino acid sequence
LQGMPGER

SEQ ID NO: 159 - protein, 20 aa, Artificial Sequence
  Synthetic amino acid sequence
GRGLTGPIGPPGPAGANGEK

SEQ ID NO: 160 - protein, 24 aa, Artificial Sequence
  Synthetic amino acid sequence
GLTGPIGPPGPAGANGEKGEVGPP

SEQ ID NO: 161 - protein, 31 aa, Artificial Sequence
  Synthetic amino acid sequence
GLTGPIGPPGPAGANGEKGEVGPPGPAGSAG

SEQ ID NO: 162 - protein, 32 aa, Artificial Sequence
  Synthetic amino acid sequence
LTGPIGPPGPAGANGEKGEVGPPGPAGSAGAR

SEQ ID NO: 163 - protein, 31 aa, Artificial Sequence
  Synthetic amino acid sequence
TGPIGPPGPAGANGEKGEVGPPGPAGSAGAR

SEQ ID NO: 164 - protein, 14 aa, Artificial Sequence
  Synthetic amino acid sequence
FAGPPGADGQPGAK

SEQ ID NO: 165 - protein, 13 aa, Artificial Sequence
  Synthetic amino acid sequence
AGPPGADGQPGAK

SEQ ID NO: 166 - protein, 22 aa, Artificial Sequence
  Synthetic amino acid sequence
SGPPGRAGEPGLQGPAGPPGEK

SEQ ID NO: 167 - protein, 21 aa, Artificial Sequence
  Synthetic amino acid sequence
GPPGRAGEPGLQGPAGPPGEK

SEQ ID NO: 168 - protein, 25 aa, Artificial Sequence
  Synthetic amino acid sequence
PPGLTGPAGEPGREGSPGADGPPGR

SEQ ID NO: 169 - protein, 18 aa, Artificial Sequence
  Synthetic amino acid sequence
PGPGIDMSAFAGLGPREK

SEQ ID NO: 170 - protein, 8 aa, Artificial Sequence
  Synthetic amino acid sequence
IEWHLNAF

SEQ ID NO: 171 - protein, 17 aa, Artificial Sequence
  Synthetic amino acid sequence
AITGPPTELITSEVTAR

SEQ ID NO: 172 - protein, 12 aa, Artificial Sequence
  Synthetic amino acid sequence
AIYAHTASEGLR

SEQ ID NO: 173 - protein, 10 aa, Artificial Sequence
  Synthetic amino acid sequence
LYDVTENSMR

SEQ ID NO: 174 - protein, 20 aa, Artificial Sequence
  Synthetic amino acid sequence
YLILYAPLTEGLAGDEKEMK

SEQ ID NO: 175 - protein, 13 aa, Artificial Sequence
  Synthetic amino acid sequence
YAPLTEGLAGDEK

SEQ ID NO: 176 - protein, 10 aa, Artificial Sequence
  Synthetic amino acid sequence
HVEMTSLCAH

SEQ ID NO: 177 - protein, 15 aa, Artificial Sequence
  Synthetic amino acid sequence
SIQGMPGMPGEKGEK

SEQ ID NO: 178 - protein, 10 aa, Artificial Sequence
  Synthetic amino acid sequence
QVCEQLIQSH

SEQ ID NO: 179 - protein, 35 aa, Artificial Sequence
  Synthetic amino acid sequence
EPGRPGSPGAPGEQGPPGTPGFPGNAGVPGTPGER

SEQ ID NO: 180 - protein, 15 aa, Artificial Sequence
  Synthetic amino acid sequence
GGSTNTGKAMTYVRE

SEQ ID NO: 181 - protein, 11 aa, Artificial Sequence
  Synthetic amino acid sequence
PKVMILITDGK

SEQ ID NO: 182 - protein, 17 aa, Artificial Sequence
  Synthetic amino acid sequence
PDDTHAYNVADFESLSR

SEQ ID NO: 183 - protein, 12 aa, Artificial Sequence
  Synthetic amino acid sequence
SVVEDEYSEPLK

SEQ ID NO: 184 - protein, 9 aa, Artificial Sequence
  Synthetic amino acid sequence
SETSTSLKD

SEQ ID NO: 185 - protein, 25 aa, Artificial Sequence
  Synthetic amino acid sequence
LKPDTPYTITVSSLYPDGEGGRMTG

SEQ ID NO: 186 - protein, 10 aa, Artificial Sequence
  Synthetic amino acid sequence
PGPAGGPGAK

SEQ ID NO: 187 - protein, 21 aa, Artificial Sequence
  Synthetic amino acid sequence
GRTGTPGLPGPPGPMGPPGDR

SEQ ID NO: 188 - protein, 22 aa, Artificial Sequence
  Synthetic amino acid sequence
TPGLPGPPGPMGPPGDRGFTGK

SEQ ID NO: 189 - protein, 21 aa, Artificial Sequence
  Synthetic amino acid sequence
GFPGTPGMQGPPGERGLPGEK

SEQ ID NO: 190 - protein, 7 aa, Artificial Sequence
  Synthetic amino acid sequence
QGPPGER

SEQ ID NO: 191 - protein, 15 aa, Artificial Sequence
  Synthetic amino acid sequence
PRGLPGPPGPQGESR

SEQ ID NO: 192 - protein, 23 aa, Artificial Sequence
  Synthetic amino acid sequence
PPSLGRPWAPLTGPSVPPPSSGR

SEQ ID NO: 193 - protein, 28 aa, Artificial Sequence
  Synthetic amino acid sequence
PGEDGKPGDTGPQGFPGTPGDVGPKGDK

SEQ ID NO: 194 - protein, 9 aa, Artificial Sequence
  Synthetic amino acid sequence
PGLPGEPGR

SEQ ID NO: 195 - protein, 20 aa, Artificial Sequence
  Synthetic amino acid sequence
GREGPPGFPGLPGPPGPPGR

SEQ ID NO: 196 - protein, 33 aa, Artificial Sequence
  Synthetic amino acid sequence
QDGSVLSVPGPEGRPGFAGFPGPAGPKGNLGSK

SEQ ID NO: 197 - protein, 21 aa, Artificial Sequence
  Synthetic amino acid sequence
AESSRPGPPGLPGNQGPPGPK

SEQ ID NO: 198 - protein, 9 aa, Artificial Sequence
  Synthetic amino acid sequence
GPPGPKGAK

SEQ ID NO: 199 - protein, 17 aa, Artificial Sequence
  Synthetic amino acid sequence
PGPPGPPGTMGASSGVR

SEQ ID NO: 200 - protein, 34 aa, Artificial Sequence
  Synthetic amino acid sequence
RLPEPQPYPGAPHHSSYVHLRPARPTSPPAHSHR

SEQ ID NO: 201 - protein, 16 aa, Artificial Sequence
  Synthetic amino acid sequence
LPEPQPYPGAPHHSSY

SEQ ID NO: 202 - protein, 9 aa, Artificial Sequence
  Synthetic amino acid sequence
NSPLSGGMR

SEQ ID NO: 203 - protein, 22 aa, Artificial Sequence
  Synthetic amino acid sequence
PSLGRPWAPLTGPSVPPPSSER

SEQ ID NO: 204 - protein, 24 aa, Artificial Sequence
  Synthetic amino acid sequence
GARGVSGFPGADGIPGHPGQGGPR

SEQ ID NO: 205 - protein, 19 aa, Artificial Sequence
  Synthetic amino acid sequence
GGPKGLPGLPGPPGPTGAK

SEQ ID NO: 206 - protein, 33 aa, Artificial Sequence
  Synthetic amino acid sequence
GPPGLHGFPGAPGQEGPLGLPGIPGREGLPGDR

SEQ ID NO: 207 - protein, 16 aa, Artificial Sequence
  Synthetic amino acid sequence
APGRPGSPGLPGMPGR

SEQ ID NO: 208 - protein, 14 aa, Artificial Sequence
  Synthetic amino acid sequence
LYCNPGDVCYYASR

SEQ ID NO: 209 - protein, 26 aa, Artificial Sequence
  Synthetic amino acid sequence
LMHTAAGDEGGGQSLVSPGSCLEDFR

SEQ ID NO: 210 - protein, 25 aa, Artificial Sequence
  Synthetic amino acid sequence
EPGPPGLPGSVGSPGVPGIGPPGAR

SEQ ID NO: 211 - protein, 30 aa, Artificial Sequence
  Synthetic amino acid sequence
PGVPGIGPPGARGPPGGQGPPGLSGPPGIK

SEQ ID NO: 212 - protein, 20 aa, Artificial Sequence
  Synthetic amino acid sequence
PPGGQGPPGLSGPPGIKGEK

SEQ ID NO: 213 - protein, 20 aa, Artificial Sequence
  Synthetic amino acid sequence
DPGFQGMPGIGGSPGITGSK

SEQ ID NO: 214 - protein, 28 aa, Artificial Sequence
  Synthetic amino acid sequence
KGQQGVTGLVGIPGPPGIPGFDGAPGQK

SEQ ID NO: 215 - protein, 10 aa, Artificial Sequence
  Synthetic amino acid sequence
SLLYVQGNER

SEQ ID NO: 216 - protein, 14 aa, Artificial Sequence
  Synthetic amino acid sequence
LFCNINNVCNFASR

SEQ ID NO: 217 - protein, 26 aa, Artificial Sequence
  Synthetic amino acid sequence
VMHTSAGAEGSGQALASPGSCLEEFR

SEQ ID NO: 218 - protein, 11 aa, Artificial Sequence
  Synthetic amino acid sequence
RSAPFIECHGR

SEQ ID NO: 219 - protein, 9 aa, Artificial Sequence
  Synthetic amino acid sequence
SFWLATIER

SEQ ID NO: 220 - protein, 7 aa, Artificial Sequence
  Synthetic amino acid sequence
WLATIER

SEQ ID NO: 221 - protein, 10 aa, Artificial Sequence
  Synthetic amino acid sequence
DGIPGPPGPK

SEQ ID NO: 222 - protein, 46 aa, Artificial Sequence
  Synthetic amino acid sequence
KGNPGYPGPPGIQGLPGPTGIPGPIGPPGPPGLMGPPGPPGLPGPK

SEQ ID NO: 223 - protein, 24 aa, Artificial Sequence
  Synthetic amino acid sequence
PHIPPSDEICEPGPPGPPGSPGDK

SEQ ID NO: 224 - protein, 18 aa, Artificial Sequence
  Synthetic amino acid sequence
GLPGLPGPPGSLGFPGQK

SEQ ID NO: 225 - protein, 11 aa, Artificial Sequence
  Synthetic amino acid sequence
PKGEPGGITFK

SEQ ID NO: 226 - protein, 20 aa, Artificial Sequence
  Synthetic amino acid sequence
TPGRIGLEGPPGPPGFPGPK

SEQ ID NO: 227 - protein, 15 aa, Artificial Sequence
  Synthetic amino acid sequence
GPPGRTGLDGLPGPK

SEQ ID NO: 228 - protein, 16 aa, Artificial Sequence
  Synthetic amino acid sequence
APGPIGPPGSPGLPGK

SEQ ID NO: 229 - protein, 21 aa, Artificial Sequence
  Synthetic amino acid sequence
KGEPGLPGPPGPMDPNLLGSK

SEQ ID NO: 230 - protein, 22 aa, Artificial Sequence
  Synthetic amino acid sequence
PGEPGPVGGGGHPGQPGPPGEK

SEQ ID NO: 231 - protein, 43 aa, Artificial Sequence
  Synthetic amino acid sequence
PALEGPKGNPGPQGPPGRPGPTGFQGLPGPEGPPGLPGNGGIK

SEQ ID NO: 232 - protein, 19 aa, Artificial Sequence
  Synthetic amino acid sequence
GPPGPPGLPGPSGQSIIIK

SEQ ID NO: 233 - protein, 11 aa, Artificial Sequence
  Synthetic amino acid sequence
VDGATGLPGMK

SEQ ID NO: 234 - protein, 34 aa, Artificial Sequence
  Synthetic amino acid sequence
KGQAGPPGVMGPPGPPGPPGPPGPGCTMGLGFED

SEQ ID NO: 235 - protein, 18 aa, Artificial Sequence
  Synthetic amino acid sequence
KLQLGELIPIPADSPPPP

SEQ ID NO: 236 - protein, 20 aa, Artificial Sequence
  Synthetic amino acid sequence
AWRTADTAVTGLASPLSTGK

SEQ ID NO: 237 - protein, 18 aa, Artificial Sequence
  Synthetic amino acid sequence
AVTGLASPLSTGKILDQK

SEQ ID NO: 238 - protein, 50 aa, Artificial Sequence
  Synthetic amino acid sequence
GVEDADEGALKEIASEPLNMHMFNLENFTSLHDIVGNLVSCVHSSVSPER

SEQ ID NO: 239 - protein, 11 aa, Artificial Sequence
  Synthetic amino acid sequence
NNLFTSSAGYR

SEQ ID NO: 240 - protein, 16 aa, Artificial Sequence
  Synthetic amino acid sequence
AAPLQGMLPGLLAPLR

SEQ ID NO: 241 - protein, 10 aa, Artificial Sequence
  Synthetic amino acid sequence
IGDLHPQIVN

SEQ ID NO: 242 - protein, 8 aa, Artificial Sequence
  Synthetic amino acid sequence
GPQGDQGR

SEQ ID NO: 243 - protein, 8 aa, Artificial Sequence
  Synthetic amino acid sequence
TDPAHDVR

SEQ ID NO: 244 - protein, 16 aa, Artificial Sequence
  Synthetic amino acid sequence
FSDGNSQGATPAAIEK

SEQ ID NO: 245 - protein, 8 aa, Artificial Sequence
  Synthetic amino acid sequence
QVNEPHIR

SEQ ID NO: 246 - protein, 9 aa, Artificial Sequence
  Synthetic amino acid sequence
GVFHQTVSR

SEQ ID NO: 247 - protein, 23 aa, Artificial Sequence
  Synthetic amino acid sequence
GPGLLLLAVQCLGTAVPSTGASK

SEQ ID NO: 248 - protein, 11 aa, Artificial Sequence
  Synthetic amino acid sequence
ALVCTCYGGSR

SEQ ID NO: 249 - protein, 13 aa, Artificial Sequence
  Synthetic amino acid sequence
MVDCTCLGEGSGR

SEQ ID NO: 250 - protein, 16 aa, Artificial Sequence
  Synthetic amino acid sequence
AAHEEICTTNEGVMYR

SEQ ID NO: 251 - protein, 16 aa, Artificial Sequence
  Synthetic amino acid sequence
SHPIQWNAPQPSHISK

SEQ ID NO: 252 - protein, 15 aa, Artificial Sequence
  Synthetic amino acid sequence
VVSWVSASDTVSGFR

SEQ ID NO: 253 - protein, 19 aa, Artificial Sequence
  Synthetic amino acid sequence
SDTVPSPRDLQFVEVTDVK

SEQ ID NO: 254 - protein, 15 aa, Artificial Sequence
  Synthetic amino acid sequence
VDVIPVNLPGEHGQR

SEQ ID NO: 255 - protein, 20 aa, Artificial Sequence
  Synthetic amino acid sequence
VFAVSHGRESKPLTAQQTTK

SEQ ID NO: 256 - protein, 13 aa, Artificial Sequence
  Synthetic amino acid sequence
LGVRPSQGGEAPR

SEQ ID NO: 257 - protein, 19 aa, Artificial Sequence
  Synthetic amino acid sequence
DAPIVNKVVTPLSPPTNLH

SEQ ID NO: 258 - protein, 8 aa, Artificial Sequence
  Synthetic amino acid sequence
TPDITGYR

SEQ ID NO: 259 - protein, 22 aa, Artificial Sequence
  Synthetic amino acid sequence
PGTEYVVSVSSVYEQHESTPLR

SEQ ID NO: 260 - protein, 22 aa, Artificial Sequence
  Synthetic amino acid sequence
TGLDSPTGIDFSDITANSFTVH

SEQ ID NO: 261 - protein, 8 aa, Artificial Sequence
  Synthetic amino acid sequence
TVHWIAPR

SEQ ID NO: 262 - protein, 12 aa, Artificial Sequence
  Synthetic amino acid sequence
SPVQEFTVPGSK

SEQ ID NO: 263 - protein, 28 aa, Artificial Sequence
  Synthetic amino acid sequence
VVSVYAQNPSGESQPLVQTAVTNIDRPK

SEQ ID NO: 264 - protein, 37 aa, Artificial Sequence
  Synthetic amino acid sequence
RPGSEYTVSVVALHDDMESQPLIGTQSTAIPAPTDLK

SEQ ID NO: 265 - protein, 9 aa, Artificial Sequence
  Synthetic amino acid sequence
YEVSVYALK

SEQ ID NO: 266 - protein, 11 aa, Artificial Sequence
  Synthetic amino acid sequence
IYLYTLNDNAR

SEQ ID NO: 267 - protein, 10 aa, Artificial Sequence
  Synthetic amino acid sequence
SLLVSWQPPR

SEQ ID NO: 268 - protein, 9 aa, Artificial Sequence
  Synthetic amino acid sequence
YEKPGSPPR

SEQ ID NO: 269 - protein, 8 aa, Artificial Sequence
  Synthetic amino acid sequence
TPFVTHPG

SEQ ID NO: 270 - protein, 11 aa, Artificial Sequence
  Synthetic amino acid sequence
TPFVTHPGYDT

SEQ ID NO: 271 - protein, 31 aa, Artificial Sequence
  Synthetic amino acid sequence
TPFVTHPGYDTGNGIQLPGTSGQQPSVGQQM

SEQ ID NO: 272 - protein, 23 aa, Artificial Sequence
  Synthetic amino acid sequence
QDTSEYIISCHPVGTDEEPLQFR

SEQ ID NO: 273 - protein, 27 aa, Artificial Sequence
  Synthetic amino acid sequence
VPGTSTSATLTGLTRGATYNIIVEALK

SEQ ID NO: 274 - protein, 10 aa, Artificial Sequence
  Synthetic amino acid sequence
VREEVVTVGN

SEQ ID NO: 275 - protein, 31 aa, Artificial Sequence
  Synthetic amino acid sequence
SVNEGLNQPTDDSCFDPYTVSHYAVGDEWER

SEQ ID NO: 276 - protein, 9 aa, Artificial Sequence
  Synthetic amino acid sequence
LGFGSGHFR

SEQ ID NO: 277 - protein, 27 aa, Artificial Sequence
  Synthetic amino acid sequence
ACEDIDECSLPNICVFGTCHNLPGLFR

SEQ ID NO: 278 - protein, 11 aa, Artificial Sequence
  Synthetic amino acid sequence
TGLPVDIDECR

SEQ ID NO: 279 - protein, 8 aa, Artificial Sequence
  Synthetic amino acid sequence
PVDIDECR

SEQ ID NO: 280 - protein, 18 aa, Artificial Sequence
  Synthetic amino acid sequence
EIPGVCNGVCINHVGSFR

SEQ ID NO: 281 - protein, 19 aa, Artificial Sequence
  Synthetic amino acid sequence
EIPGVCENGVCINMVGSFR

SEQ ID NO: 282 - protein, 18 aa, Artificial Sequence
  Synthetic amino acid sequence
LLVCEDIDECQNGPVCQR

SEQ ID NO: 283 - protein, 13 aa, Artificial Sequence
  Synthetic amino acid sequence
TCVDINECLLEPR

SEQ ID NO: 284 - protein, 20 aa, Artificial Sequence
  Synthetic amino acid sequence
GEGWGDPCELCPTEPDEAFR

SEQ ID NO: 285 - protein, 14 aa, Artificial Sequence
  Synthetic amino acid sequence
CTDYTAECKPQVTR

SEQ ID NO: 286 - protein, 11 aa, Artificial Sequence
  Synthetic amino acid sequence
IYISGMAPRPS

SEQ ID NO: 287 - protein, 15 aa, Artificial Sequence
  Synthetic amino acid sequence
TCEPIQSVFFFSGDK

SEQ ID NO: 288 - protein, 14 aa, Artificial Sequence
  Synthetic amino acid sequence
SIAQYWLGCPAPGH

SEQ ID NO: 289 - protein, 10 aa, Artificial Sequence
  Synthetic amino acid sequence
WLGCPAPGHL

SEQ ID NO: 290 - protein, 8 aa, Artificial Sequence
  Synthetic amino acid sequence
CNSDLVIR

SEQ ID NO: 291 - protein, 11 aa, Artificial Sequence
  Synthetic amino acid sequence
LQDGLLHITTC

SEQ ID NO: 292 - protein, 14 aa, Artificial Sequence
  Synthetic amino acid sequence
SFVAPWNSLSLAQR

SEQ ID NO: 293 - protein, 10 aa, Artificial Sequence
  Synthetic amino acid sequence
SDDGWVNLNR

SEQ ID NO: 294 - protein, 14 aa, Artificial Sequence
  Synthetic amino acid sequence
SYQCPQGQVIVAVR

SEQ ID NO: 295 - protein, 15 aa, Artificial Sequence
  Synthetic amino acid sequence
SLGEPTECWWEEINR

SEQ ID NO: 296 - protein, 12 aa, Artificial Sequence
  Synthetic amino acid sequence
SNNGLVAGFQSR

SEQ ID NO: 297 - protein, 14 aa, Artificial Sequence
  Synthetic amino acid sequence
VASNLNLKPGECLR

SEQ ID NO: 298 - protein, 11 aa, Artificial Sequence
  Synthetic amino acid sequence
GDANTIVCNSK

SEQ ID NO: 299 - protein, 8 aa, Artificial Sequence
  Synthetic amino acid sequence
MAADGDFK

SEQ ID NO: 300 - protein, 21 aa, Artificial Sequence
  Synthetic amino acid sequence
CAPECNCPESYPSAMYCDELK

SEQ ID NO: 301 - protein, 11 aa, Artificial Sequence
  Synthetic amino acid sequence
RNNQIDHIDEK

SEQ ID NO: 302 - protein, 9 aa, Artificial Sequence
  Synthetic amino acid sequence
NNQIDHIDE

SEQ ID NO: 303 - protein, 11 aa, Artificial Sequence
  Synthetic amino acid sequence
ILDHNLLENSK

SEQ ID NO: 304 - protein, 9 aa, Artificial Sequence
  Synthetic amino acid sequence
SLEDLQLTH

SEQ ID NO: 305 - protein, 7 aa, Artificial Sequence
  Synthetic amino acid sequence
IHLQHNR

SEQ ID NO: 306 - protein, 11 aa, Artificial Sequence
  Synthetic amino acid sequence
CKILGPLSYSK

SEQ ID NO: 307 - protein, 8 aa, Artificial Sequence
  Synthetic amino acid sequence
EVPSALPR

SEQ ID NO: 308 - protein, 9 aa, Artificial Sequence
  Synthetic amino acid sequence
RLSQNHISR

SEQ ID NO: 309 - protein, 17 aa, Artificial Sequence
  Synthetic amino acid sequence
RLSQNHISRIPPGVFSK

SEQ ID NO: 310 - protein, 10 aa, Artificial Sequence
  Synthetic amino acid sequence
LSDGVFKPDT

SEQ ID NO: 311 - protein, 8 aa, Artificial Sequence
  Synthetic amino acid sequence
NLAHNILR

SEQ ID NO: 312 - protein, 7 aa, Artificial Sequence
  Synthetic amino acid sequence
LAHNILR

SEQ ID NO: 313 - protein, 24 aa, Artificial Sequence
  Synthetic amino acid sequence
LDSNKIETIPNGYFKSFPNLAFIR

SEQ ID NO: 314 - protein, 16 aa, Artificial Sequence
  Synthetic amino acid sequence
IETIPNGYFKSFPNLA

SEQ ID NO: 315 - protein, 13 aa, Artificial Sequence
  Synthetic amino acid sequence
SFPNLAFIRLNYN

SEQ ID NO: 316 - protein, 8 aa, Artificial Sequence
  Synthetic amino acid sequence
LNNNSIEK

SEQ ID NO: 317 - protein, 19 aa, Artificial Sequence
  Synthetic amino acid sequence
DLVAFHDFSSDLENVPHLR

SEQ ID NO: 318 - protein, 11 aa, Artificial Sequence
  Synthetic amino acid sequence
ELEPGVEYFIR

SEQ ID NO: 319 - protein, 12 aa, Artificial Sequence
  Synthetic amino acid sequence
TVSIYGVIQGYR

SEQ ID NO: 320 - protein, 9 aa, Artificial Sequence
  Synthetic amino acid sequence
TVTLHGEVR

SEQ ID NO: 321 - protein, 22 aa, Artificial Sequence
  Synthetic amino acid sequence
FRITYVPITGGTPSMVTVDGTK

SEQ ID NO: 322 - protein, 24 aa, Artificial Sequence
  Synthetic amino acid sequence
WRPQPPAEGPGGELTVPGTTRTVS

SEQ ID NO: 323 - protein, 9 aa, Artificial Sequence
  Synthetic amino acid sequence
FDSFTVQYK

SEQ ID NO: 324 - protein, 15 aa, Artificial Sequence
  Synthetic amino acid sequence
GEESEVTVGGLEPGR

SEQ ID NO: 325 - protein, 7 aa, Artificial Sequence
  Synthetic amino acid sequence
EPPNKPR

SEQ ID NO: 326 - protein, 21 aa, Artificial Sequence
  Synthetic amino acid sequence
GFEESEPLTGFLTTVPDGPTQ

SEQ ID NO: 327 - protein, 8 aa, Artificial Sequence
  Synthetic amino acid sequence
ISCTIANR

SEQ ID NO: 328 - protein, 18 aa, Artificial Sequence
  Synthetic amino acid sequence
LFSDGNSQGATPAAIEKA

SEQ ID NO: 329 - protein, 11 aa, Artificial Sequence
  Synthetic amino acid sequence
RGVFHQTVSRK

SEQ ID NO: 330 - protein, 10 aa, Artificial Sequence
  Synthetic amino acid sequence
RQVNEPHIRV

SEQ ID NO: 331 - protein, 10 aa, Artificial Sequence
  Synthetic amino acid sequence
RTDPAHDVRV

SEQ ID NO: 332 - protein, 7 aa, Artificial Sequence
  Synthetic amino acid sequence
DAPIVNK

SEQ ID NO: 333 - protein, 7 aa, Artificial Sequence
  Synthetic amino acid sequence
SEPLIGR

SEQ ID NO: 334 - protein, 7 aa, Artificial Sequence
  Synthetic amino acid sequence
ATITGYR

SEQ ID NO: 335 - protein, 7 aa, Artificial Sequence
  Synthetic amino acid sequence
AQITGYR

SEQ ID NO: 336 - protein, 8 aa, Artificial Sequence
  Synthetic amino acid sequence
SDTVPSPR

SEQ ID NO: 337 - protein, 8 aa, Artificial Sequence
  Synthetic amino acid sequence
VFAVSHGR

SEQ ID NO: 338 - protein, 9 aa, Artificial Sequence
  Synthetic amino acid sequence
ISCTIANRC

SEQ ID NO: 339 - protein, 9 aa, Artificial Sequence
  Synthetic amino acid sequence
PLTAQQTTK

SEQ ID NO: 340 - protein, 9 aa, Artificial Sequence
  Synthetic amino acid sequence
YEVSVYALK

SEQ ID NO: 341 - protein, 10 aa, Artificial Sequence
  Synthetic amino acid sequence
QYNVGPSVSK

SEQ ID NO: 342 - protein, 12 aa, Artificial Sequence
  Synthetic amino acid sequence
GATYNIIVEALK

SEQ ID NO: 343 - protein, 11 aa, Artificial Sequence
  Synthetic amino acid sequence
DLQFVEVTDVK

SEQ ID NO: 344 - protein, 13 aa, Artificial Sequence
  Synthetic amino acid sequence
LGVRPSQGGEAPR

SEQ ID NO: 345 - protein, 11 aa, Artificial Sequence
  Synthetic amino acid sequence
IYLYTLNDNAR

SEQ ID NO: 346 - protein, 15 aa, Artificial Sequence
  Synthetic amino acid sequence
VPGTSTSATLTGLTR

SEQ ID NO: 347 - protein, 15 aa, Artificial Sequence
  Synthetic amino acid sequence
VDVIPVNLPGEHGQR

SEQ ID NO: 348 - protein, 29 aa, Artificial Sequence
  Synthetic amino acid sequence
KACEDIDECSLPNICVFGTCHNLPGLFRC

SEQ ID NO: 349 - protein, 13 aa, Artificial Sequence
  Synthetic amino acid sequence
YTGLPVDIDECRE

SEQ ID NO: 350 - protein, 10 aa, Artificial Sequence
  Synthetic amino acid sequence
LPVDIDECRE

SEQ ID NO: 351 - protein, 21 aa, Artificial Sequence
  Synthetic amino acid sequence
REIPGVCENGVCINMVGSFRC

SEQ ID NO: 352 - protein, 20 aa, Artificial Sequence
  Synthetic amino acid sequence
KLLVCEDIDECQNGPVCQRN

SEQ ID NO: 353 - protein, 15 aa, Artificial Sequence
  Synthetic amino acid sequence
RTCVDINECLLEPRK

SEQ ID NO: 354 - protein, 22 aa, Artificial Sequence
  Synthetic amino acid sequence
KGEGWGDPCELCPTEPDEAFRQ

SEQ ID NO: 355 - protein, 8 aa, Artificial Sequence
  Synthetic amino acid sequence
NPGAPGPR

(19) United States
(12) Patent Application Publication
(10) Pub. No.: US 2015/0160201 A1
(43) Pub. Date: Jun. 11, 2015

(54) Compositions and Methods for Detecting Unstable Arteriosclerotic Plaques

(71) Applicant: The Regents of the University of California, Oakland, CA (US)

(72) Inventors: John P. Kane, Hillsborough, CA (US); Robert J. Chalkley, San Francisco, CA (US); Olga Miroshnychenko, Palo Alto, CA (US)

(21) Appl. No.: 14/391,153

(22) PCT Filed: Mar. 6, 2013
(86) PCT No.: PCT/US13/29276

§ 371 Date: Oct. 7, 2014

Related U.S. Application Data
(60) Provisional application No. 61/635,645, filed on Apr. 19, 2012.

(51) Int. Cl. G01N 33/53

(52) U.S. Cl. CPC G01N 33/5308; G01N 2800/323

(57) ABSTRACT

The present disclosure provides methods of detecting an unstable arteriosclerotic plaque in an individual, involving detecting in a biological sample from the individual an enzymatic cleavage product of a protein component of an arteriosclerotic plaque. The present disclosure provides methods of assessing the risk that an individual will develop an occlusive vascular event. The present disclosure further provides kits for carrying out a subject method.

### CROSS-REFERENCE

This application claims the benefit of U.S. Provisional Patent Application No. 61/635,645, filed Apr. 19, 2012, which application is incorporated herein by reference in its entirety.

### STATEMENT REGARDING FEDERALLY SPONSORED RESEARCH

This invention was made with government support under Grant No. P41 RR001614 awarded by the National Institutes of Health, National Center for Research Resources. The government has certain rights in the invention.

### INTRODUCTION

Cardiovascular disease (CVD) is the general term for heart and blood vessel diseases, including atherosclerosis, coronary artery disease, cerebrovascular disease, aorto-iliac disease, and peripheral vascular disease. Individuals with CVD may develop a number of complications, such as myocardial infarction, stroke, angina pectoris, transient ischemic attacks, congestive heart failure, aortic aneurysm, and death.

Arterial plaque instability is a critical element in occlusive vascular disease events, including myocardial ischemia, myocardial infarction, stroke, and peripheral arterial disease. As plaques become unstable, they erode or rupture, exposing prothrombotic stimuli to the blood, which in turn initiates thrombi.

Levels of the acute phase reactant C-reactive protein (CRP) have been used to estimate an individual's risk of developing a cardiovascular disorder. However, CRP may be found in the blood of individuals with inflammation due to causes other than CVD; as such, the value of CRP as a diagnostic or prognostic tool is limited.

There is a need in the art for methods of determining an individual's risk of developing an occlusive vascular event.

### LITERATURE

U.S. Patent Publication No. 2010/0323377; Libby et al. (2010) Circ. J. 74:213; Galis et al. (1994) J. Clin. Invest. 94:2493; Skølt-Arkil et al. (2010) Assay and Drug Development Technologies 8:542; Barascuk et al. (2010) BMC Cardiovasc. Dis. 10:19; Libby (2006) Arterioscler. Thromb. Vasc. Biol. 26:2181.

### SUMMARY

The present disclosure provides methods of detecting an unstable arteriosclerotic plaque in an individual, involving detecting in a biological sample from the individual an enzymatic cleavage product of a protein component of an arteriosclerotic plaque. The present disclosure provides methods of assessing the risk that an individual will develop an occlusive vascular event. The present disclosure further provides kits for carrying out a subject method.

In a first embodiment, the present disclosure provides a method for detecting an unstable arteriosclerotic plaque in an individual, the method comprising detecting in a biological sample from the individual an enzymatic cleavage product of a protein component of an arteriosclerotic plaque. In some cases, the protein component is not collagen type III, elastin, decorin, biglycan, versican, apolipoprotein E, C-reactive protein, or lumican.

In the first embodiment of a subject method, the protein can be a structural protein, e.g., a non-enzymatic protein. In some cases, in the first and/or the second embodiment of a subject method, the individual is asymptomatic with respect to an arterial occlusive event and/or is an apparently healthy human subject. In some cases, in any of the above embodiments of a subject method, the individual has experienced one or more typical symptoms of cardiovascular disease. In some cases, in any of the above embodiments of a subject method, the individual has experienced an atypical symptom of cardiovascular disease. In any of the above embodiments, the biological sample is blood; or is a blood fraction (e.g., serum or plasma). In any of the above embodiments, level of the one or more enzymatic cleavage products is determined by an immunological method. In any of the above embodiments, the protein component is fibrillin, vitronectin, fibronectin, tenascin, prolargin, dermatopontin, vascular collagen, metalloproteinase inhibitor-1, galectin-1, or tenascin-X. For example, the collagen can be collagen alpha-1 (I) chain, collagen alpha-1 (II) chain, collagen alpha-1 (IV) chain, collagen alpha-1 (VI) chain, collagen alpha-1 (XII), collagen alpha-1 (XIV) chain, collagen alpha-1 (XV) chain, collagen alpha-1 (XVIII), collagen alpha-1 (XIX); collagen alpha-2 (I) chain, collagen alpha-3 (VI), collagen alpha-2 (IV), or collagen alpha-5 (IV). In any of the above embodiments, the enzymatic cleavage product has a molecular weight in a range of from about 0.5 kDa to about 50 kDa; for example, the enzymatic cleavage product can have a length in a range of from about 5 amino acids to about 500 amino acids.

In any of the above embodiments of a subject method for detecting an unstable arteriosclerotic plaque in an individual, the detecting step can comprise processing the enzymatic cleavage product in vitro. For example, in some cases, the processing comprises trypsin digestion.

In any of the above embodiments of a subject method for detecting an unstable arteriosclerotic plaque in an individual, the enzymatic cleavage product can be a cleavage product of a matrix metalloproteinase (MMP). For example, in some cases, the MMP is secreted by a macrophage. For example, in some cases, the MMP is MMP1, MMP2, MMP3, MMP7, MMP8, MMP9, MMP10, MMP11, MMP12, or MMP13.

In some cases, the enzymatic cleavage product is a cleavage product of a cathepsin.

In any of the above embodiments of a subject method for detecting an unstable arteriosclerotic plaque in an individual, the method can further comprise generating a report providing an indication of the risk that the individual will experience an occlusive vascular event.

In a second embodiment, the present disclosure provides a method for determining a risk that an individual will develop an occlusive vascular event, the method comprising determining the level, in a biological sample from the individual, of an enzymatic cleavage product of a protein component of an arteriosclerotic plaque, wherein a level of the enzymatic cleavage product that is higher than a normal control level indicates risk of developing an occlusive vascular event. For example, the protein component can be fibrillin, vitronectin, fibronectin, tenascin, prolargin, dermatopontin, vascular collagen, metalloproteinase inhibitor-1, galectin-1, or tenascin-X. In some cases, the level of the one or more enzymatic cleavage products is determined by an immunological method. In some cases, the protein component is not collagen type III, elastin, decorin, biglycan, versican, apolipoprotein E, C-reactive protein, or lumican.

In any of the above embodiments of a subject method for determining a risk that an individual will develop an occlusive vascular event, the biological sample can be blood, serum, or plasma. In any of the above embodiments of a subject method for determining a risk that an individual will develop an occlusive vascular event, the subject can be an apparently healthy human subject. In any of the above embodiments of a subject method for determining a risk that an individual will develop an occlusive vascular event, the individual can be one who does not have a history of having an occlusive vascular event.

In a third embodiment, the present disclosure provides a kit for detecting an unstable arteriosclerotic plaque in an individual, the kit comprising: a) a binding reagent that specifically binds an enzymatic cleavage product of a protein component of an arteriosclerotic plaque; and b) a control that provides for quantitation of the enzymatic product. In some cases, the protein component is not collagen type III, elastin, decorin, biglycan, versican, apolipoprotein E, C-reactive protein, or lumican.

In some embodiments of a subject kit, the reagent that specifically binds an enzymatic cleavage product of a protein component of an arteriosclerotic plaque is an antibody. For example, the antibody can be a monoclonal antibody, or an antigen-binding fragment. In some embodiments of a subject kit, the antibody is immobilized on an insoluble support. In some embodiments of a subject kit, the antibody comprises a detectable label; in some of these embodiments, the kit further comprises one or more reagents for developing a detectable label.

In a fourth embodiment, the present disclosure provides an assay device for use in detecting, in a liquid biological sample obtained from an individual, an enzymatic cleavage product of a protein component of an arteriosclerotic plaque, the device comprising a matrix defining an axial flow path, the matrix comprising: i) a sample receiving zone at an upstream end of the flow path that receives the liquid sample; ii) one or more test zones positioned within the flow path and downstream from the sample receiving zone, each of said one or more test zones comprising an antibody specific for an enzymatic cleavage product of a protein component of an arteriosclerotic plaque immobilized in each of said test zones, wherein each of said immobilized antibodies is capable of binding different enzymatic cleavage product present in said liquid sample, to form an immobilized antibody/enzymatic cleavage product complex; and iii) one or more control zones positioned within the flow path and downstream from the sample receiving zone. In some embodiments, the protein component is not collagen type III, elastin, decorin, biglycan, versican, apolipoprotein E, C-reactive protein, or lumican.

In some embodiments of a subject assay device, the one or more control zones are positioned between the test zones when two or more test zones are present. In some embodiments of a subject assay device, wherein the test zones and control zones are positioned in an alternating format within the flow path beginning with a test zone positioned upstream of any control zone. In some embodiments of a subject assay device, the assay device further comprises a label zone positioned upstream of a test zone, wherein the label zone comprises a labeled antibody specific for an enzymatic cleavage product of a protein component of an arteriosclerotic plaque, wherein the labeled antibody is capable of binding an enzymatic cleavage product present in an immobilized antibody/enzymatic cleavage product complex to form a labeled immobilized antibody/enzymatic cleavage product complex, and wherein the labeled antibody is mobilizable in the presence of the liquid sample.

In some embodiments of a subject assay device that include a labeled antibody, the labeled antibody comprises a label component selected from the group consisting of a chemiluminescent agent, a particulate label, a colorimetric agent, an energy transfer agent, an enzyme, a fluorescent agent, and a radioisotope. In some embodiments of a subject assay device, the matrix is positioned within a housing comprising a support and optionally a cover, wherein the housing contains an application aperture and one or more observation ports. In any of the embodiments of a subject assay device, the device can be a test strip or a dipstick assay device. In any of the embodiments of a subject assay device, the liquid sample can be blood, serum, or plasma.

### BRIEF DESCRIPTION OF THE DRAWINGS

FIGS. 1A-B provide an amino acid sequence of alpha-1 collagen (type I).

FIGS. 2A-B provide an amino acid sequence of alpha-1 collagen (type II).

FIGS. 3A-B provide an amino acid sequence of alpha-1 collagen (type III).

FIGS. 4A-B provide an amino acid sequence of alpha-1 collagen (type IV).

FIG. 5 provides an amino acid sequence of alpha-1 collagen (type VI).

FIGS. 6A-C provide an amino acid sequence of alpha-3 collagen (type VI).

FIGS. 7A-C provide an amino acid sequence of alpha-1 collagen (type XII).

FIGS. 8A-B provide an amino acid sequence of alpha-1 collagen (type XIV).

FIGS. 9A-B provide an amino acid sequence of alpha-1 collagen (type XV).

FIGS. 10A-B provide an amino acid sequence of alpha-1 collagen (type XVIII).

FIG. 11 provides an amino acid sequence of alpha-1 collagen (type XIX).

FIGS. 12A-B provide an amino acid sequence of alpha-2 collagen (type I).

FIGS. 13A-C provide an amino acid sequence of fibronectin.

FIGS. 14A-C provide an amino acid sequence of fibrillin-1.

FIG. 15 provides an amino acid sequence of dermatopontin.

FIG. 16 provides an amino acid sequence of metalloproteinase inhibitor-1.

FIG. 17 provides an amino acid sequence of galectin-1.

FIG. 18 provides an amino acid sequence of lumican.

FIG. 19 provides an amino acid sequence of prolargin.

FIGS. 20A and B provide an amino acid sequence of tenascin.

FIG. 21 provides an amino acid sequence of vitronectin.

FIGS. 22A-D provide an amino acid sequence of tenascin-X.

FIGS. 23A and 23B provide an amino acid sequence of collagen alpha-2(IV).

FIGS. 24A and 24B provide an amino acid sequence of collagen alpha-5(IV).

FIG. 25 provides an amino acid sequence of elastin.

### DEFINITIONS

The terms “polypeptide,” “peptide” and “protein”, used interchangeably herein, refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like. NH₂ refers to the free amino group present at the amino terminus of a polypeptide. COOH refers to the free carboxyl group present at the carboxyl terminus of a polypeptide. In keeping with standard polypeptide nomenclature, J. Biol. Chem., 243 (1969), 3552-59 is used.

A “biological sample” encompasses a variety of sample types obtained from an individual and can be used in a diagnostic or monitoring assay. The definition encompasses blood, blood products, and other liquid samples of biological origin; and solid tissue samples such as a biopsy specimen. The definition includes biological samples obtained via catheter during or as a result of coronary angiogram; and biological samples obtained during catheterization of a carotid artery, a femoral artery, or the aorta. The definition also includes samples that have been manipulated in any way after their procurement, such as by treatment with reagents, solubilization, or enrichment for certain components, such as peptides (e.g., enzymatic cleavage products of an arteriosclerotic plaque). The term “biological sample” encompasses a clinical sample, and also includes serum, plasma, biological fluid, and tissue samples. Enzymatic cleavage products of an arteriosclerotic plaque present in a biological sample can be eluted, monomerized, solubilized, etc., or otherwise treated in order to render the enzymatic cleavage products in a physical state suitable for analysis. Enzymatic cleavage products of an arteriosclerotic plaque present in a biological sample can be purified from the liquid sample, e.g., using immunoaffinity methods. For example, magnetic beads comprising an antibody specific for a given enzymatic cleavage product can be used to enrich the cleavage product from a biological sample.

The terms “individual,” “subject,” “host,” and “patient,” used interchangeably herein, refer to a mammal, including, e.g., humans and non-human primates.

“Conservative amino acid substitution” refers to a substitution of one amino acid residue for another sharing chemical and physical properties of the amino acid side chain (e.g., charge, size, hydrophobicity/hydrophilicity). “Conservative substitutions” are intended to include substitution within the following groups of amino acid residues: gly, ala; val, ile, leu; asp, glu; asn, gln; ser, thr; lys, arg; and phe, tyr. Guidance for such substitutions can be drawn from alignments of amino acid sequences of polypeptides.

“Isolated” refers to an entity of interest that is in an environment different from that in which the compound may naturally occur. “Isolated” is meant to include compounds that are within samples that are substantially enriched for the compound of interest and/or in which the compound of interest is partially or substantially purified.

By “purified” is meant a compound of interest (e.g., a polypeptide) has been separated from components that accompany it in nature. “Purified” can also be used to refer to a compound of interest separated from components that can accompany it during manufacture (e.g., in chemical synthesis). In some embodiments, a compound is substantially pure when it is at least 50% to 60%, by weight, free from organic molecules with which it is naturally associated or with which it is associated during manufacture. In some embodiments, the preparation is at least 75%, at least 90%, at least 95%, or at least 99%, by weight, of the compound of interest. A substantially pure compound can be obtained, for example, by extraction from a natural source (e.g., bacteria), by chemically synthesizing a compound, or by a combination of purification and chemical modification. A substantially pure compound can also be obtained by, for example, enriching a sample that contains the compound. Purity can be measured by any appropriate method, e.g., chromatography, mass spectroscopy, high performance liquid chromatography analysis, etc.

“Axial flow” as used herein refers to lateral, vertical or transverse flow through a particular matrix or material comprising one or more test and/or control zones. The type of flow contemplated in a particular device, assay or method varies according to the structure of the device. Without being bound by theory, lateral, vertical or transverse flow may refer to flow of a fluid sample from the point of fluid contact on one end or side of a particular matrix (the upstream or proximal end) to an area downstream (or distal) of this contact. The downstream area may be on the same side or on the opposite side of the matrix from the point of fluid contact. For example, in vertical flow devices of the present invention, axial flow may progress vertically from and through a first member (top to bottom) to a second member and from there on to an absorbent medium. By way of further example, and as will be appreciated by those of skill in the art, in a vertical flow device configured, for example, as a dipstick, a fluid sample may flow literally up the device, in which case however, the point of first contact of the fluid sample to the device is nonetheless considered the upstream (i.e., proximal) end and the point of termination of flow the downstream (i.e., distal) end.

As used herein the terms “upstream” and “downstream” refer to the direction of fluid sample flow subsequent to contact of the fluid sample with a representative device of the present disclosure, wherein, under normal operating conditions, the fluid sample flow direction runs from an upstream position to a downstream position. For example, when fluid sample is initially contacted with the sample receiving zone, the fluid sample then flows downstream through the label zone and so forth.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “an enzymatic cleavage product” includes a plurality of such enzymatic cleavage products and reference to “the protease secreted by an inflammatory cell” includes reference to one or more such proteases and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed, to the extent that such combinations embrace subject matter that are, for example, compounds that are stable compounds (i.e., compounds that can be made, isolated, characterized, and tested for biological activity). In addition, all sub-combinations of the various embodiments and elements thereof (e.g., elements of the chemical groups listed in the embodiments describing such variables) are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides methods of detecting an unstable arteriosclerotic plaque in an individual, the methods generally involving detecting in a biological sample from the individual an enzymatic cleavage product of a protein component of an arteriosclerotic plaque. The present disclosure provides methods of assessing the risk that an individual will develop an occlusive vascular event. The present disclosure further provides kits for carrying out a subject method.

Stable arteriosclerotic plaques can comprise extracellular matrix (ECM) components. Under certain circumstances, inflammatory cells secrete enzymes that can break down a protein component of an arteriosclerotic plaque. As plaques become unstable, they erode or rupture, exposing prothrombotic stimuli to the blood, which in turn initiates thrombi. Thus, arterial plaque instability is a critical element in occlusive vascular disease events, including myocardial ischemia, myocardial infarction, stroke, and peripheral arterial disease. A hallmark of an unstable arteriosclerotic plaque (also referred to as an “unstable atherosclerotic plaque”) is the presence in an arteriosclerotic plaque of inflammatory cells, which cells secrete enzymes that proteolytically cleave protein components of the plaque, thereby destabilizing the plaque. The present disclosure provides methods of detecting an unstable arteriosclerotic plaque. Detection of an unstable arteriosclerotic plaque can provide an indication of an individual's risk of developing an occlusive vascular event. Thus, the present disclosure provides methods of determining a risk that an individual will develop an occlusive vascular event. Based on detection an enzymatic cleavage product of a protein component of an arteriosclerotic plaque, a physician or other qualified medical personnel can determine whether appropriate medical intervention is advised, e.g., in order to reduce the risk that an occlusive vascular event will actually occur.

### Methods of Detecting an Unstable Arteriosclerotic Plaque

The present disclosure provides methods of detecting an unstable arteriosclerotic plaque in an individual, the methods generally involving detecting in a biological sample from the individual an enzymatic cleavage product of a protein component of an arteriosclerotic plaque. The enzymatic cleavage products are generated in vivo by enzymes produced by cells in the vasculature.

Protein components of an arteriosclerotic plaque include non-enzymatic proteins. Protein components of an arteriosclerotic plaque include structural proteins.

Enzymatic cleavage products of a protein component of an arteriosclerotic plaque include cleavage products generated by an enzyme produced by a cell in the vasculature (e.g., cleavage products generated in vivo in the vasculature by enzyme(s) produced by a cell (e.g., an inflammatory cell) in the vasculature). Enzymatic cleavage products include unmodified polypeptides and covalently modified polypeptides. Covalently modified polypeptides include polypeptides comprising a covalently linked chemical adduct. For example, a covalently modified polypeptide can include a covalently linked Schiff base modification, such as a fatty aldehyde, a malondialdehyde, and the like.

### Enzymes

An enzymatic cleavage product of a protein component of an arteriosclerotic plaque can be a cleavage product of an acid protease, a serine protease, a cysteine protease, an aspartic acid protease, a matrix metalloprotease (MMP), and the like. The enzymes are produced in vivo by a cell (e.g., an inflammatory cell) in the vasculature.

Proteolytic cleavage enzymes that may be present in the artery wall, and that can give rise to an enzymatic cleavage product of a protein component of an arteriosclerotic plaque, include, but are not limited to, acid proteases, serine proteases (e.g., elastase-like serine proteases; chymotrypsin-like serine proteases; cysteine proteases; aspartic acid proteases; MMPs; an ADAMTS (A Disintegrin And Metalloproteinase with Thrombospondin Motifs) protease (e.g., any one of ADAMTS-1 through ADAMTS-20); and the like. Proteolytic cleavage enzymes that may be present in the artery wall, and that can give rise to an enzymatic cleavage product of a protein component of an arteriosclerotic plaque, include, but are not limited to, cathepsins (e.g., cathepsin K, cathepsin S, cathepsin L, cathepsin B, cathepsin D, cathepsin H, and cystatin C); chymase; tryptase; macrophage metalloproteases; aggrecanases; protease-3; granzymes (e.g., granzyme A, granzyme B, granzyme H, granzyme K, etc.); and the like.

In some instances, an enzymatic cleavage product of a protein component of an arteriosclerotic plaque is a cleavage product of a matrix metalloproteinase (MMP), where MMPs include, e.g., secreted MMPs such as MMP1 (interstitial collagenase); MMP2 (72-kDa gelatinase, or gelatinase-A); MMP3 (stromelysin-1); MMP7 (matrilysin); MMP8 (neutrophil collagenase); MMP9 (92-kDa gelatinase or gelatinase-B); MMP10 (stromelysin-2); MMP11 (stromelysin-3); MMP12 (macrophage metalloprotease); MMP13 (collagenase-3); and MMP16. An enzymatic cleavage product of a protein component of an arteriosclerotic plaque can also be a cleavage product of a cathepsin. In some instances, cleavage products of a cathepsin are specifically excluded.

Enzymes that produce an enzymatic cleavage product of a protein component of an arteriosclerotic plaque can be produced by (e.g., secreted by) inflammatory cells, e.g., macrophages, neutrophils, monocytes, or transformed smooth muscle cells. Thus, in some embodiments, a subject method generally involves detecting in a biological sample from the individual an enzymatic cleavage product (e.g., an in vivo-generated enzymatic cleavage product) of a protein component of an arteriosclerotic plaque, where the enzymatic cleavage product is a product of cleavage by an enzyme produced by a cell (e.g., an inflammatory cell) in the vasculature.

An enzymatic cleavage product of a protein component of an arteriosclerotic plaque can have a signature structure characteristic of cleavage by an enzyme(s) produced by an inflammatory cell in the vasculature.

### Enzymatic Cleavage Products

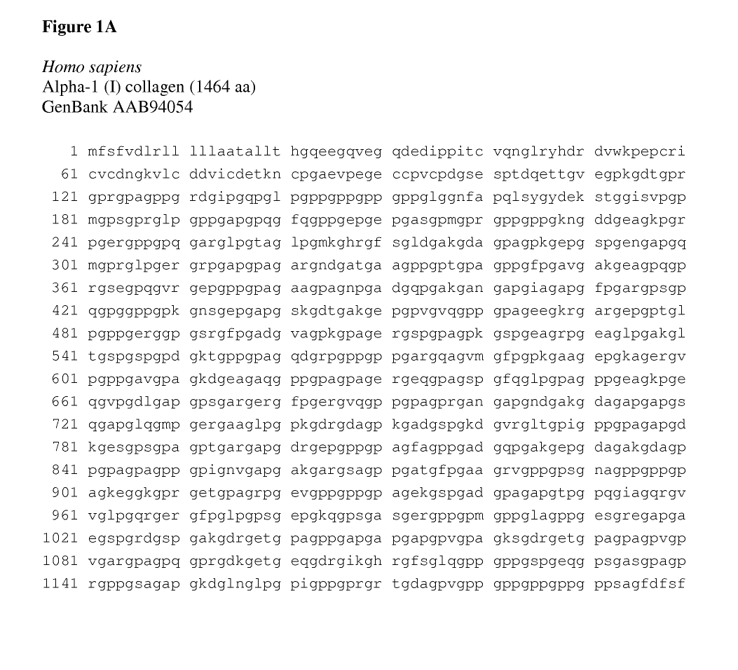

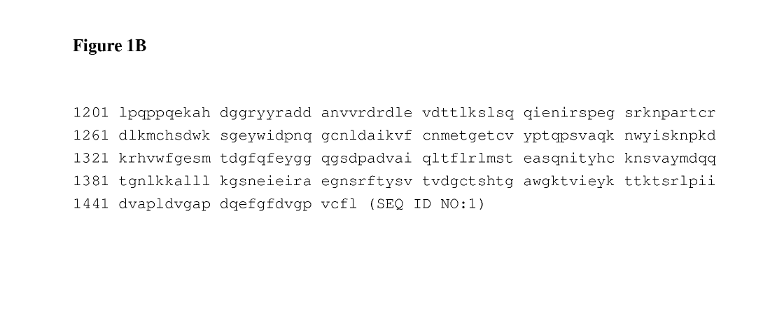

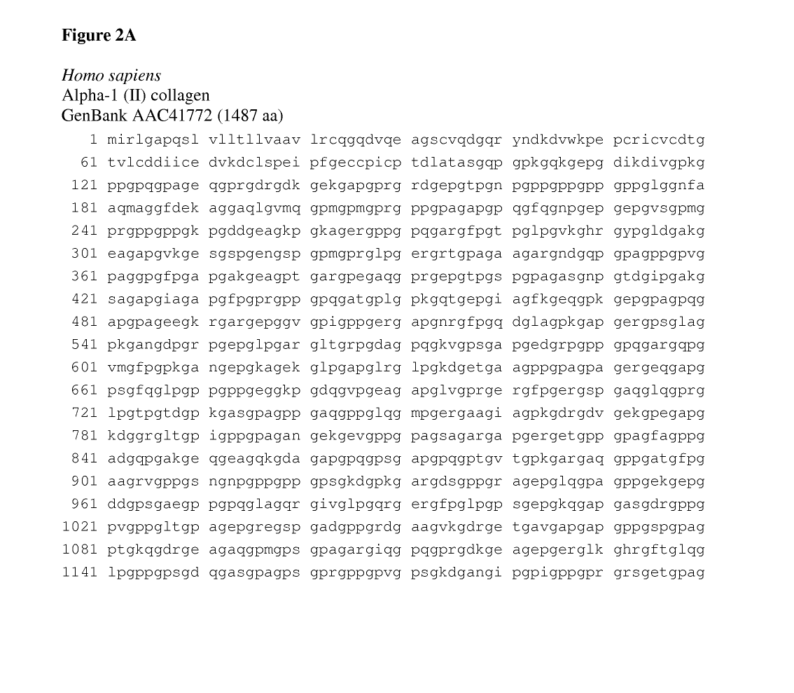

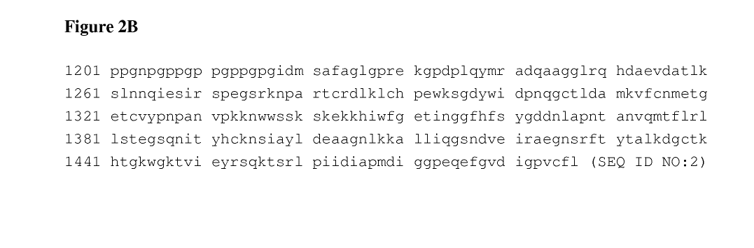

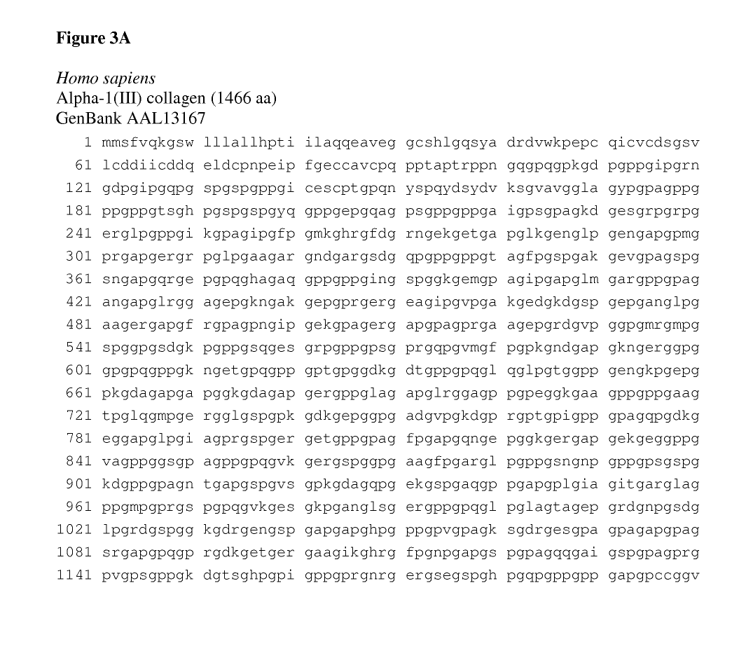

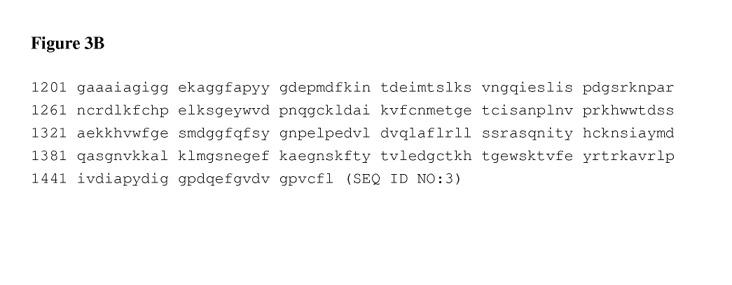

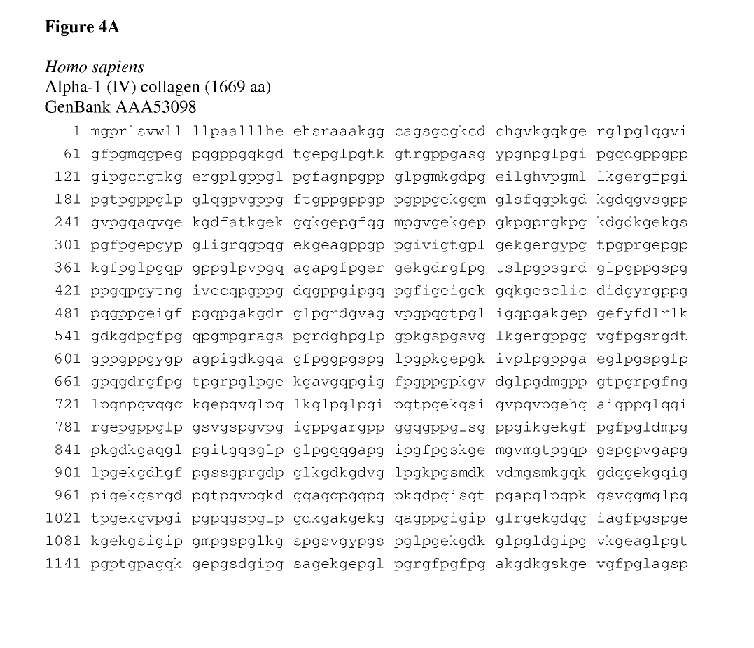

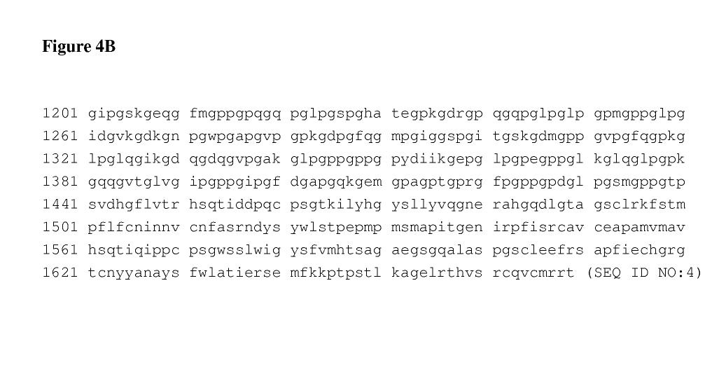

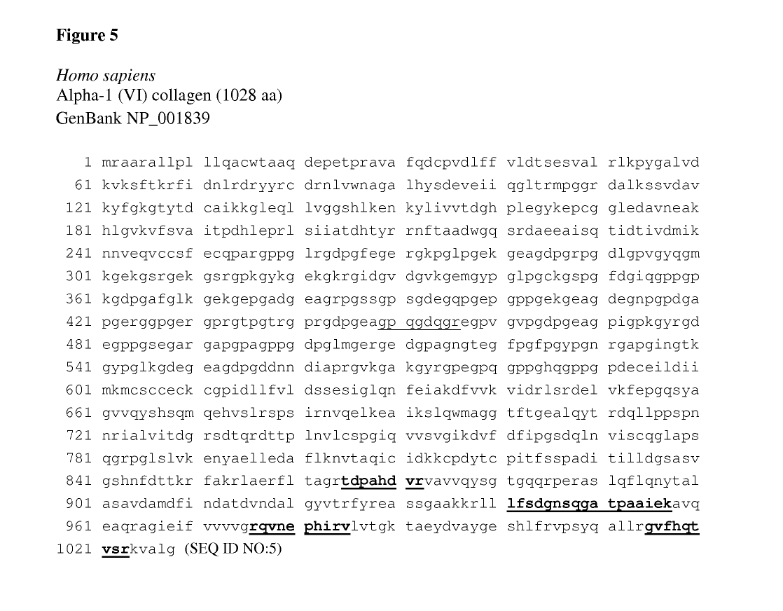

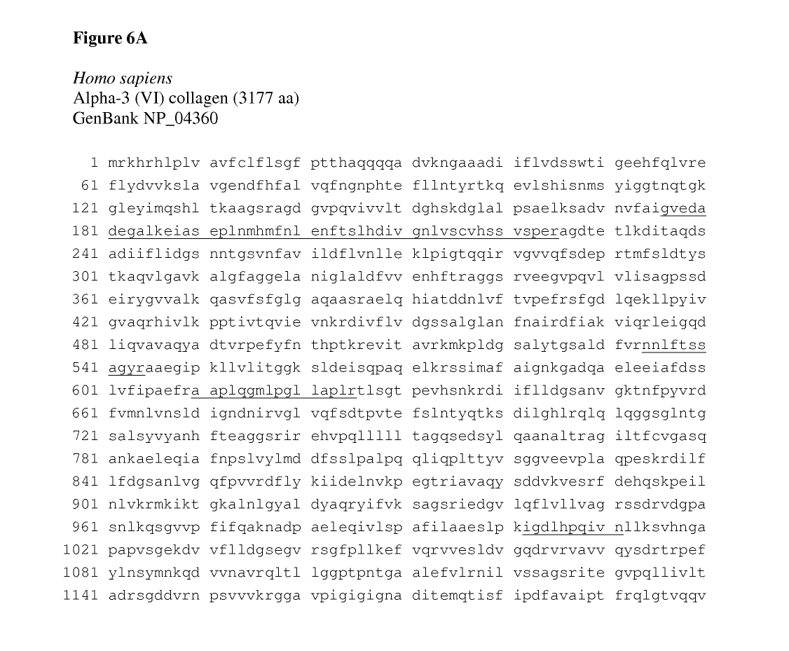

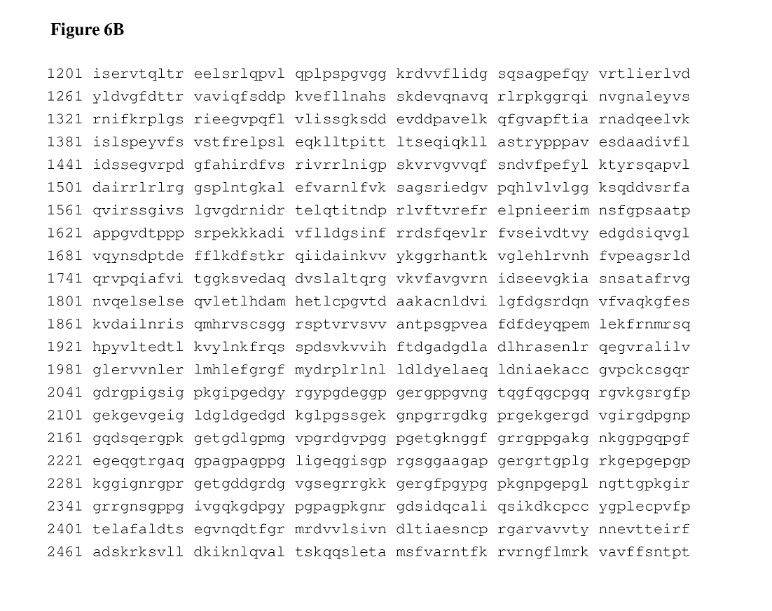

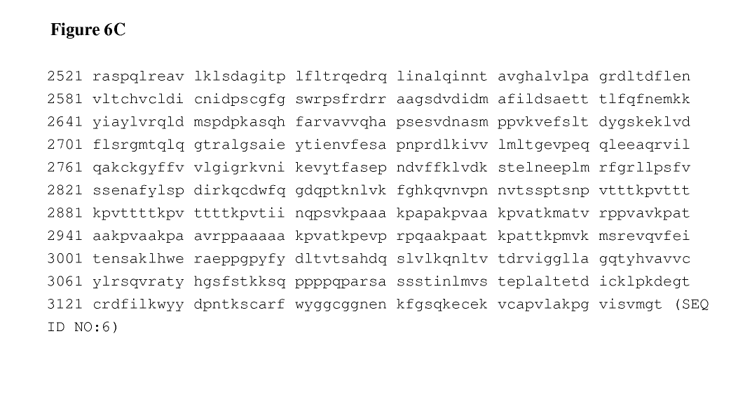

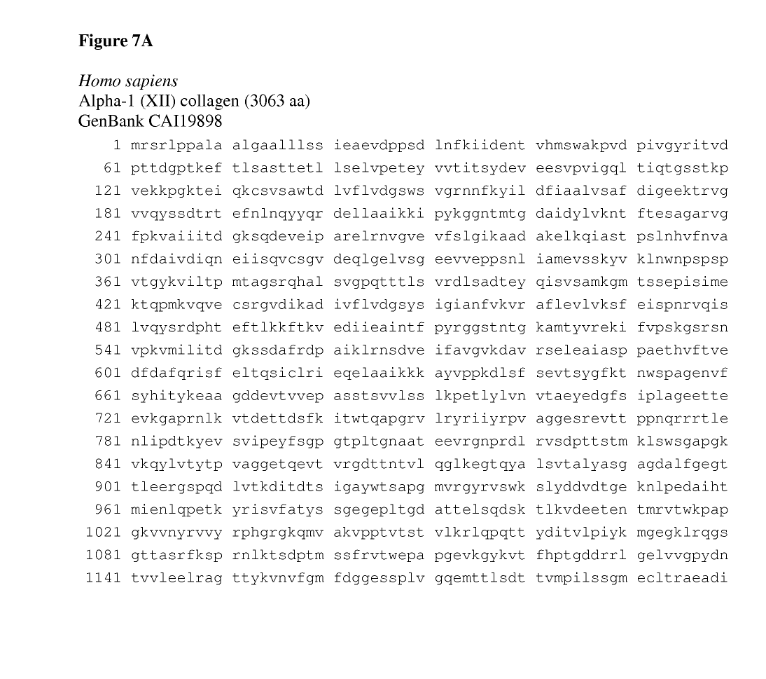

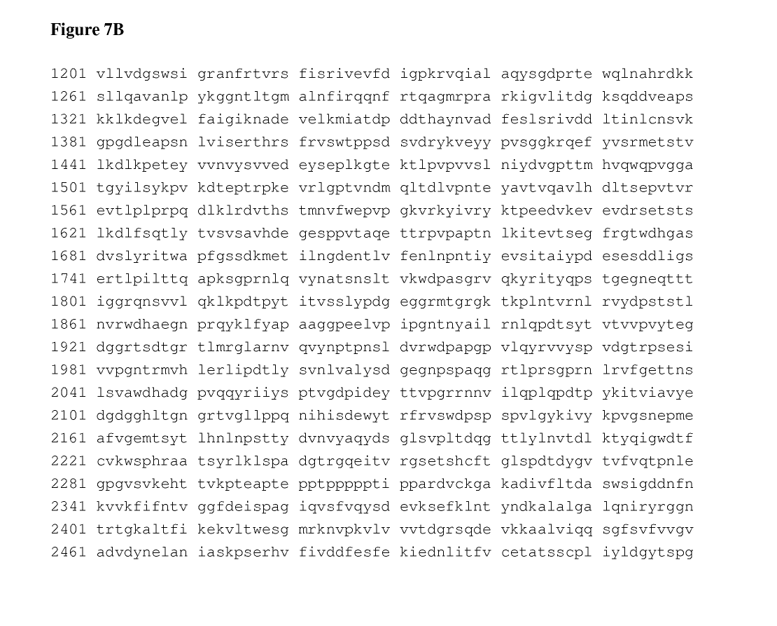

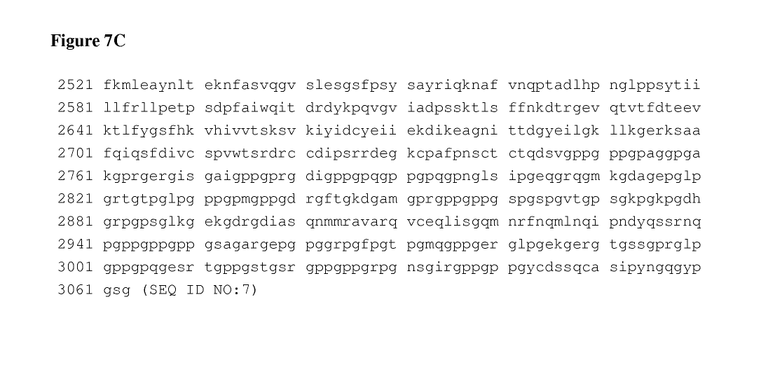

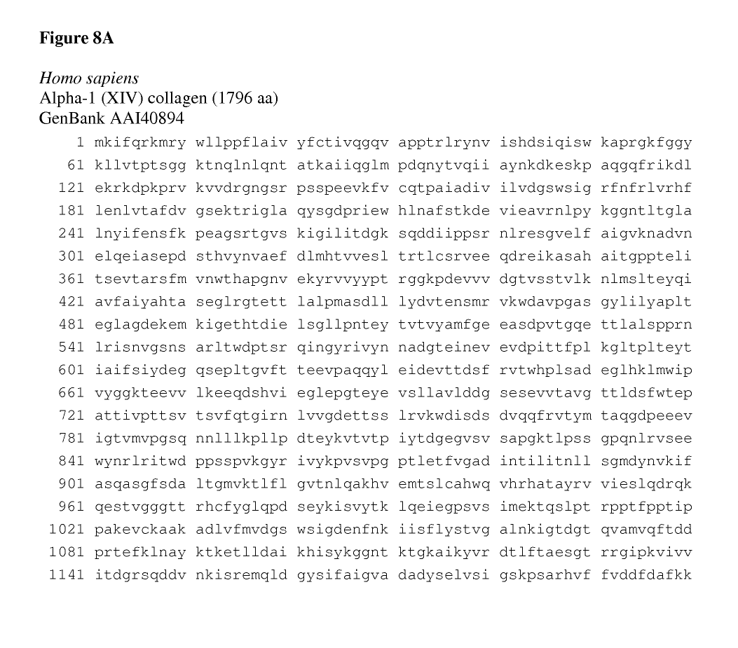

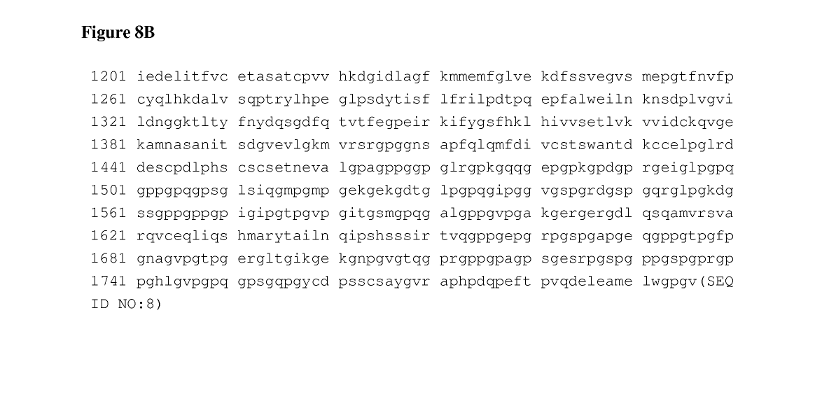

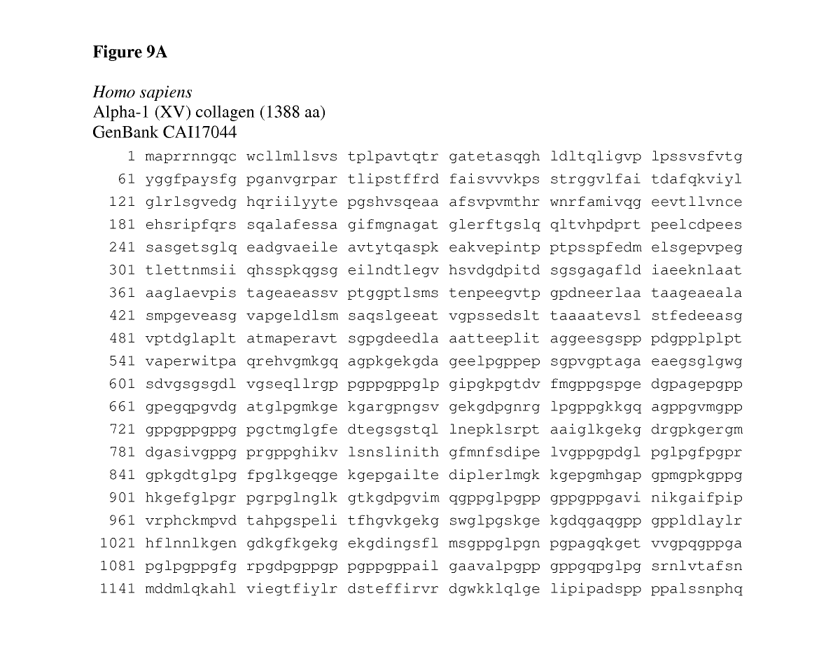

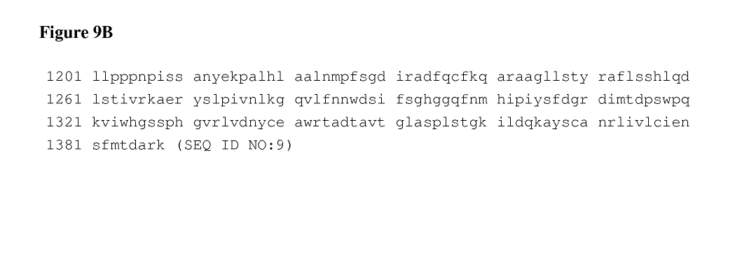

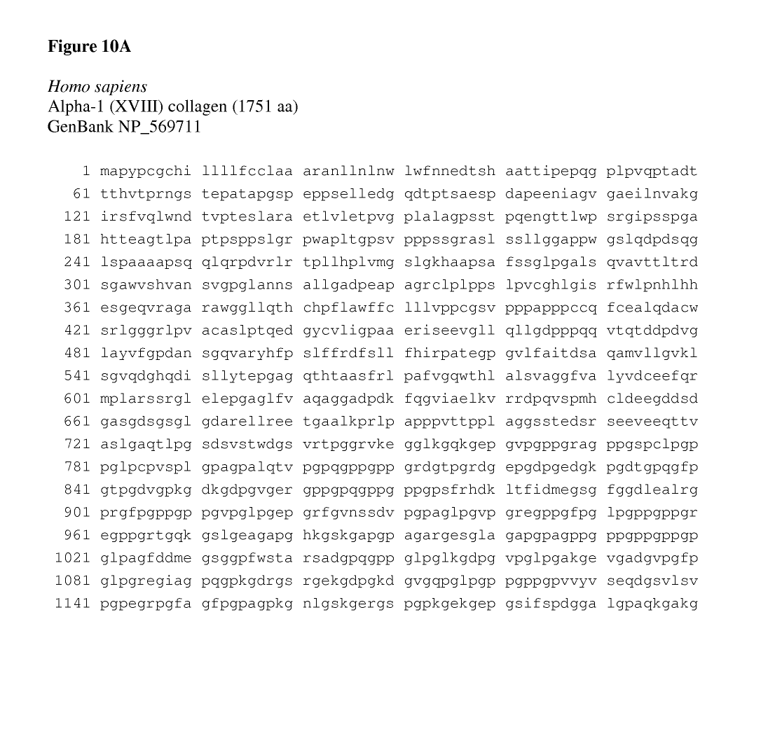

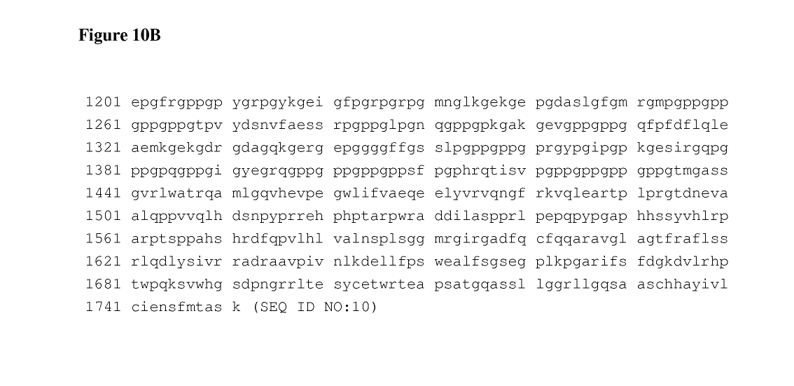

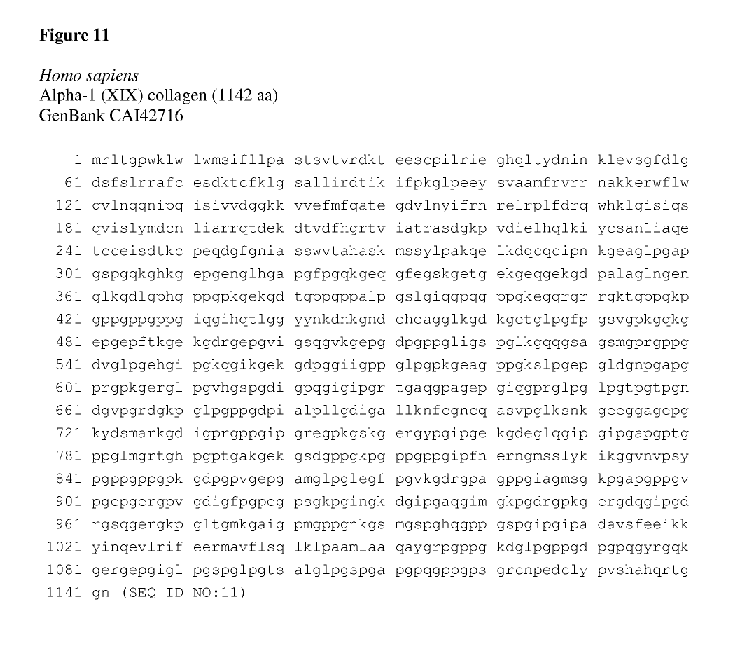

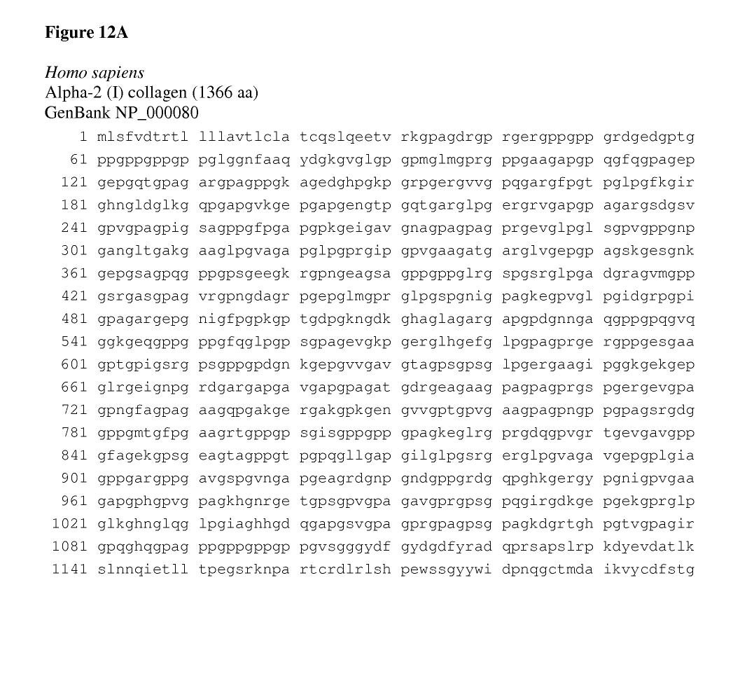

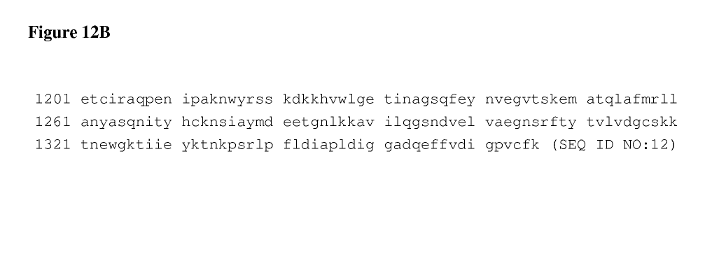

Enzymatic cleavage products of a protein component of an arteriosclerotic plaque include enzymatic cleavage products (enzymatic cleavage products generated by an enzyme produced by an inflammatory cell in the vasculature) of vascular collagen (where vascular collagen encompasses, e.g., collagen alpha-1 (I) chain; collagen alpha-1 (II) chain; collagen alpha-1 (III) chain; collagen alpha-1 (IV) chain; collagen alpha-1 (VI) chain; collagen alpha-1 (XII); collagen alpha-1 (XIV) chain; collagen alpha-1 (XV) chain; collagen alpha-1 (XVIII); collagen alpha-1 (XIX); collagen alpha-2 (I) chain; collagen alpha-3 (VI); collagen alpha-2 (IV); and collagen alpha-5 (IV)); fibrillin-1; fibronectin; vitronectin; metalloproteinase inhibitor 1; dermatopontin; galectin-1; prolargin; tenascin; and tenascin-X. Enzymatic cleavage products of a protein component of an arteriosclerotic plaque can also include enzymatic cleavage products (enzymatic cleavage products generated by an enzyme produced by an inflammatory cell in the vasculature) of collagen type III, elastin, decorin, biglycan, versican, apolipoprotein E, C-reactive protein, or lumican. In some embodiments, enzymatic cleavage products of one or more of collagen type III, elastin, decorin, biglycan, versican, apolipoprotein E, C-reactive protein, and lumican are specifically excluded. Amino acid sequences of such proteins are known in the art. Exemplary sequences are provided in FIGS. 1-24.

Enzymatic cleavage products to be detected according to a method of the present disclosure can include cleavage products of all, or a subset of, the above-listed proteins. For example, enzymatic cleavage products of a protein component of an arteriosclerotic plaque can include enzymatic cleavage products of all 23, or a subset of 2, 3, 4, 5, 6, 7, 8, 9, 10, 11, 12, 13, 14, 15, 16, 17, 18 19, 20, 21, or 22, of the following set of proteins: 1) fibrillin (fibrillin-1); 2) vitronectin; 3) fibronectin; 4) tenascin; 5) prolargin; 6) dermatopontin; 7) collagen alpha-1 (I) chain; 8) collagen alpha-1 (II) chain; 9) collagen alpha-1 (III) chain; 10) collagen alpha-1 (IV) chain; 11) collagen alpha-1 (VI) chain; 12) collagen alpha-1 (XII); 13) collagen alpha-1 (XIV) chain; 14) collagen alpha-1 (XV) chain; 15) collagen alpha-1 (XVIII); 16) collagen alpha-1 (XIX); 17) collagen alpha-2 (I) chain; 18) collagen alpha-3 (VI); 19) collagen alpha-2 (IV); 20) collagen alpha-5 (IV); 21) metalloproteinase inhibitor 1; 22) galectin-1; and 23) tenascin-X. As an example, enzymatic cleavage products of a protein component of an arteriosclerotic plaque can include enzymatic cleavage products of: collagen alpha-1 (I) chain; collagen alpha-1 (II) chain; collagen alpha-1 (IV) chain; collagen alpha-1 (VI) chain; collagen alpha-1 (XII); collagen alpha-1 (XIV) chain; collagen alpha-1 (XV) chain; collagen alpha-1 (XVIII); collagen alpha-1 (XIX); collagen alpha-2 (I) chain; collagen alpha-3 (VI); collagen alpha-2 (IV); collagen alpha-5 (IV); fibrillin-1; fibronectin; vitronectin; metalloproteinase inhibitor 1; dermatopontin; galectin-1; prolargin; tenascin; and tenascin-X.

Enzymatic cleavage products of one or more of the above-listed proteins can in certain instances be specifically excluded. For example, in some instances, an enzymatic cleavage product of collagen type III is specifically excluded. In some instances, an enzymatic cleavage product of lumican is specifically excluded. In some instances, an enzymatic cleavage product of elastin is specifically excluded. In some instances, an enzymatic cleavage product of one or more of versican, perlecan, decorin, and biglycan is specifically excluded. In some instances, an enzymatic cleavage product of versican, perlecan, decorin, and biglycan is specifically excluded. In some instances, an enzymatic cleavage product of C-reactive protein (CRP) is specifically excluded. In some instances, an enzymatic cleavage product of apolipoprotein-E is specifically excluded.

Enzymatic cleavage products of a protein component of an arteriosclerotic plaque can have a size in a range of from about 0.5 kDa to about 50 kDa, e.g., from about 0.5 kDa to about 1 kDa, from about 1 kDa to about 1.5 kDa, from about 1.5 kDa to about 2 kDa, from about 2 kDa to about 5 kDa, from about 5 kDa to about 7.5 kDa, from about 7.5 kDa to about 10 kDa, from about 10 kDa to about 15 kDa, from about 15 kDa to about 20 kDa, from about 20 kDa to about 25 kDa, from about 25 kDa to about 30 kDa, from about 30 kDa to about 35 kDa, from about 35 kDa to about 40 kDa, from about 40 kDa to about 45 kDa, or from about 45 kDa to about 50 kDa.

An enzymatic cleavage product of a protein component of an arteriosclerotic plaque can have a length of from about 5 amino acids to about 500 amino acids, e.g., from about 5 amino acids (aa) to about 10 aa, from about 10 aa to about 15 aa, from about 15 aa to about 20 aa, from about 20 aa to about 25 aa, from about 25 aa to about 30 aa, from about 30 aa to about 35 aa, from about 35 aa to about 40 aa, from about 40 aa to about 45 aa, from about 45 aa to about 50 aa, from about 50 aa to about 75 aa, from about 75 aa to about 100 aa, from about 100 aa to about 150 aa, from about 150 aa to about 200 aa, from about 200 aa to about 250 aa, from about 250 aa to about 300 aa, from about 300 aa to about 350 aa, from about 350 aa to about 400 aa, from about 400 aa to about 450 aa, or from about 450 aa to about 500 aa.

### Collagens

Collagens are extracellular matrix proteins and have a triple-helical domain as their common structural element. Each collagen molecule includes three polypeptides referred to as alpha chains. For example, the basic structural unit of collagen VI is a heterotrimer of the alpha-1(VI), alpha-2(VI), and alpha-3(VI) chains.

Vascular collagens that are structural components of arteriosclerotic plaques include collagen alpha-1 (I) chain; collagen alpha-1 (II) chain; collagen alpha-1 (III) chain; collagen alpha-1 (IV) chain; collagen alpha-1 (VI) chain; collagen alpha-1 (XII); collagen alpha-1 (XIV) chain; collagen alpha-1 (XV) chain; collagen alpha-1 (XVIII); collagen alpha-1 (XIX); collagen alpha-2 (I) chain; and collagen alpha-3 (VI). Vascular collagen is present in the vasculature.

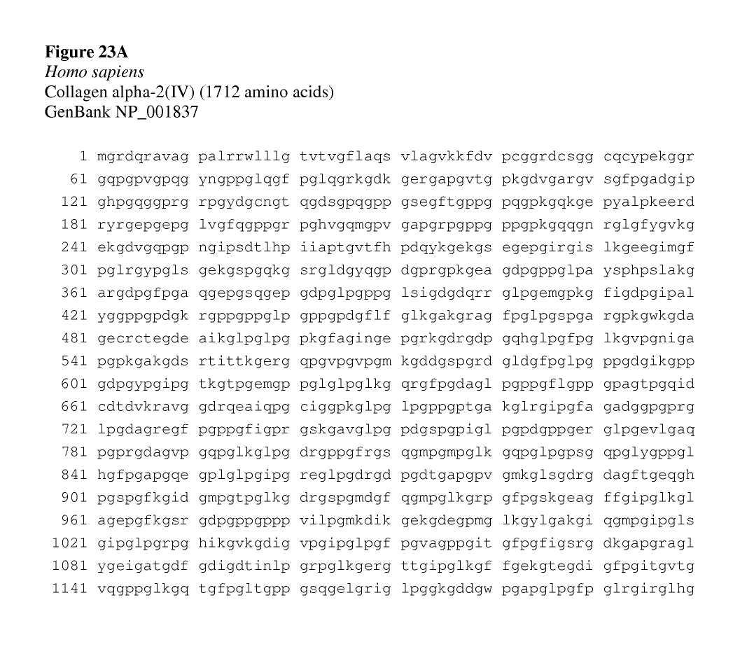

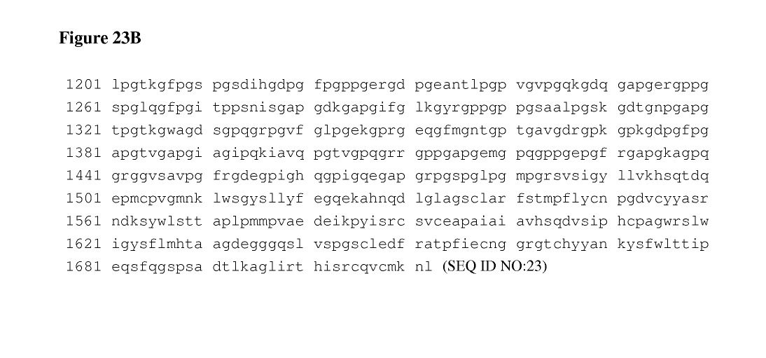

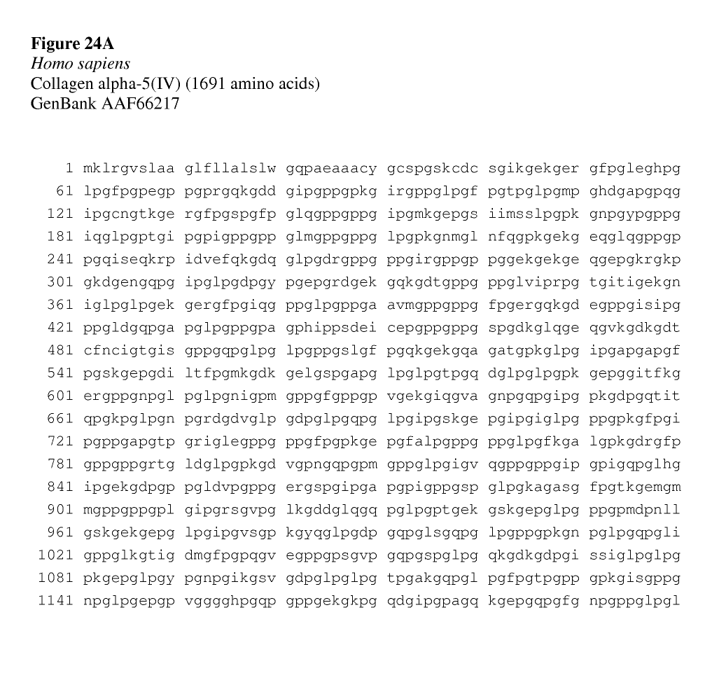

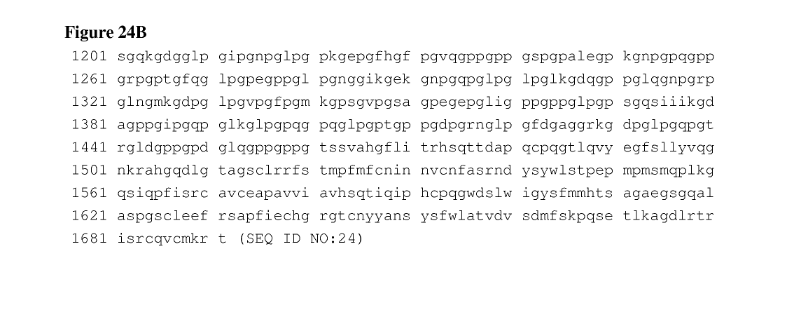

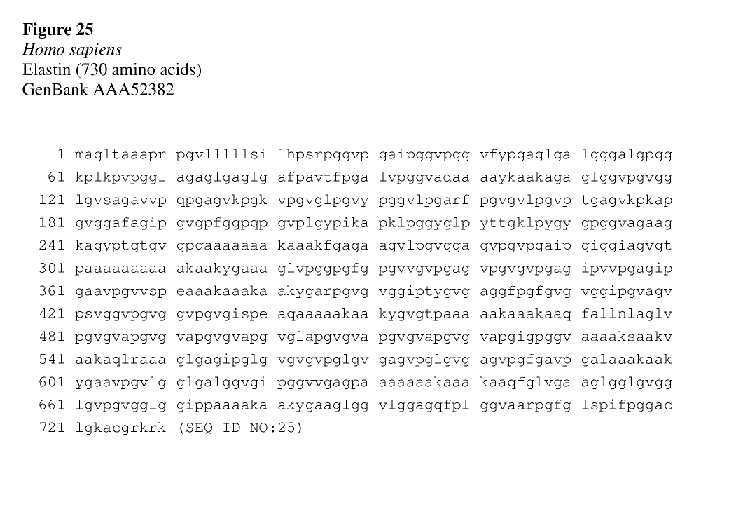

Exemplary amino acid sequences of collagen alpha-1 (I) chain; collagen alpha-1 (II) chain; collagen alpha-1 (III) chain; collagen alpha-1 (IV) chain; collagen alpha-1 (VI) chain; collagen alpha-1 (XII); collagen alpha-1 (XIV) chain; collagen alpha-1 (XV) chain; collagen alpha-1 (XVIII); collagen alpha-1 (XIX); collagen alpha-2 (I) chain; and collagen alpha-3 (VI) are provided in FIGS. 1-12. Exemplary amino acid sequences of collagen alpha-2 (IV) and collagen alpha-5 (IV) are provided in FIGS. 23 and 24.

An enzymatic cleavage product of a vascular collagen component of an arteriosclerotic plaque can be an enzymatic cleavage product of a collagen polypeptide having at least about 98%, at least about 99%, or 100%, amino acid sequence identity with an amino acid sequence of a collagen polypeptide depicted in one of FIGS. 1-12, 23, and 24. An enzymatic cleavage product of a vascular collagen component of an arteriosclerotic plaque can be an enzymatic cleavage product of a naturally-occurring variant (polymorphism) of a collagen polypeptide depicted in one of FIGS. 1-12, 23, and 24.

### Collagen Alpha-1(I) Proteolytic Fragments

Non-limiting examples of enzymatic cleavage products (enzymatic cleavage products generated by an enzyme produced by a cell (e.g., an inflammatory cell) in the vasculature) of a Collagen alpha-1(I) chain, listed in sequence order, include:

(SEQ ID NO: 26)
TGGISVPGPMGPSGPR;
(SEQ ID NO: 27)
GLPGPPGAPGPQG;
(SEQ ID NO: 28)
GLPGPPGAPGPQGF;
(SEQ ID NO: 29)
PGEPGEPGASGPMGPRGPPGPPGK;
(SEQ ID NO: 30)
GASGPMGPRGPPGPPGK;
(SEQ ID NO: 31)
KPGRPGERGPPGPQGAR;
(SEQ ID NO: 32)
GPPGPQGARGLPGTAGLPGM;
(SEQ ID NO: 33)
AGPQGPR;
(SEQ ID NO: 34)
GAPGIAGAPGFPGAR;
(SEQ ID NO: 35)
PGIAGAPGFPGARGPSGPQGPGGPPGPK;
(SEQ ID NO: 36)
IAGAPGFPGARGPSGPQGPGGPPGPK;
(SEQ ID NO: 37)
GFPGARGPSGPQGPGGPPGPK;
(SEQ ID NO: 38)
GPSGPQGPGG;
(SEQ ID NO: 39)
GDTGAKGEP;
(SEQ ID NO: 40)
VQGPPGPAGEEGK;
(SEQ ID NO: 41)
GEPGPTGLPGPPG;
(SEQ ID NO: 42)
GEPGPTGLPGPPGERGGPGS;
(SEQ ID NO: 43)
TGLPGPPGER;
(SEQ ID NO: 44)
LPGPPGER;
(SEQ ID NO: 45)
AGPKGPAGER;
(SEQ ID NO: 46)
GSPGPAGPKGSPGEAGRPGEAG;
(SEQ ID NO: 47)
PGEAGRPGEAGLPGAKGLTGSPGSPGPDGK;
(SEQ ID NO: 48)
LTGSPGSPGPDGK;
(SEQ ID NO: 49)
TGPPGPAGQDGRPGPPGPPGARG;
(SEQ ID NO: 50)
PGAVGPAGKDGEAGAQGPPGPAGPAGER;
(SEQ ID NO: 51)
GEAGAQGPPGPAGPAGER;
(SEQ ID NO: 52)
EAGAQGPPGPAGPAGER;
(SEQ ID NO: 53)
VQGPPGPAGPR;
(SEQ ID NO: 54)
QGPPGPAGPR*;
(SEQ ID NO: 55)
GPPGPAGPR;
(SEQ ID NO: 56)
ANGAPGNDGAKGDAGAPGAPGSQGAPGLQGMPGER;
(SEQ ID NO: 57)
LQGMPGER*;
(SEQ ID NO: 58)
LTGPIGPPGPAGAPGDK;
(SEQ ID NO: 59)
IGPPGPAGAPGDK;
(SEQ ID NO: 60)
KGESGPSGPAGPTGAR;
(SEQ ID NO: 61)
PGDRGEPGPPGPAGFAGPPGADGQPGAK;
(SEQ ID NO: 62)
GEPGPPGPAGF;
(SEQ ID NO: 63)
FAGPPGADGQPGAK*;
(SEQ ID NO: 64)
AGPPGADGQPGAK*;
(SEQ ID NO: 65)
RVGPPGPSGNAGPPGPPGPAGK;
(SEQ ID NO: 66)
VGPPGPSGNAGPPGPPGPAGKEGG;
(SEQ ID NO: 67)
EVGPPGPPGPAGEKGSPGADGPAGAPGTPGPQGIAGQR;
(SEQ ID NO: 68)
PGPPGPAGEKGSPGADGPAGAPGTPGPQGIAGQR;
(SEQ ID NO: 69)
GSPGADGPAGAPGTPGPQG;
(SEQ ID NO: 70)
GPAGAPGTPGPQGIAGQR;
(SEQ ID NO: 71)
VVGLPGQR;
(SEQ ID NO: 72)
LAGPPGESGR;
(SEQ ID NO: 73)
ETGPAGPPGAPGAPGAPGPVGPAGKSGDR;
(SEQ ID NO: 74)
RGETGPAGPAGPVGPVGAR;
(SEQ ID NO: 75)
PAGPVGPVGAR;
(SEQ ID NO: 76)
PVGPVGAR;
(SEQ ID NO: 77)
SPGEQGPSGASGPAGPR;
(SEQ ID NO: 78)
PGEQGPSGASGPAGPR;
(SEQ ID NO: 79)
GPSGASGPAGPR;
(SEQ ID NO: 80)
ASGPAGPR;
(SEQ ID NO: 81)
GPPGSAGAPGKD;
(SEQ ID NO: 82)
PPGSAGAPGKDGLNGLPGPIGPPGPR;
and
(SEQ ID NO: 83)
LPQPPQEK*,

and naturally-occurring variants of any of the foregoing.

### Collagen Alpha-2(I) Proteolytic Fragments

Non-limiting examples of enzymatic cleavage products (enzymatic cleavage products generated by an enzyme produced by a cell (e.g., an inflammatory cell) in the vasculature) of a Collagen alpha-2(I) chain, listed in sequence order, include:

(SEQ ID NO: 84)
GLMGPRGPPGAAGAPGPQGFQGPAGEPGEPGQTGPAGAR;
(SEQ ID NO: 85)
FQGPAGEPGEPGQTGPAGAR;
(SEQ ID NO: 86)
QGPAGEPGEPGQTGPAGAR;
(SEQ ID NO: 87)
EDGHPGKPGRPGERGVVGPQGAR;
(SEQ ID NO: 88)
PAGARGSDGSVGPVGPAGPIGSAGPPGFPGAPGPK;
(SEQ ID NO: 89)
DGSVGPVGPAGPIGSAGPPGFPGAPGPK;
(SEQ ID NO: 90)
PGAPGPKGEIGAVGNAGPAGPAGPR;
(SEQ ID NO: 91)
GPAGPAGPR;
(SEQ ID NO: 92)
PAGPAGPR;
(SEQ ID NO: 93)
RGEVGLPGLSGPVGPPGNPGANGLTGAK;
(SEQ ID NO: 94)
GAPGLPGPR;
(SEQ ID NO: 95)
PNGEAGSAGPPGPPGLR;
(SEQ ID NO: 96)
GPRGLPGSPGNIGPAGK;
(SEQ ID NO: 97)
GRPGPIGPAGAR;
(SEQ ID NO: 98)
GPSGPPGPDG;
(SEQ ID NO: 99)
GPSGPPGPDGNKGEPGVVGAVGTAGPS;
(SEQ ID NO: 100)
GPSGLPGER;
(SEQ ID NO: 101)
GAVGAPGPAGATGDRGEAGAAGPAGPAGPR;
(SEQ ID NO: 102)
VGAPGPAGATGDRGEAGAAGPAGPAGPR;
(SEQ ID NO: 103)
PGPAGATGDRGEAGAAGPAGPAGPR;
(SEQ ID NO: 104)
NGVVGPTGPVGAAGPAGPNGPPGPAGSR;
(SEQ ID NO: 105)
GPPGPAGSR;
(SEQ ID NO: 106)
PGPAGSRGDGGPPGMTGFPGAAGR;
(SEQ ID NO: 107)
GDGGPPGMTGFPGAAGRTGPPGPSGISGPPGPPGPA;
(SEQ ID NO: 108)
ISGPPGPPGPAGK;
(SEQ ID NO: 109)
GPSGEAGTAGPPGTPGPQGL;
(SEQ ID NO: 110)
PGILGLPGSR;
(SEQ ID NO: 111)
IAGPPGAR;
(SEQ ID NO: 112)
PGNIGPVGAAGAPGPHGPVGPAGKHGNR;
(SEQ ID NO: 113)
VGPAGAVGPR;
(SEQ ID NO: 114)
QGAPGSVGPAGPR;
(SEQ ID NO: 115)
GPAGPSGPAGKD;
and
(SEQ ID NO: 116)
GTVGPAGIR,

and naturally-occurring variants of any of the foregoing.

### Collagen Alpha-1(III) Proteolytic Fragments

Non-limiting examples of enzymatic cleavage products (enzymatic cleavage products generated by an enzyme produced by a cell (e.g., an inflammatory cell) in the vasculature) of a Collagen alpha-1(III) chain, listed in sequence order, include:

(SEQ ID NO: 117)
GPQGPKGDPGPPGIPGR;
(SEQ ID NO: 118)
PGTSGHPGSPGSPGYQGPPGEPGQAGPSGPPGPPGAIGPSGPAGK;
(SEQ ID NO: 119)
GLPGPPGIKGPAG;
(SEQ ID NO: 120)
GEVGPAGSPGSNGAPGQRGEPGPQGHAG;
(SEQ ID NO: 121)
GEPGPQGHAGAQGPPGPPGINGSPGGKGEMGPAGIPG;
(SEQ ID NO: 122)
GEMGPAGIPGAPGLMGARGPPGPAG;
(SEQ ID NO: 123)
GIPGAPGLMGAR;
(SEQ ID NO: 124)
GAPGLMGARGPPGPAGANGAPGLR;
(SEQ ID NO: 125)
PAGERGAPGPAGPR;
(SEQ ID NO: 126)
GAPGPAGPRGAAGEP;
(SEQ ID NO: 127)
GEPGRDGVPGGPGMR;
(SEQ ID NO: 128)
DGKPGPPGSQGESGRPGPPGPSGPR;
(SEQ ID NO: 129)
GKPGPPGSQGESGRPGPPGPSGPR;
(SEQ ID NO: 130)
GRPGPPGPSGPR;
(SEQ ID NO: 131)
GPPGPSGPR;
(SEQ ID NO: 132)
QGPPGKNGETGPQGPPGPTGPGGDK;
(SEQ ID NO: 133)
GDAGAPGERGP;
(SEQ ID NO: 134)
LQGMPGER;
(SEQ ID NO: 135)
GEGGPPGVAGPPGGSGPAGPPGPQGV;
(SEQ ID NO: 136)
GSNGNPGPPGPSGSPGKDGPPGPAGNTGAPGSPGVSGPK;
(SEQ ID NO: 137)
NGNPGPPGPSGSPGK;
(SEQ ID NO: 138)
GSPGAQGPP;
(SEQ ID NO: 139)
GNPGSDGLPGR;
(SEQ ID NO: 140)
ENGSPGAPGAPGHPGPPGPVGPAGK;
(SEQ ID NO: 141)
PGAPGAPGHPGPPGPVGPAGK;
(SEQ ID NO: 142)
RGESGPAGPAGAPGPAGSR;
(SEQ ID NO: 143)
GESGPAGPAGAP;
(SEQ ID NO: 144)
PGAPGSPGPAGQQGAIGSPGPAGPR;
(SEQ ID NO: 145)
GQQGAIGSPGPAGPRGPVGPSGPPGK;
(SEQ ID NO: 146)
QGAIGSPGPAGPR;
and
(SEQ ID NO: 147)
GSEGSPGHPGQPGPPGPPGAPGPCCGGVGAAAIAGIGGEKAGGFAPYYG,

and naturally-occurring variants of any of the foregoing.

### Collagen Alpha-1(II) Proteolytic Fragments

Non-limiting examples of enzymatic cleavage products (enzymatic cleavage products generated by an enzyme produced by a cell (e.g., an inflammatory cell) in the vasculature) of a Collagen alpha-1(II) chain, listed in sequence order, include:

(SEQ ID NO: 148)
GPQGFQGNPGEPGEPGVSGPMGPRGPPGPPGKPGDDGEAGKPGK;
(SEQ ID NO: 149)
GAAGARGNDGQPGPAGPPGPVGPAGGPGFPGAPGAK;
(SEQ ID NO: 150)
AAGARGNDGQPGPAGPPGPVGPAGGPGFPGAPGAK;
(SEQ ID NO: 151)
PGAKGSAGAPGIAGAPGFPGPR;
(SEQ ID NO: 152)
GPRGPPGPQGATGPLGPK;
(SEQ ID NO: 153)
DGLAGPK;
(SEQ ID NO: 154)
PQGKVGPSGAPGEDGRPGPPGPQGAR;
(SEQ ID NO: 155)
GFPGPKGANGEPGK;
(SEQ ID NO: 156)
GLPGPPGPPGEGGKPGDQGVPGEAGAPGLVGPR;
(SEQ ID NO: 157)
GPPGEGGKPGDQGVPGEAGAPGLVGPR;
(SEQ ID NO: 158)
LQGMPGER;
(SEQ ID NO: 159)
GRGLTGPIGPPGPAGANGEK;
(SEQ ID NO: 160)
GLTGPIGPPGPAGANGEKGEVGPP;
(SEQ ID NO: 161)
GLTGPIGPPGPAGANGEKGEVGPPGPAGSAG;
(SEQ ID NO: 162)
LTGPIGPPGPAGANGEKGEVGPPGPAGSAGAR;
(SEQ ID NO: 163)
TGPIGPPGPAGANGEKGEVGPPGPAGSAGAR;
(SEQ ID NO: 164)
FAGPPGADGQPGAK*;
(SEQ ID NO: 165)
AGPPGADGQPGAK*;
(SEQ ID NO: 166)
SGPPGRAGEPGLQGPAGPPGEK;
(SEQ ID NO: 167)
GPPGRAGEPGLQGPAGPPGEK;
(SEQ ID NO: 168)
PPGLTGPAGEPGREGSPGADGPPGR;
and
(SEQ ID NO: 169)
PGPGIDMSAFAGLGPREK,

and naturally-occurring variants of any of the foregoing.

### Collagen Alpha-1(XIV) Proteolytic Fragments

Non-limiting examples of enzymatic cleavage products (enzymatic cleavage products generated by an enzyme produced by a cell (e.g., an inflammatory cell) in the vasculature) of a Collagen alpha-1(XIV) chain, listed in sequence order, include:

(SEQ ID NO: 170)
IEWHLNAF;
(SEQ ID NO: 171)
AITGPPTELITSEVTAR;
(SEQ ID NO: 172)
AIYAHTASEGLR;
(SEQ ID NO: 173)
LYDVTENSMR;
(SEQ ID NO: 174)
YLILYAPLTEGLAGDEKEMK;
(SEQ ID NO: 175)
YAPLTEGLAGDEK;
(SEQ ID NO: 176)
HVEMTSLCAH;
(SEQ ID NO: 177)
SIQGMPGMPGEKGEK;
(SEQ ID NO: 178)
QVCEQLIQSH;
and
(SEQ ID NO: 179)
EPGRPGSPGAPGEQGPPGTPGFPGNAGVPGTPGER,

and naturally-occurring variants of any of the foregoing.

### Collagen Alpha-1(XII) Proteolytic Fragments

Non-limiting examples of enzymatic cleavage products (enzymatic cleavage products generated by an enzyme produced by a cell (e.g., an inflammatory cell) in the vasculature) of a Collagen alpha-1(XII) chain, listed in sequence order, include:

(SEQ ID NO: 180)
GGSTNTGKAMTYVRE;
(SEQ ID NO: 181)
PKVMILITDGK;
(SEQ ID NO: 182)
PDDTHAYNVADFESLSR;
(SEQ ID NO: 183)
SVVEDEYSEPLK;
(SEQ ID NO: 184)
SETSTSLKD;
(SEQ ID NO: 185)
LKPDTPYTITVSSLYPDGEGGRMTG;
(SEQ ID NO: 186)
PGPAGGPGAK;
(SEQ ID NO: 187)
GRTGTPGLPGPPGPMGPPGDR;
(SEQ ID NO: 188)
TPGLPGPPGPMGPPGDRGFTGK;
(SEQ ID NO: 189)
GFPGTPGMQGPPGERGLPGEK;
(SEQ ID NO: 190)
QGPPGER;
and
(SEQ ID NO: 191)
PRGLPGPPGPQGESR,

and naturally-occurring variants of any of the foregoing.

### Collagen Alpha-1(XVIII) Proteolytic Fragments

Non-limiting examples of enzymatic cleavage products (enzymatic cleavage products generated by an enzyme produced by a cell (e.g., an inflammatory cell) in the vasculature) of a Collagen alpha-1(XVIII) chain, listed in sequence order, include:

(SEQ ID NO: 192)
PPSLGRPWAPLTGPSVPPPSSGR;
(SEQ ID NO: 193)
PGEDGKPGDTGPQGFPGTPGDVGPKGDK;
(SEQ ID NO: 194)
PGLPGEPGR;
(SEQ ID NO: 195)
GREGPPGFPGLPGPPGPPGR;
(SEQ ID NO: 196)
QDGSVLSVPGPEGRPGFAGFPGPAGPKGNLGSK;
(SEQ ID NO: 197)
AESSRPGPPGLPGNQGPPGPK;
(SEQ ID NO: 198)
GPPGPKGAK;
(SEQ ID NO: 199)
PGPPGPPGTMGASSGVR;
(SEQ ID NO: 200)
RLPEPQPYPGAPHHSSYVHLRPARPTSPPAHSHR;
(SEQ ID NO: 201)
LPEPQPYPGAPHHSSY;
(SEQ ID NO: 202)
NSPLSGGMR;
and
(SEQ ID NO: 203)
PSLGRPWAPLTGPSVPPPSSER,

and naturally-occurring variants of any of the foregoing.

### Collagen Alpha-2(IV) Proteolytic Fragments

Non-limiting examples of enzymatic cleavage products (enzymatic cleavage products generated by an enzyme produced by a cell (e.g., an inflammatory cell) in the vasculature) of a Collagen alpha-2(IV) chain, listed in sequence order, include:

(SEQ ID NO: 204)
GARGVSGFPGADGIPGHPGQGGPR;
(SEQ ID NO: 205)
GGPKGLPGLPGPPGPTGAK;
(SEQ ID NO: 206)
GPPGLHGFPGAPGQEGPLGLPGIPGREGLPGDR;
(SEQ ID NO: 207)
APGRPGSPGLPGMPGR;
(SEQ ID NO: 208)
LYCNPGDVCYYASR;
and
(SEQ ID NO: 209)
LMHTAAGDEGGGQSLVSPGSCLEDFR,

and naturally-occurring variants of any of the foregoing.

### Collagen Alpha-1(IV) Proteolytic Fragments

Non-limiting examples of enzymatic cleavage products (enzymatic cleavage products generated by an enzyme produced by a cell (e.g., an inflammatory cell) in the vasculature) of a Collagen alpha-1(IV) chain, listed in sequence order, include:

and naturally-occurring variants of any of the foregoing.

(SEQ ID NO: 210)
EPGPPGLPGSVGSPGVPGIGPPGAR;
(SEQ ID NO: 211)
PGVPGIGPPGARGPPGGQGPPGLSGPPGIK;
(SEQ ID NO: 212)
PPGGQGPPGLSGPPGIKGEK;
(SEQ ID NO: 213)
DPGFQGMPGIGGSPGITGSK;
(SEQ ID NO: 214)
KGQQGVTGLVGIPGPPGIPGFDGAPGQK;
(SEQ ID NO: 215)
SLLYVQGNER;
(SEQ ID NO: 216)
LFCNINNVCNFASR;
(SEQ ID NO: 217)
VMHTSAGAEGSGQALASPGSCLEEFR;
(SEQ ID NO: 218)
RSAPFIECHGR;
(SEQ ID NO: 219)
SFWLATIER;
and
(SEQ ID NO: 220)
WLATIER,

and naturally-occurring variants of any of the foregoing.

### Collagen Alpha-5(IV) Proteolytic Fragments

Non-limiting examples of enzymatic cleavage products (enzymatic cleavage products generated by an enzyme produced by a cell (e.g., an inflammatory cell) in the vasculature) of a Collagen alpha-5(IV) chain, listed in sequence order, include:

(SEQ ID NO: 221)
DGIPGPPGPK;
(SEQ ID NO: 222)
KGNPGYPGPPGIQGLPGPTGIPGPIGPPGPPGLMGPPGPPGLPGPK;
(SEQ ID NO: 223)
PHIPPSDEICEPGPPGPPGSPGDK;
(SEQ ID NO: 224)
GLPGLPGPPGSLGFPGQK;
(SEQ ID NO: 225)
PKGEPGGITFK;
(SEQ ID NO: 226)
TPGRIGLEGPPGPPGFPGPK;
(SEQ ID NO: 227)
GPPGRTGLDGLPGPK;
(SEQ ID NO: 228)
APGPIGPPGSPGLPGK;
(SEQ ID NO: 229)
KGEPGLPGPPGPMDPNLLGSK;
(SEQ ID NO: 230)
PGEPGPVGGGGHPGQPGPPGEK;
(SEQ ID NO: 231)
PALEGPKGNPGPQGPPGRPGPTGFQGLPGPEGPPGLPGNGGIK;
and
(SEQ ID NO: 232)
PPGPPGLPGPSGQSIIIK,

and naturally-occurring variants of any of the foregoing.

### Collagen Alpha-1(XV) Proteolytic Fragments

Non-limiting examples of enzymatic cleavage products (enzymatic cleavage products generated by an enzyme produced by a cell (e.g., an inflammatory cell) in the vasculature) of a Collagen alpha-1(XV) chain, listed in sequence order, include:

(SEQ ID NO: 233)
VDGATGLPGMK;
(SEQ ID NO: 234)
KGQAGPPGVMGPPGPPGPPGPPGPGCTMGLGFED;
(SEQ ID NO: 235)
KLQLGELIPIPADSPPPP;
(SEQ ID NO: 236)
AWRTADTAVTGLASPLSTGK;
and
(SEQ ID NO: 237)
AVTGLASPLSTGKILDQK,

and naturally-occurring variants of any of the foregoing.

### Collagen Alpha-3(VI) Proteolytic Fragments

Non-limiting examples of enzymatic cleavage products (enzymatic cleavage products generated by an enzyme produced by a cell (e.g., an inflammatory cell) in the vasculature) of a Collagen alpha-3(VI) chain, listed in sequence order, include:

(SEQ ID NO: 238)
GVEDADEGALKEIASEPLNMHMFNLENFTSLHDIVGNLVSCVHSSVSPER;
(SEQ ID NO: 239)
NNLFTSSAGYR;
(SEQ ID NO: 240)
AAPLQGMLPGLLAPLR;
and
(SEQ ID NO: 241)
IGDLHPQIVN,

and naturally-occurring variants thereof.

### Collagen Alpha-1(VI) Proteolytic Fragments

Non-limiting examples of enzymatic cleavage products (enzymatic cleavage products generated by an enzyme produced by a cell (e.g., an inflammatory cell) in the vasculature) of a Collagen alpha-1(VI) chain, listed in sequence order, include:

(SEQ ID NO: 242)
GPQGDQGR;
(SEQ ID NO: 243)
TDPAHDVR;
(SEQ ID NO: 244)
FSDGNSQGATPAAIEK;
(SEQ ID NO: 245)
QVNEPHIR;
and
(SEQ ID NO: 246)
GVFHQTVSR,

and naturally-occurring variants thereof.

### Collagen Alpha-1(XIX) Proteolytic Fragments

Non-limiting examples of enzymatic cleavage products (enzymatic cleavage products generated by an enzyme produced by a cell (e.g., an inflammatory cell) in the vasculature) of a Collagen alpha-1(XIX) chain, include, e.g., NPGAPGPR (SEQ ID NO:355), and naturally-occurring variants thereof.

### Fibronectin

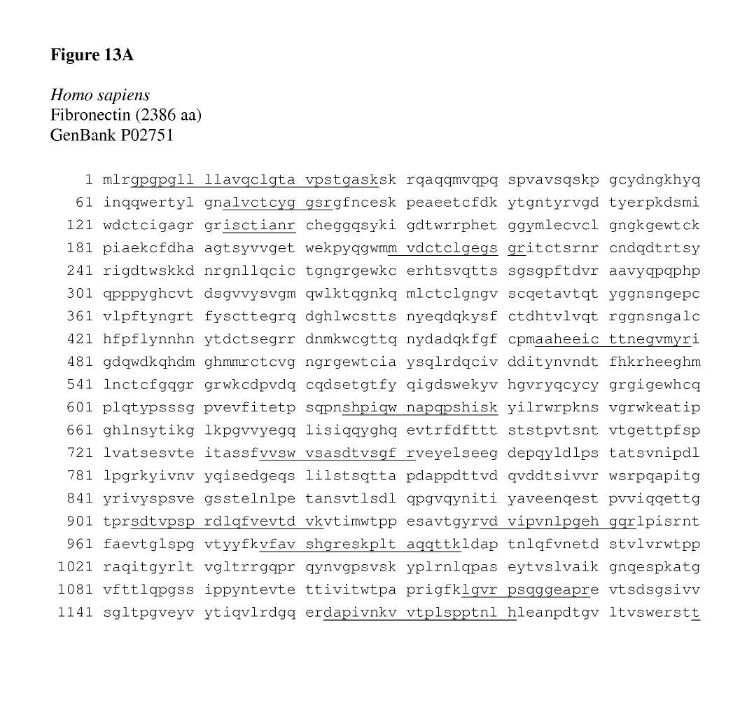

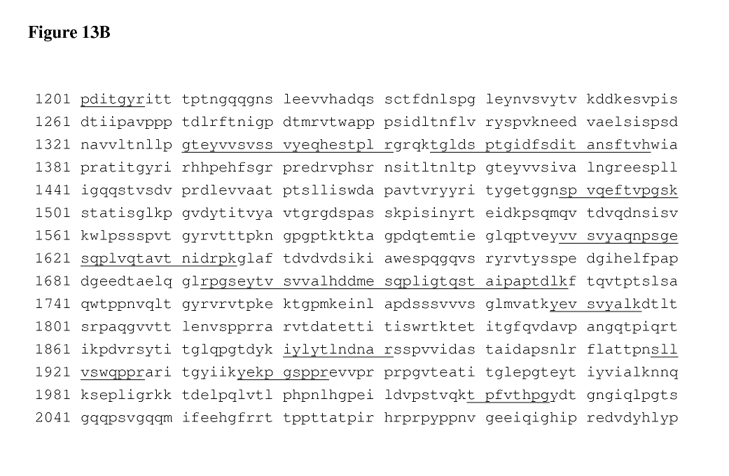

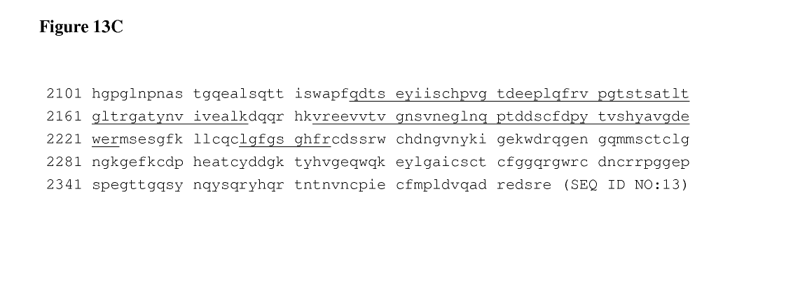

An enzymatic cleavage product of a structural component of an arteriosclerotic plaque includes an enzymatic cleavage product of fibronectin. For example, an enzymatic cleavage product of a structural component of an arteriosclerotic plaque includes an enzymatic cleavage product of a fibronectin polypeptide having at least about 98%, at least about 99%, or 100%, amino acid sequence identity with an amino acid sequence of a fibronectin polypeptide depicted in FIGS. 13A-C. An enzymatic cleavage product of a fibronectin component of an arteriosclerotic plaque can be an enzymatic cleavage product of a naturally-occurring variant (polymorphism) of a fibronectin polypeptide depicted in FIGS. 13A-C.

Non-limiting examples of enzymatic cleavage products (enzymatic cleavage products generated by an enzyme produced by a cell (e.g., an inflammatory cell) in the vasculature) of fibronectin, listed in sequence order, include:

(SEQ ID NO: 247)
GPGLLLLAVQCLGTAVPSTGASK;
(SEQ ID NO: 248)
ALVCTCYGGSR;
(SEQ ID NO: 327)
ISCTIANR;
(SEQ ID NO: 249)
MVDCTCLGEGSGR;
(SEQ ID NO: 250)
AAHEEICTTNEGVMYR;
(SEQ ID NO: 251)
SHPIQWNAPQPSHISK;
(SEQ ID NO: 252)
VVSWVSASDTVSGFR;
(SEQ ID NO: 253)
SDTVPSPRDLQFVEVTDVK;
(SEQ ID NO: 254)
VDVIPVNLPGEHGQR;
(SEQ ID NO: 255)
VFAVSHGRESKPLTAQQTTK;
(SEQ ID NO: 256)
LGVRPSQGGEAPR;
(SEQ ID NO: 257)
DAPIVNKVVTPLSPPTNLH;
(SEQ ID NO: 258)
TPDITGYR;
(SEQ ID NO: 259)
PGTEYVVSVSSVYEQHESTPLR;
(SEQ ID NO: 260)
TGLDSPTGIDFSDITANSFTVH;
(SEQ ID NO: 261)
TVHWIAPR;
(SEQ ID NO: 262)
SPVQEFTVPGSK;
(SEQ ID NO: 263)
VVSVYAQNPSGESQPLVQTAVTNIDRPK;
(SEQ ID NO: 264)
RPGSEYTVSVVALHDDMESQPLIGTQSTAIPAPTDLK;
(SEQ ID NO: 265)
YEVSVYALK;
(SEQ ID NO: 266)
IYLYTLNDNAR;
(SEQ ID NO: 267)
SLLVSWQPPR;
(SEQ ID NO: 268)
YEKPGSPPR;
(SEQ ID NO: 269)
TPFVTHPG;
(SEQ ID NO: 270)
TPFVTHPGYDT;
(SEQ ID NO: 271)
TPFVTHPGYDTGNGIQLPGTSGQQPSVGQQM;
(SEQ ID NO: 272)
QDTSEYIISCHPVGTDEEPLQFR;
(SEQ ID NO: 273)
VPGTSTSATLTGLTRGATYNIIVEALK;
(SEQ ID NO: 274)
VREEVVTVGN;
(SEQ ID NO: 275)
SVNEGLNQPTDDSCFDPYTVSHYAVGDEWER;
and
(SEQ ID NO: 276)
LGFGSGHFR,

and naturally-occurring variants of any of the foregoing.

### Fibrillin

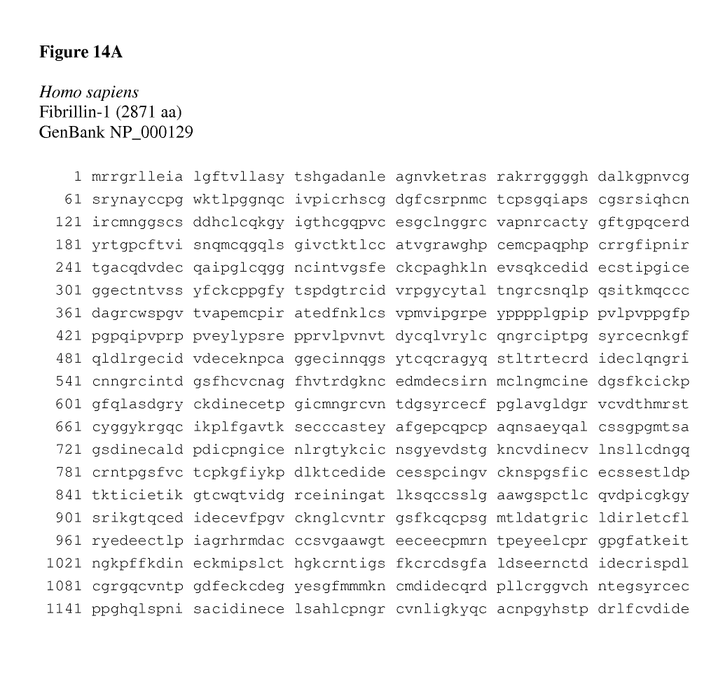

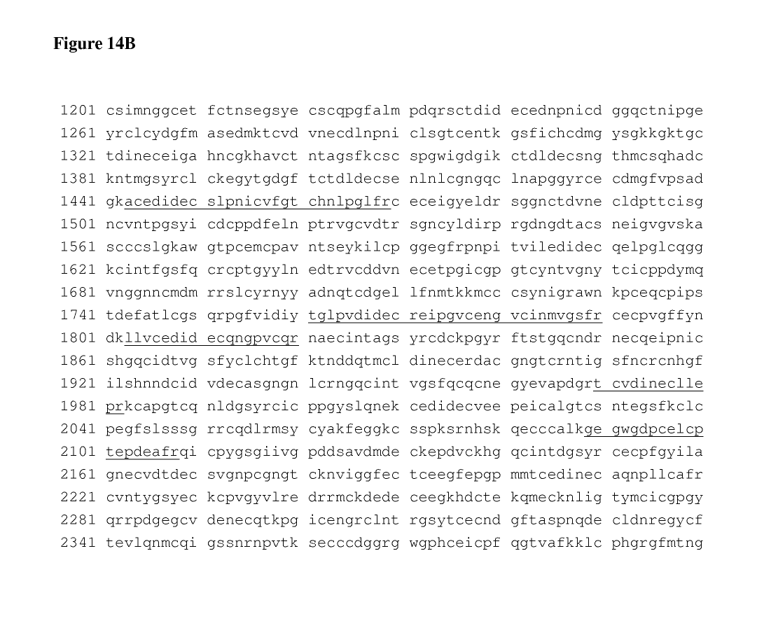

An enzymatic cleavage product of a structural component of an arteriosclerotic plaque includes an enzymatic cleavage product of fibrillin, e.g., fibrillin-1. For example, an enzymatic cleavage product of a structural component of an arteriosclerotic plaque includes an enzymatic cleavage product of a fibrillin-1 polypeptide having at least about 98%, at least about 99%, or 100%, amino acid sequence identity with an amino acid sequence of a fibrillin-1 polypeptide depicted in FIGS. 14A-C. An enzymatic cleavage product of a fibrillin-1 component of an arteriosclerotic plaque can be an enzymatic cleavage product of a naturally-occurring variant (polymorphism) of a fibrillin-1 polypeptide depicted in FIGS. 14A-C.

Non-limiting examples of enzymatic cleavage products (enzymatic cleavage products generated by an enzyme produced by a cell (e.g., an inflammatory cell) in the vasculature) of fibrillin, listed in sequence order, include:

(SEQ ID NO: 277)
ACEDIDECSLPNICVFGTCHNLPGLFR;
(SEQ ID NO: 278)
TGLPVDIDECR;
(SEQ ID NO: 279)
PVDIDECR;
(SEQ ID NO: 280)
EIPGVCNGVCINHVGSFR;
(SEQ ID NO: 281)
EIPGVCENGVCINMVGSFR;
(SEQ ID NO: 282)
LLVCEDIDECQNGPVCQR;
(SEQ ID NO: 283)
TCVDINECLLEPR;
(SEQ ID NO: 284)
GEGWGDPCELCPTEPDEAFR,
and

and naturally-occurring variants thereof.

### Vitronectin

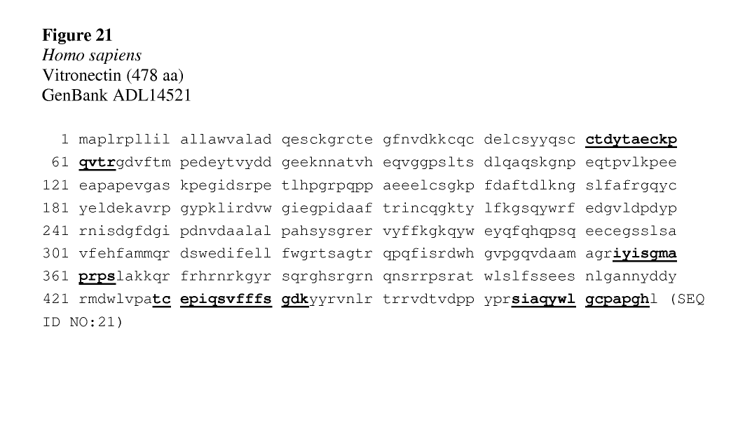

An enzymatic cleavage product of a structural component of an arteriosclerotic plaque includes an enzymatic cleavage product of vitronectin. For example, an enzymatic cleavage product of a structural component of an arteriosclerotic plaque includes an enzymatic cleavage product of a vitronectin polypeptide having at least about 98%, at least about 99%, or 100%, amino acid sequence identity with an amino acid sequence of a vitronectin polypeptide depicted in FIG. 21. An enzymatic cleavage product of a vitronectin component of an arteriosclerotic plaque can be an enzymatic cleavage product of a naturally-occurring variant (polymorphism) of a vitronectin polypeptide depicted in FIG. 21.

Non-limiting examples of enzymatic cleavage products (enzymatic cleavage products generated by an enzyme produced by a cell (e.g., an inflammatory cell) in the vasculature) of vitronectin, listed in sequence order, include:

(SEQ ID NO: 285)
CTDYTAECKPQVTR;
(SEQ ID NO: 286)
IYISGMAPRPS;
(SEQ ID NO: 287)
TCEPIQSVFFFSGDK;
(SEQ ID NO: 288)
SIAQYWLGCPAPGH;
and
(SEQ ID NO: 289)
WLGCPAPGHL,

and naturally-occurring variants of any of the foregoing.

### Metalloproteinase Inhibitor-1

An enzymatic cleavage product of a structural component of an arteriosclerotic plaque includes an enzymatic cleavage product of metalloproteinase inhibitor-1. For example, an enzymatic cleavage product of a structural component of an arteriosclerotic plaque includes an enzymatic cleavage product of a metalloproteinase inhibitor-1polypeptide having at least about 98%, at least about 99%, or 100%, amino acid sequence identity with an amino acid sequence of a metalloproteinase inhibitor-1polypeptide depicted in FIG. 16. An enzymatic cleavage product of a metalloproteinase inhibitor-1component of an arteriosclerotic plaque can be an enzymatic cleavage product of a naturally-occurring variant (polymorphism) of a metalloproteinase inhibitor-1polypeptide depicted in FIG. 16.

Non-limiting examples of enzymatic cleavage products (enzymatic cleavage products generated by an enzyme produced by a cell (e.g., an inflammatory cell) in the vasculature) of metalloproteinase inhibitor-1, listed in sequence order, include:

(SEQ ID NO: 290)
CNSDLVIR;
(SEQ ID NO: 291)
LQDGLLHITTC;
and
(SEQ ID NO: 292)
SFVAPWNSLSLAQR,

and naturally-occurring variants of any of the foregoing.

### Dermatopontin

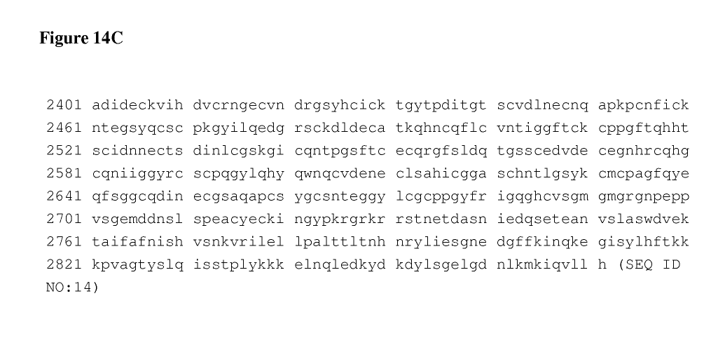

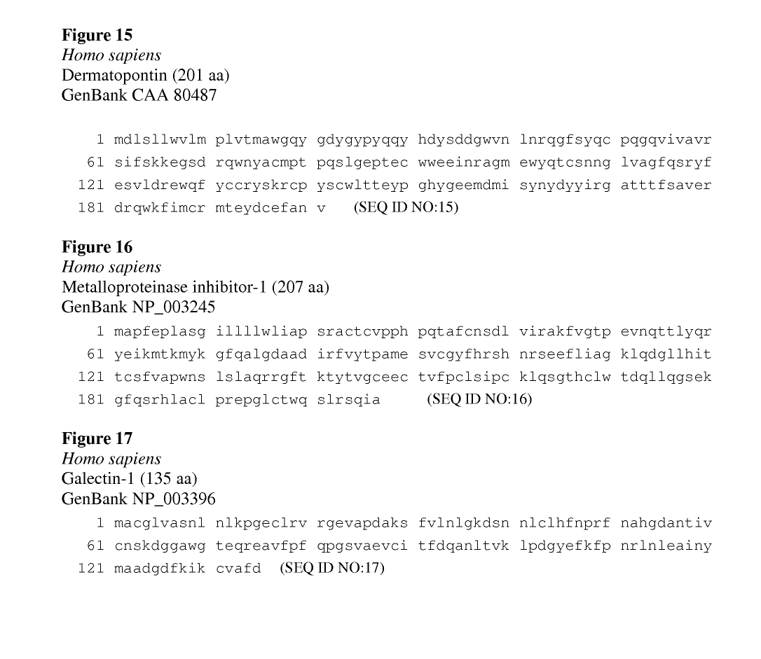

An enzymatic cleavage product of a structural component of an arteriosclerotic plaque includes an enzymatic cleavage product of dermatopontin. For example, an enzymatic cleavage product of a structural component of an arteriosclerotic plaque includes an enzymatic cleavage product of a dermatopontin polypeptide having at least about 98%, at least about 99%, or 100%, amino acid sequence identity with an amino acid sequence of a dermatopontin polypeptide depicted in FIG. 15. An enzymatic cleavage product of a dermatopontin component of an arteriosclerotic plaque can be an enzymatic cleavage product of a naturally-occurring variant (polymorphism) of a dermatopontin polypeptide depicted in FIG. 15.

Non-limiting examples of enzymatic cleavage products (enzymatic cleavage products generated by an enzyme produced by a cell (e.g., an inflammatory cell) in the vasculature) of dermatopontin, listed in sequence order, include:

(SEQ ID NO: 293)
SDDGWVNLNR;
(SEQ ID NO: 294)
SYQCPQGQVIVAVR;
(SEQ ID NO: 295)
SLGEPTECWWEEINR;
and
(SEQ ID NO: 296)
SNNGLVAGFQSR,

and naturally-occurring variants of any of the foregoing.

### Galectin-1

An enzymatic cleavage product of a structural component of an arteriosclerotic plaque includes an enzymatic cleavage product of galectin-1. For example, an enzymatic cleavage product of a structural component of an arteriosclerotic plaque includes an enzymatic cleavage product of a galectin-1 polypeptide having at least about 98%, at least about 99%, or 100%, amino acid sequence identity with an amino acid sequence of a galectin-1 polypeptide depicted in FIG. 17. An enzymatic cleavage product of a galectin-1 component of an arteriosclerotic plaque can be an enzymatic cleavage product of a naturally-occurring variant (polymorphism) of a galectin-1 polypeptide depicted in FIG. 17.

Non-limiting examples of enzymatic cleavage products (enzymatic cleavage products generated by an enzyme produced by a cell (e.g., an inflammatory cell) in the vasculature) of galectin-1, listed in sequence order, include:

(SEQ ID NO: 297)
VASNLNLKPGECLR;
(SEQ ID NO: 298)
GDANTIVCNSK;
and
(SEQ ID NO: 299)
MAADGDFK,

and naturally-occurring variants of any of the foregoing.

### Lumican

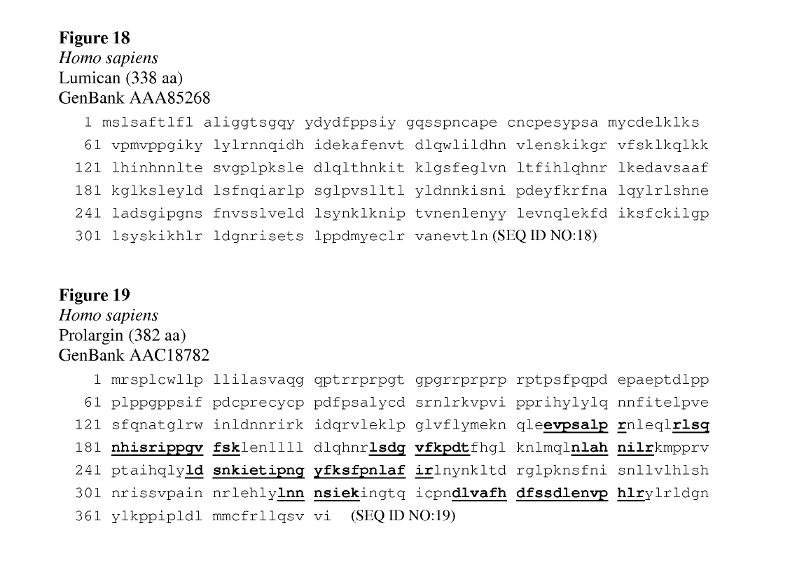

An enzymatic cleavage product of a structural component of an arteriosclerotic plaque includes an enzymatic cleavage product of lumican. For example, an enzymatic cleavage product of a structural component of an arteriosclerotic plaque includes an enzymatic cleavage product of a lumican polypeptide having at least about 98%, at least about 99%, or 100%, amino acid sequence identity with an amino acid sequence of a lumican polypeptide depicted in FIG. 18. An enzymatic cleavage product of a lumican component of an arteriosclerotic plaque can be an enzymatic cleavage product of a naturally-occurring variant (polymorphism) of a lumican polypeptide depicted in FIG. 18. In some cases, an enzymatic cleavage product of lumican is specifically excluded.

Non-limiting examples of enzymatic cleavage products (enzymatic cleavage products generated by an enzyme produced by a cell (e.g., an inflammatory cell) in the vasculature) of lumican, listed in sequence order, include:

(SEQ ID NO: 300)
CAPECNCPESYPSAMYCDELK;
(SEQ ID NO: 301)
RNNQIDHIDEK;
(SEQ ID NO: 302)
NNQIDHIDE;
(SEQ ID NO: 303)
ILDHNLLENSK;
(SEQ ID NO: 304)
SLEDLQLTH;
(SEQ ID NO: 305)
IHLQHNR;
and
(SEQ ID NO: 306)
CKILGPLSYSK,

and naturally-occurring variants of any of the foregoing.

### Prolargin

An enzymatic cleavage product of a structural component of an arteriosclerotic plaque includes an enzymatic cleavage product of prolargin. For example, an enzymatic cleavage product of a structural component of an arteriosclerotic plaque includes an enzymatic cleavage product of a prolargin polypeptide having at least about 98%, at least about 99%, or 100%, amino acid sequence identity with an amino acid sequence of a prolargin polypeptide depicted in FIG. 19. An enzymatic cleavage product of a prolargin component of an arteriosclerotic plaque can be an enzymatic cleavage product of a naturally-occurring variant (polymorphism) of a prolargin polypeptide depicted in FIG. 19.

Non-limiting examples of enzymatic cleavage products (enzymatic cleavage products generated by an enzyme produced by a cell (e.g., an inflammatory cell) in the vasculature) of prolargin, listed in sequence order, include:

(SEQ ID NO: 307)
EVPSALPR;
(SEQ ID NO: 308)
RLSQNHISR;
(SEQ ID NO: 309)
RLSQNHISRIPPGVFSK;
(SEQ ID NO: 310)
LSDGVFKPDT;
(SEQ ID NO: 311)
NLAHNILR;
(SEQ ID NO: 312)
LAHNILR;
(SEQ ID NO: 313)
LDSNKIETIPNGYFKSFPNLAFIR;
(SEQ ID NO: 314)
IETIPNGYFKSFPNLA;
(SEQ ID NO: 315)
SFPNLAFIRLNYN;
(SEQ ID NO: 316)
LNNNSIEK;
and
(SEQ ID NO: 317)
DLVAFHDFSSDLENVPHLR,

and naturally-occurring variants of any of the foregoing.

### Tenascin

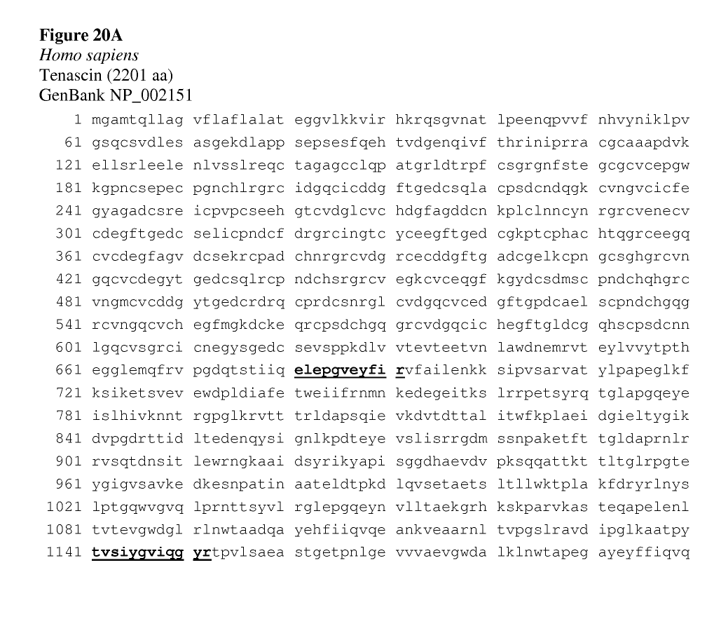

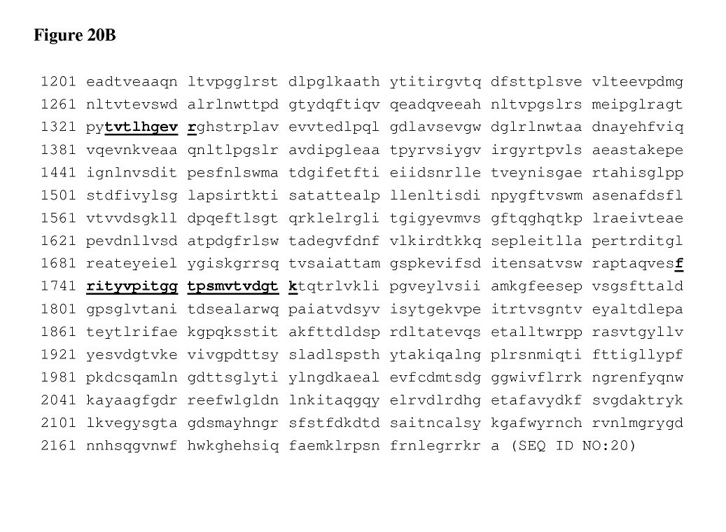

An enzymatic cleavage product of a structural component of an arteriosclerotic plaque includes an enzymatic cleavage product of tenascin. For example, an enzymatic cleavage product of a structural component of an arteriosclerotic plaque includes an enzymatic cleavage product of a tenascin polypeptide having at least about 98%, at least about 99%, or 100%, amino acid sequence identity with an amino acid sequence of a tenascin polypeptide depicted in FIGS. 20A and 20B. An enzymatic cleavage product of a tenascin component of an arteriosclerotic plaque can be an enzymatic cleavage product of a naturally-occurring variant (polymorphism) of a tenascin polypeptide depicted in FIGS. 20A and 20B.

Non-limiting examples of enzymatic cleavage products (enzymatic cleavage products generated by an enzyme produced by a cell (e.g., an inflammatory cell) in the vasculature) of tenascin, listed in sequence order, include:

(SEQ ID NO: 318)
ELEPGVEYFIR;
(SEQ ID NO: 319)
TVSIYGVIQGYR;
(SEQ ID NO: 320)
TVTLHGEVR;
and
(SEQ ID NO: 321)
FRITYVPITGGTPSMVTVDGTK,

and naturally-occurring variants of any of the foregoing.

### Tenascin-X

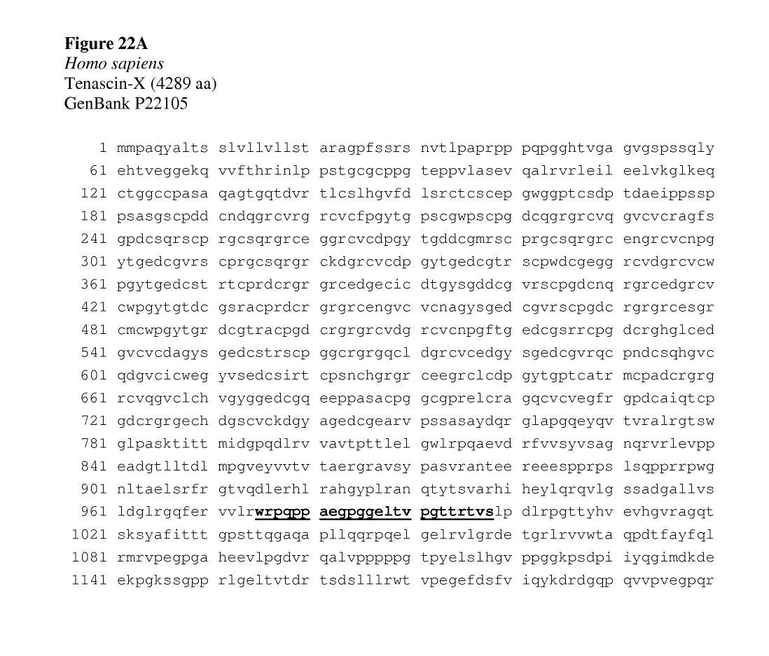

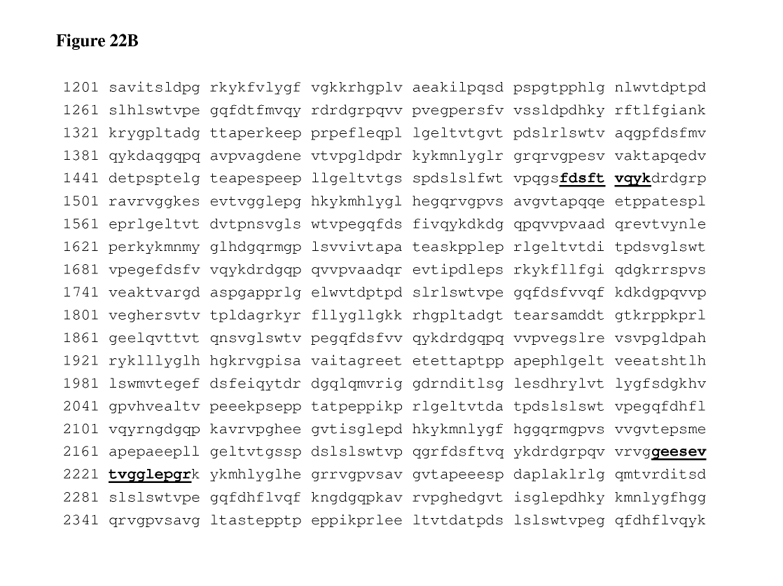

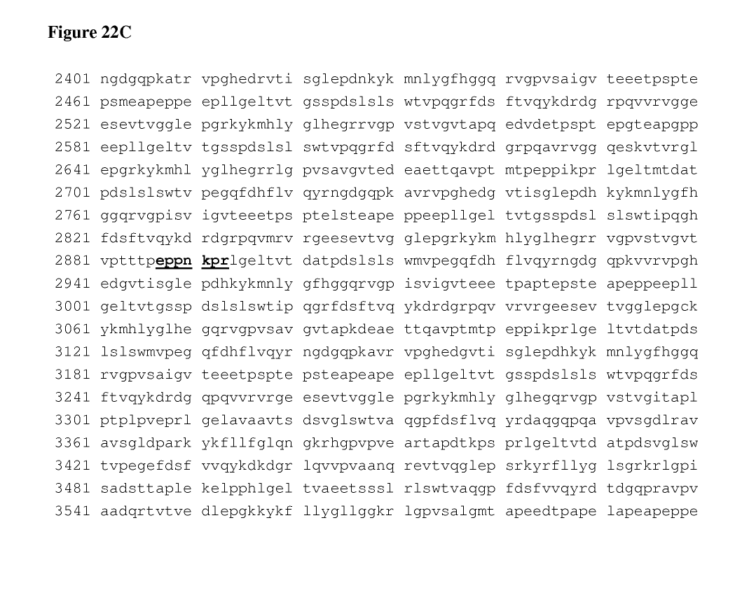

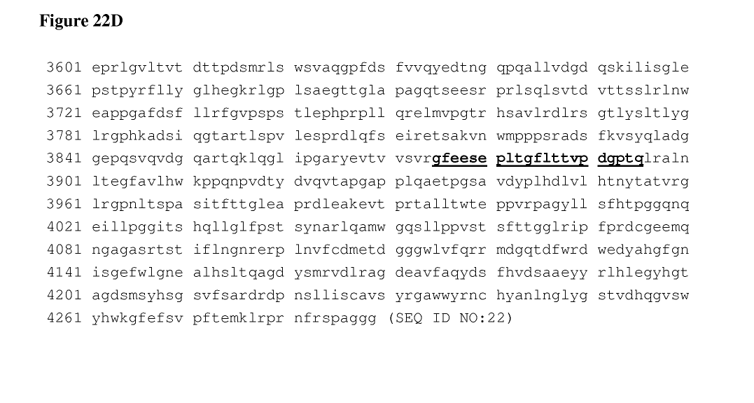

An enzymatic cleavage product of a structural component of an arteriosclerotic plaque includes an enzymatic cleavage product of tenascin-X. For example, an enzymatic cleavage product of a structural component of an arteriosclerotic plaque includes an enzymatic cleavage product of a tenascin-X polypeptide having at least about 98%, at least about 99%, or 100%, amino acid sequence identity with an amino acid sequence of a tenascin-X polypeptide depicted in FIGS. 22A-D. An enzymatic cleavage product of a tenascin-X component of an arteriosclerotic plaque can be an enzymatic cleavage product of a naturally-occurring variant (polymorphism) of a tenascin-X polypeptide depicted in FIGS. 22A-D.

Non-limiting examples of enzymatic cleavage products (enzymatic cleavage products generated by an enzyme produced by a cell (e.g., an inflammatory cell) in the vasculature) of tenascin-X, listed in sequence order, include:

(SEQ ID NO: 322)
WRPQPPAEGPGGELTVPGTTRTVS;
(SEQ ID NO: 323)
FDSFTVQYK;
(SEQ ID NO: 324)
GEESEVTVGGLEPGR;
(SEQ ID NO: 325)
EPPNKPR;
and
(SEQ ID NO: 326)
GFEESEPLTGFLTTVPDGPTQ,

and naturally-occurring variants of any of the foregoing.

In some embodiments, any one or more of the above-listed fragments is specifically excluded.

### Panels

The present disclosure provides a panel of enzymatic cleavage products (enzymatic cleavage products generated by an enzyme produced by a cell (e.g., an inflammatory cell) in the vasculature). The panel can include 2, 3, 4, 5, 6, 7, 8, 9, 10, 10-15, 15-20, 20-25, 25-30, 30-35, 35-40, 40-45, 45-50, or more than 50, of the above-described enzymatic cleavage products. Thus, the panel can include 2, 3, 4, 5, 6, 7, 8, 9, 10, 10-15, 15-20, 20-25, 25-30, 30-35, 35-40, 40-45, 45-50, or more than 50, different enzymatic cleavage products (peptides), selected from the above-described enzymatic cleavage products. Enzymatic cleavage products in the panel can be purified.

As one non-limiting example, a subject peptide panel includes:

1) one or more fibrillin fragments selected from:

(SEQ ID NO: 277)
ACEDIDECSLPNICVFGTCHNLPGLFR;
(SEQ ID NO: 278)
TGLPVDIDECR;
(SEQ ID NO: 279)
PVDIDECR;
(SEQ ID NO: 280)
EIPGVCNGVCINHVGSFR;
(SEQ ID NO: 281)
EIPGVCENGVCINMVGSFR;
(SEQ ID NO: 282)
LLVCEDIDECQNGPVCQR;
(SEQ ID NO: 283)
TCVDINECLLEPR;
(SEQ ID NO: 284)
GEGWGDPCELCPTEPDEAFR;
and

and naturally-occurring variants thereof;

2) one or more vitronectin fragments selected from:

(SEQ ID NO: 285)
CTDYTAECKPQVTR;
(SEQ ID NO: 286)
IYISGMAPRPS;
(SEQ ID NO: 287)
TCEPIQSVFFFSGDK;
(SEQ ID NO: 288)
SIAQYWLGCPAPGH;
and
(SEQ ID NO: 289)
WLGCPAPGHL,

and naturally-occurring variants of any of the foregoing;

3) one or more fibronectin fragments selected from:

(SEQ ID NO: 247)
GPGLLLLAVQCLGTAVPSTGASK;
(SEQ ID NO: 248)
ALVCTCYGGSR;
(SEQ ID NO: 327)
ISCTIANR;
(SEQ ID NO: 249)
MVDCTCLGEGSGR;
(SEQ ID NO: 250)
AAHEEICTTNEGVMYR;
(SEQ ID NO: 251)
SHPIQWNAPQPSHISK;
(SEQ ID NO: 252)
VVSWVSASDTVSGFR;
(SEQ ID NO: 253)
SDTVPSPRDLQFVEVTDVK;
(SEQ ID NO: 254)
VDVIPVNLPGEHGQR;
(SEQ ID NO: 255)
VFAVSHGRESKPLTAQQTTK;
(SEQ ID NO: 256)
LGVRPSQGGEAPR;
(SEQ ID NO: 257)
DAPIVNKVVTPLSPPTNLH;
(SEQ ID NO: 258)
TPDITGYR;
(SEQ ID NO: 259)
PGTEYVVSVSSVYEQHESTPLR;
(SEQ ID NO: 260)
TGLDSPTGIDFSDITANSFTVH;
(SEQ ID NO: 261)
TVHWIAPR;
(SEQ ID NO: 262)
SPVQEFTVPGSK;
(SEQ ID NO: 263)
VVSVYAQNPSGESQPLVQTAVTNIDRPK;
(SEQ ID NO: 264)
RPGSEYTVSVVALHDDMESQPLIGTQSTAIPAPTDLK;
(SEQ ID NO: 265)
YEVSVYALK;
(SEQ ID NO: 266)
IYLYTLNDNAR;
(SEQ ID NO: 267)
SLLVSWQPPR;
(SEQ ID NO: 268)
YEKPGSPPR;
(SEQ ID NO: 269)
TPFVTHPG;
(SEQ ID NO: 270)
TPFVTHPGYDT;
(SEQ ID NO: 271)
TPFVTHPGYDTGNGIQLPGTSGQQPSVGQQM;
(SEQ ID NO: 272)
QDTSEYIISCHPVGTDEEPLQFR;
(SEQ ID NO: 273)
VPGTSTSATLTGLTRGATYNIIVEALK;
(SEQ ID NO: 274)
VREEVVTVGN;
(SEQ ID NO: 275)
SVNEGLNQPTDDSCFDPYTVSHYAVGDEWER;
and
(SEQ ID NO: 276)
LGFGSGHFR,

and naturally-occurring variants of any of the foregoing.

A subject panel can further include:

4) one or more tenascin peptides selected from:

(SEQ ID NO: 318)
ELEPGVEYFIR;
(SEQ ID NO: 319)
TVSIYGVIQGYR;
(SEQ ID NO: 320)
TVTLHGEVR;
and
(SEQ ID NO: 321)
FRITYVPITGGTPSMVTVDGTK,

and naturally-occurring variants of any of the foregoing;

5) one or more prolargin peptides selected from:

(SEQ ID NO: 307)
EVPSALPR;
(SEQ ID NO: 308)
RLSQNHISR;
(SEQ ID NO: 309)
RLSQNHISRIPPGVFSK;
(SEQ ID NO: 310)
LSDGVFKPDT;
(SEQ ID NO: 311)
NLAHNILR;
(SEQ ID NO: 312)
LAHNILR;
(SEQ ID NO: 313)
LDSNKIETIPNGYFKSFPNLAFIR;
(SEQ ID NO: 314)
IETIPNGYFKSFPNLA;
(SEQ ID NO: 315)
SFPNLAFIRLNYN;
(SEQ ID NO: 316)
LNNNSIEK;
and
(SEQ ID NO: 317)
DLVAFHDFSSDLENVPHLR,

and naturally-occurring variants of any of the foregoing; and

6) one or more dermatopontin peptides selected from:

(SEQ ID NO: 293)
SDDGWVNLNR;
(SEQ ID NO: 294)
SYQCPQGQVIVAVR;
(SEQ ID NO: 295)
SLGEPTECWWEEINR;
and
(SEQ ID NO: 296)
SNNGLVAGFQSR,

and naturally-occurring variants of any of the foregoing.

A subject method can involve detecting peptides present in a subject panel. In some embodiments, a subject panel can serve as a control.

In some embodiments, the enzymatic cleavage products of a subject panel are immobilized on an insoluble support. In some embodiments, the enzymatic cleavage products of a subject panel are detectably labeled.

### Further Processing In Vitro

In some cases, an enzymatic cleavage product generated in the vasculature (e.g., in vivo) is further processed in vitro. In vitro processing of an in vivo-generated enzymatic cleavage product can include enzymatic digestion in vitro. Thus, in some cases, an enzymatic cleavage product generated in the vasculature (e.g., in vivo) is further cleaved enzymatically, e.g., in vitro. Such in vitro cleavage of a cleavage product produced in vivo may be undertaken in order to characterize an in vivo-generated cleavage product. As an example, an enzymatic cleavage product of a structural component of an unstable arteriosclerotic plaque, generated by an enzyme produced by an inflammatory cell in the vasculature, may be subjected to trypsin digestion or other enzymatic digestion in vitro before the cleavage product is analyzed by mass spectrometry (MS). In vitro enzymatic cleavage can include trypsinization.

Non-limiting examples of trypsin cleavage products of an enzymatic cleavage products of a collagen alpha-1 (VI) component of an arteriosclerotic plaque include:

1)
(SEQ ID NO: 328)
LFSDGNSQGATPAAIEKA;
2)
(SEQ ID NO: 329)
RGVFHQTVSRK;
3)
(SEQ ID NO: 330)
RQVNEPHIRV;
and
4)
(SEQ ID NO: 331)
RTDPAHDVRV,

and naturally-occurring variants of any of the foregoing.

A non-limiting example of enzymatic cleavage products of a collagen alpha-3(VI) component of an arteriosclerotic plaque include: IGDLHPQIVN (SEQ ID NO:241).

Non-limiting examples of enzymatic cleavage products of fibronectin include, e.g.:

1)
(SEQ ID NO: 332)
DAPIVNK;
2)
(SEQ ID NO: 333)
SEPLIGR;
3)
(SEQ ID NO: 334)
ATITGYR;
4)
(SEQ ID NO: 335)
AQITGYR;
5)
(SEQ ID NO: 336)
SDTVPSPR;
6)
(SEQ ID NO: 337)
VFAVSHGR;
7)
(SEQ ID NO: 338)
ISCTIANRC;
8)
(SEQ ID NO: 339)
PLTAQQTTK;
9)
(SEQ ID NO: 340)
YEVSVYALK;
10)
(SEQ ID NO: 341)
QYNVGPSVSK;
11)
(SEQ ID NO: 342)
GATYNIIVEALK;
12)
(SEQ ID NO: 343)
DLQFVEVTDVK;
13)
(SEQ ID NO: 344)
LGVRPSQGGEAPR;
14)
(SEQ ID NO: 345)
IYLYTLNDNAR;
15)
(SEQ ID NO: 346)
VPGTSTSATLTGLTR;
and
16)
(SEQ ID NO: 347)
VDVIPVNLPGEHGQR,

and naturally-occurring variants thereof.

Non-limiting examples of enzymatic cleavage products of fibrillin-1 include, e.g.:

1)
(SEQ ID NO: 348)
KACEDIDECSLPNICVFGTCHNLPGLFRC;
2)
(SEQ ID NO: 349)
YTGLPVDIDECRE;
3)
(SEQ ID NO: 350)
LPVDIDECRE;
4)
(SEQ ID NO: 351)
REIPGVCENGVCINMVGSFRC;
5)
(SEQ ID NO: 352)
KLLVCEDIDECQNGPVCQRN;
6)
(SEQ ID NO: 353)
RTCVDINECLLEPRK;
and
7)
(SEQ ID NO: 354)
KGEGWGDPCELCPTEPDEAFRQ,

and naturally-occurring variants thereof.

### Detection Methods

An enzymatic cleavage product of a protein component of an arteriosclerotic plaque can be detected using any known method, where suitable methods include, e.g., immunological methods, gel electrophoresis methods, chromatographic methods, and mass spectrometric methods.

### Immunological Methods

Suitable immunological methods include, e.g., enzyme-linked immunosorbent assay (ELISA), radioimmunoassay (RIA), and an immunofixation assay. Immunological assays involve use of antibody specific for an enzymatic cleavage product of a protein component of an arteriosclerotic plaque. In some instances, the primary antibody used an immunological assay is an antibody specific for an epitope that is exposed upon cleavage of a protein component of an arteriosclerotic plaque, e.g., an epitope that is not accessible to the antibody in the protein in its uncleaved state. For example, an immunological assay can involve use of antibody specific for an epitope that is exposed upon cleavage of a protein component of an arteriosclerotic plaque, e.g., an epitope comprising the newly-formed carboxyl-terminus or amino-terminus of an enzymatic cleavage product of a protein component of an arteriosclerotic plaque. For example, an enzyme(s) produced by an inflammatory cell in the vasculature can proteolytically cleave a protein component of an arteriosclerotic plaque; and the cleavage product would then present epitope(s) (e.g., linear epitopes; conformational epitopes) not presented by the uncleaved protein component, where such epitopes could include the C-terminal amino acids of the cleavage product, or the N-terminal amino acids of the cleavage product.

In some instances, the antibody used in an immunological assay is immobilized on an insoluble (e.g., a solid) support. Suitable supports are well known in the art and comprise, inter alia, commercially available column materials, polystyrene beads, latex beads, magnetic beads, colloid metal particles, glass and/or silicon chips and surfaces, nitrocellulose strips, nylon membranes, sheets, duracytes, wells of reaction trays (e.g., multi-well plates), test strips, plastic tubes, etc. A solid support can comprise any of a variety of substances, including, e.g., glass, polystyrene, polyvinyl chloride, polypropylene, polyethylene, polycarbonate, dextran, nylon, amylose, natural and modified celluloses, polyacrylamides, agaroses, and magnetite. Suitable methods for immobilizing an antibody onto a solid support are well known and include, but are not limited to ionic, hydrophobic, covalent interactions and the like. Solid supports can be soluble or insoluble, e.g., in aqueous solution. In some embodiments, a suitable solid support is generally insoluble in an aqueous solution.

In some instances, antibody used in an immunological assay comprises a detectable label. Suitable detectable labels include any composition detectable by spectroscopic, photochemical, biochemical, immunochemical, electrical, optical or chemical means. Suitable include, but are not limited to, magnetic beads (e.g. Dynabeads™), fluorescent dyes (e.g., fluorescein isothiocyanate, texas red, rhodamine, a green fluorescent protein, a red fluorescent protein, a yellow fluorescent protein, and the like), radiolabels (e.g., ³H, ¹²⁵I, ³⁵S, ¹⁴C, or ³²P), enzymes (e.g., horse radish peroxidase, alkaline phosphatase, luciferase, and others commonly used in an enzyme-linked immunosorbent assay (ELISA)), and colorimetric labels such as colloidal gold or colored glass or plastic (e.g. multistyrene, multipropylene, latex, etc.) beads.

### Mass Spectrometric Methods

In some cases, a detection method provides size information about an enzymatic cleavage product. Detection methods that provide size information include, e.g., gel electrophoresis methods, and the like. In some cases, a detection method provides size information about an enzymatic cleavage product; and also involves use of an antibody specific for the enzymatic cleavage product.

In some instances, identification of cleavage products is carried out using mass spectrometry. For example, as discussed above, an in vivo-generated enzymatic cleavage product can be subjected to enzymatic digestion in vitro using a specific protease (e.g. trypsin), followed by detection and/or quantitation, of specific peptide products of the in vivo-generated enzymatic cleavage product. Mass spectrometric detection approaches include detection of peptide masses, detection of masses of fragments formed in the mass spectrometer, or a combination of the foregoing (e.g. Selective Reaction Monitoring).

### Controls

Levels of an enzymatic cleavage product of a protein component of an arteriosclerotic plaque present in a biological sample obtained from a test subject are compared to a normal control value(s) or range of normal control values. The control value can be based on levels of the enzymatic cleavage product in comparable samples (e.g., blood, plasma, or serum sample, or other biological sample) obtained from a control population, e.g., the general population or a select population of human subjects. For example, the select population may be comprised of apparently healthy subjects, e.g., individuals who have not previously had any signs or symptoms of cardiovascular disease. Apparently healthy individuals also generally do not otherwise exhibit symptoms of disease. In other words, such individuals, if examined by a medical professional, would be characterized as healthy and free of symptoms of disease.

The control value can take a variety of forms. The control value can be a single cut-off value, such as a median or mean. A normal control value can be a normal control range. The control value can be established based on comparative groups such as where the risk in one defined group is double the risk in another defined group. The control values can be divided equally (or unequally) into groups, such as a low risk group, a medium risk group and a high-risk group, or into quadrants, the lowest quadrant being individuals with the lowest risk the highest quadrant being individuals with the highest risk, and the test subject's risk of having CVD can be based upon which group his or her test value falls.

### Biological Samples

Suitable biological samples useful for predicting or monitoring cardiovascular disease in a subject or for assessing the effect of therapeutic agents on subjects with cardiovascular disease include, but are not limited to, whole blood samples, and blood fractions (also referred to as “blood products”), where suitable blood fractions include, but are not limited to, serum and plasma. The biological sample can be fresh blood or stored blood (e.g. in a blood bank) or blood fractions. The biological sample can be a blood sample expressly obtained for an assay of the present disclosure or a blood sample obtained for another purpose which can be subsampled for an assay of the present disclosure. A suitable biological sample can also be a biological sample obtained via catheter during or as a result of coronary angiogram. A suitable biological sample can also be a biological sample obtained during catheterization of a carotid artery.

In one embodiment, the biological sample is whole blood. Whole blood can be obtained from the subject using standard clinical procedures. In another embodiment, the biological sample is plasma. Plasma can be obtained from whole blood samples by centrifugation of anti-coagulated blood. Such process provides a buffy coat of white cell components and a supernatant of the plasma. In another embodiment, the biological sample is serum.

The sample can be pretreated as necessary by dilution in an appropriate buffer solution, heparinized, concentrated if desired, or fractionated by any number of methods including but not limited to ultracentrifugation, fractionation by fast performance liquid chromatography (FPLC), or precipitation. Any of a number of standard aqueous buffer solutions, employing one of a variety of buffers, such as phosphate, Tris, or the like, at physiological pH can be used.

### Subjects

Subjects to be tested using a method of the present disclosure include individuals who have experienced one or more typical symptoms of cardiovascular disease; individuals who have experienced an atypical symptom of cardiovascular disease; smokers; non-smokers; individuals who have a body mass index greater than about 25 kg/m², or greater than about 30 kg/m²; individuals aged 50 years or older; and apparently healthy individuals. An individual can be male or female. In some instances, the individual is a female who has experienced an atypical symptom of cardiovascular disease, e.g., a symptom not normally associated with cardiovascular disease. In some instances, the individual has a disorder or disease involving a pathological process that pre-disposes the individual to a vascular occlusive event, where such pre-disposing diseases include, but are not limited to, systemic lupus erythematosus. In some instances, the individual does not have a history of having an occlusive vascular event.

In some cases, the individual has a disorder of lipid metabolism that can lead to plaque formation, where such disorders include, e.g., familial hypercholesterolemia; familial combined hyperlipidemia; high-density lipoprotein (HDL) deficiency; and other primary and secondary causes of dyslipidemia.

### Methods of Assessing Risk of an Occlusive Vascular Event

The present disclosure provides methods of determining a risk that an individual will develop an occlusive vascular event. The methods generally involve determining the level, in a biological sample from the individual, of an in vivo-generated enzymatic cleavage product of a protein component of an arteriosclerotic plaque. A level of the enzymatic cleavage product that is higher than a normal control level indicates risk of developing or experiencing an occlusive vascular event.

In some embodiments, a subject method for determining a risk that an individual will develop an occlusive vascular event comprises: a) assaying the level, in a biological sample from the individual, of an enzymatic cleavage product of a protein component of an arteriosclerotic plaque; and b) identifying the individual as being at risk of developing an occlusive vascular event when the level of the enzymatic cleavage product is higher than a normal control level. In some cases, as discussed below, a subject method for determining a risk that an individual will develop an occlusive vascular event further comprises outputting a report indicating a risk assessment based on said identifying to facilitate a treatment decision by a clinician.

Individuals to be subjected to a risk assessment method of the present disclosure include individuals who have experienced one or more typical symptoms of cardiovascular disease; individuals who have experienced an atypical symptom of cardiovascular disease; smokers; non-smokers; individuals who have a body mass index greater than about 25 kg/m², or greater than about 30 kg/m²; individuals aged 50 years and older; and apparently healthy individuals. An individual can be male or female. In some instances, the individual is a female who has experienced an atypical symptom of cardiovascular disease, e.g., a symptom not normally associated with cardiovascular disease. In some instances, the individual is not otherwise known to be at an elevated risk of having an occlusive vascular event. In some instances, the individual does not have a history of having an occlusive vascular event. In some instances, the individual has a disease or disorder that predisposes the individual to having an occlusive vascular event. In some cases, the individual has a disorder of lipid metabolism that can lead to plaque formation, where such disorders include, e.g., familial hypercholesterolemia; familial combined hyperlipidemia; high-density lipoprotein (HDL) deficiency; and other primary and secondary causes of dyslipidemia.

Suitable biological samples are as described above, and include, but are not limited to, blood; a blood product, such as serum or plasma; a biological sample obtained from a catheter during or as a result of coronary angiogram; and biological samples obtained during catheterization of a carotid artery.

Enzymatic cleavage products of a protein component of an arteriosclerotic plaque, and suitable methods of detecting same, are as described above. Suitable normal controls are as described above.

Occlusive vascular disease events include, but are not limited to, peripheral artery disease, arterial thrombosis, arterial occlusion, occlusive coronary arteriosclerosis, etc.

### Generating a Report

In some embodiments, a subject method of determining a risk that an individual will develop an occlusive vascular event further involves generating a report. Such a report can include information such as a predicted risk that the patient will experience an occlusive vascular event; a recommendation regarding further evaluation; a recommendation regarding therapeutic drug and/or device intervention; and the like.

For example, the methods disclosed herein can further include a step of generating or outputting a report providing the results of a subject risk assessment, which report can be provided in the form of an electronic medium (e.g., an electronic display on a computer monitor), or in the form of a tangible medium (e.g., a report printed on paper or other tangible medium). An assessment as to the likelihood can be referred to as a “risk report” or, simply, “risk score.” A person or entity that prepares a report (“report generator”) may also perform steps such as sample gathering, sample processing, and the like. Alternatively, an entity other than the report generator can perform steps such as sample gathering, sample processing, and the like. A risk assessment report can be provided to a user. A “user” can be a health professional (e.g., a clinician, a laboratory technician, a physician (e.g., a cardiologist), etc.).

### Further Evaluation

Based on detection an enzymatic cleavage product of a protein component of an arteriosclerotic plaque, and/or based on a report (as described above), a physician or other qualified medical personnel can determine whether further evaluation of the test subject (the patient) is required. Further evaluation can include, e.g., angiogram; electrocardiogram; an echocardiogram; a test for blood (or blood product, such as plasma or serum) triglyceride levels; a test for blood (or blood product, such as plasma or serum) low density lipoprotein levels; a test for blood (or blood product, such as plasma or serum) high density lipoprotein levels; and the like.

### Therapeutic Intervention

Based on detection of an enzymatic cleavage product of a protein component of an arteriosclerotic plaque, and/or based on a report (as described above), a physician or other qualified medical personnel can determine whether appropriate therapeutic intervention is advised, e.g., in order to reduce the risk that an occlusive vascular event will actually occur. Thus, in some embodiments, a subject method comprises detecting an unstable arteriosclerotic plaque in an individual (where the detection method comprises detecting in a biological sample from the individual an enzymatic cleavage product of a protein component of an arteriosclerotic plaque); and administering to the individual a therapeutic intervention for reducing an arteriosclerotic plaque.

Therapeutic intervention includes drug-based therapeutic intervention, device-based therapeutic intervention, and surgical intervention. Drug-based therapeutic intervention includes, an anti-inflammatory agent, an antithrombotic agent, an anti-platelet agent, a fibrinolytic agent, a lipid reducing agent, a direct thrombin inhibitor, a glycoprotein IIb/IIIa receptor inhibitor, an agent that binds to cellular adhesion molecules and inhibits the ability of white blood cells to attach to such molecules, a calcium channel blocker, a beta-adrenergic receptor blocker, a cyclooxygenase-2 inhibitor, and an angiotensin system inhibitor.

Device-based therapeutic intervention includes, e.g., installation of an intravascular stent; balloon angioplasty; and the like. Surgical intervention includes, e.g., arterial bypass surgery.

### Kits and Assay Devices

The present disclosure provides a kit for carrying out a method of the present disclosure, e.g., a method of detecting, in a biological sample obtained from an individual, an enzymatic cleavage product of a protein component of an arteriosclerotic plaque. The present disclosure further provides an assay device for carrying out a method of the present disclosure, e.g., a method of detecting, in a biological sample obtained from an individual, an enzymatic cleavage product of a protein component of an arteriosclerotic plaque.

### Kits

A subject kit can include: a) a binding reagent that specifically binds an enzymatic cleavage product of a protein component of an arteriosclerotic plaque; and b) a control that provides for quantitation of the enzymatic cleavage product.

In some embodiments, a subject kit includes standard enzymatic cleavage products of a protein component of an arteriosclerotic plaque, where the enzymatic cleavage product is of greater than 90% purity, greater than 95% purity, greater than 98% purity, or greater than 99% purity. The standard enzymatic cleavage product can be prepared synthetically, e.g., by incubating a protein component of an arteriosclerotic plaque with an enzyme (e.g., an enzyme that is secreted by an inflammatory cell, as described above); and isolating a fragment that corresponds to a fragment that is produced by an unstable arteriosclerotic plaque. The standard enzymatic cleavage product can be prepared synthetically, e.g., using standard chemical methods for peptide synthesis. Thus, in some embodiments, a subject kit includes purified enzymatic cleavage products of a protein component of an arteriosclerotic plaque, where the purified enzymatic cleavage products are suitable for generating a standard curve, e.g., for quantitating an enzymatic cleavage product of a protein component of an arteriosclerotic plaque detected in a test biological sample from a test individual.

In some embodiments, a subject kit includes a panel of purified enzymatic cleavage products of a protein component of an arteriosclerotic plaque, where the purified enzymatic cleavage products are suitable for generating a standard curve, e.g., for quantitating an enzymatic cleavage product of a protein component of an arteriosclerotic plaque detected in a test biological sample from a test individual.

A panel of enzymatic cleavage products (enzymatic cleavage products generated by an enzyme produced by a cell (e.g., an inflammatory cell) in the vasculature) suitable for inclusion in a subject kit can include 2, 3, 4, 5, 6, 7, 8, 9, 10, 10-15, 15-20, 20-25, 25-30, 30-35, 35-40, 40-45, 45-50, or more than 50, of the above-described enzymatic cleavage products.

As one non-limiting example, a suitable peptide panel includes:

1) one or more fibrillin fragments selected from:

(SEQ ID NO: 277)
ACEDIDECSLPNICVFGTCHNLPGLFR;
(SEQ ID NO: 278)
TGLPVDIDECR;
(SEQ ID NO: 279)
PVDIDECR;
(SEQ ID NO: 280)
EIPGVCNGVCINHVGSFR;
(SEQ ID NO: 281)
EIPGVCENGVCINMVGSFR;
(SEQ ID NO: 282)
LLVCEDIDECQNGPVCQR;
(SEQ ID NO: 283)
TCVDINECLLEPR;
(SEQ ID NO: 284)
GEGWGDPCELCPTEPDEAFR;
and

and naturally-occurring variants thereof;

2) one or more vitronectin fragments selected from:

(SEQ ID NO: 285)
CTDYTAECKPQVTR;
(SEQ ID NO: 286)
IYISGMAPRPS;
(SEQ ID NO: 287)
TCEPIQSVFFFSGDK;
(SEQ ID NO: 288)
SIAQYWLGCPAPGH;
and
(SEQ ID NO: 289)
WLGCPAPGHL,

and naturally-occurring variants of any of the foregoing;

3) one or more fibronectin fragments selected from:

(SEQ ID NO: 247)
GPGLLLLAVQCLGTAVPSTGASK;
(SEQ ID NO: 248)
ALVCTCYGGSR;
(SEQ ID NO: 327)
ISCTIANR;
(SEQ ID NO: 249)
MVDCTCLGEGSGR;
(SEQ ID NO: 250)
AAHEEICTTNEGVMYR;
(SEQ ID NO: 251)
SHPIQWNAPQPSHISK;
(SEQ ID NO: 252)
VVSWVSASDTVSGFR;
(SEQ ID NO: 253)
SDTVPSPRDLQFVEVTDVK;
(SEQ ID NO: 254)
VDVIPVNLPGEHGQR;
(SEQ ID NO: 255)
VFAVSHGRESKPLTAQQTTK;
(SEQ ID NO: 256)
LGVRPSQGGEAPR;
(SEQ ID NO: 257)
DAPIVNKVVTPLSPPTNLH;
(SEQ ID NO: 258)
TPDITGYR;
(SEQ ID NO: 259)
PGTEYVVSVSSVYEQHESTPLR;
(SEQ ID NO: 260)
TGLDSPTGIDFSDITANSFTVH;
(SEQ ID NO: 261)
TVHWIAPR;
(SEQ ID NO: 262)
SPVQEFTVPGSK;
(SEQ ID NO: 263)
VVSVYAQNPSGESQPLVQTAVTNIDRPK;
(SEQ ID NO: 264)
RPGSEYTVSVVALHDDMESQPLIGTQSTAIPAPTDLK;
(SEQ ID NO: 265)
YEVSVYALK;
(SEQ ID NO: 266)
IYLYTLNDNAR;
(SEQ ID NO: 267)
SLLVSWQPPR;
(SEQ ID NO: 268)
YEKPGSPPR;
(SEQ ID NO: 269)
TPFVTHPG;
(SEQ ID NO: 270)
TPFVTHPGYDT;
(SEQ ID NO: 271)
TPFVTHPGYDTGNGIQLPGTSGQQPSVGQQM;
(SEQ ID NO: 272)
QDTSEYIISCHPVGTDEEPLQFR;
(SEQ ID NO: 273)
VPGTSTSATLTGLTRGATYNIIVEALK;
(SEQ ID NO: 274)
VREEVVTVGN;
(SEQ ID NO: 275)
SVNEGLNQPTDDSCFDPYTVSHYAVGDEWER;
and
(SEQ ID NO: 276)
LGFGSGHFR,

and naturally-occurring variants of any of the foregoing.

A subject panel can further include:

4) one or more tenascin peptides selected from:

(SEQ ID NO: 318)
ELEPGVEYFIR;
(SEQ ID NO: 319)
TVSIYGVIQGYR;
(SEQ ID NO: 320)
TVTLHGEVR;
and
(SEQ ID NO: 321)
FRITYVPITGGTPSMVTVDGTK,

and naturally-occurring variants of any of the foregoing;

5) one or more prolargin peptides selected from:

(SEQ ID NO: 307)
EVPSALPR;
(SEQ ID NO: 308)
RLSQNHISR;
(SEQ ID NO: 309)
RLSQNHISRIPPGVFSK;
(SEQ ID NO: 310)
LSDGVFKPDT;
(SEQ ID NO: 311)
NLAHNILR;
(SEQ ID NO: 312)
LAHNILR;
(SEQ ID NO: 313)
LDSNKIETIPNGYFKSFPNLAFIR;
(SEQ ID NO: 314)
IETIPNGYFKSFPNLA;
(SEQ ID NO: 315)
SFPNLAFIRLNYN;
(SEQ ID NO: 316)
LNNNSIEK;
and
(SEQ ID NO: 317)
DLVAFHDFSSDLENVPHLR,

and naturally-occurring variants of any of the foregoing; and

6) one or more dermatopontin peptides selected from:

(SEQ ID NO: 293)
SDDGWVNLNR;
(SEQ ID NO: 294)
SYQCPQGQVIVAVR;
(SEQ ID NO: 295)
SLGEPTECWWEEINR;
and
(SEQ ID NO: 296)
SNNGLVAGFQSR,

and naturally-occurring variants of any of the foregoing.

In some cases, the reagent that specifically binds an enzymatic cleavage product of a protein component of an arteriosclerotic plaque is an antibody. Suitable antibodies include monoclonal antibodies, and antigen-binding fragments (e.g., a Fv, scFv, Fab, F(ab′)2, or Fab′ fragment). Where the binding reagent is an antibody, the antibody can be immobilized on an insoluble support (e.g., a test strip, a well of a multi-well plate, beads, etc.). Where the binding reagent is an antibody, the antibody can comprise a detectable label. Where the antibody comprises a detectable label, a subject kit can include one or more reagents for developing the detectable label. A labeled antibody can comprise a label such as a chemiluminescent agent, a particulate label, a colorimetric agent, an energy transfer agent, an enzyme, a fluorescent agent, or a radioisotope.

In some cases, a subject kit includes a plurality of antibodies, where each member of the plurality is specific for a different enzymatic cleavage product of a protein component of an arteriosclerotic plaque. For example, the plurality of antibodies can include individual member antibodies, each of which is specific for a different enzymatic cleavage product of a protein component of an arteriosclerotic plaque present in a panel of such enzymatic cleavage products.

A suitable antibody includes an antibody specific for an epitope that is exposed upon cleavage of a protein component of an arteriosclerotic plaque, e.g., an epitope comprising the newly-formed carboxyl-terminus or amino-terminus of an enzymatic cleavage product of a protein component of an arteriosclerotic plaque. For example, an enzyme(s) produced by an inflammatory cell in the vasculature can proteolytically cleave a protein component of an arteriosclerotic plaque; and the cleavage product would then present epitope(s) (e.g., linear epitopes; conformational epitopes) not presented by the uncleaved protein component, where such epitopes could include the C-terminal amino acids of the cleavage product, or the N-terminal amino acids of the cleavage product.

Other optional components of the kit include: a buffer; a protease inhibitor; a detectable label; etc. The various components of the kit may be present in separate containers or certain compatible components may be pre-combined into a single container, as desired.

In addition to above-mentioned components, a subject kit can include instructions for using the components of the kit to practice a subject method. The instructions for practicing a subject method are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. compact disc-read only memory (CD-ROM), digital versatile disk (DVD), diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### Assay Device

The present disclosure further provides an assay device for use in detecting, in a liquid biological sample obtained from an individual, an enzymatic cleavage product of an arteriosclerotic plaque. The device can include a matrix defining an axial flow path. The matrix can comprise: i) a sample receiving zone at an upstream end of the flow path that receives the liquid sample; ii) one or more test zones positioned within the flow path and downstream from the sample receiving zone, each of the one or more test zones comprising an antibody specific for an enzymatic cleavage product of an arteriosclerotic plaque in each of the test zones, where each of the immobilized antibodies is capable of binding a different enzymatic cleavage product present in the liquid sample, to form an immobilized enzymatic cleavage product; and iii) one or more control zones positioned within the flow path and downstream from the sample receiving zone, where the one or more control zones can include positive and/or negative controls. The test zones and control zones can be positioned in an alternating format within the flow path beginning with a test zone positioned upstream of any control zone.

In using such an assay device, in some embodiments, a labeled antibody specific for an enzymatic cleavage product of an arteriosclerotic plaque can first be mixed with a liquid sample before the liquid sample is applied to the sample receiving zone of the device, where such mixing results in a labeled antibody/enzymatic cleavage product complex. In these embodiments, the liquid sample comprising the labeled antibody/enzymatic cleavage product complex is applied to the sample receiving zone of the assay device. The liquid sample flows along the device until the liquid sample reaches a test zone. Antibody present in the test zone binds an enzymatic cleavage product present in the labeled antibody/enzymatic cleavage product complex; and can then be detected.

The assay device can further include a label zone comprising a labeled antibody specific for an enzymatic cleavage product of an arteriosclerotic plaque, where the labeled antibody is capable of binding a cleavage product present in an immobilized cleavage product complex, to form a labeled enzymatic cleavage product complex, where the labeled antibody is mobilizable in the presence of liquid sample. In using such an assay device, a liquid sample comprising an enzymatic cleavage product of an arteriosclerotic plaque is applied to the sample receiving zone of the device; antibody present in the label zone binds the enzymatic cleavage product, forming labeled antibody/enzymatic cleavage product complex, which, like the labeled antibody, is mobilizable; and the labeled antibody/enzymatic cleavage product complex flows alone the device until the liquid sample reaches a test zone. Antibody present in the test zone binds an enzymatic cleavage product present in the labeled antibody/enzymatic cleavage product complex; and can then be detected.

The labeled antibody can comprise a label such as a chemiluminescent agent, a particulate label, a colorimetric agent, an energy transfer agent, an enzyme, a fluorescent agent, or a radioisotope.

Control zones include positive control zones and negative control zones.

The matrix is generally an insoluble support, where suitable insoluble supports include, but are not limited to, polyvinyl difluoride (PVDF), cellulose, nitrocellulose, nylon, and the like. The matrix can be flexible, or can be relatively inflexible. The matrix can be positioned within a housing comprising a support and optionally a cover, where the housing contains an application aperture and one or more observation ports. The assay device can be in any of a variety of formats, e.g., a test strip, a dipstick; etc.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Identification of Enzymatic Cleavage Products in Unstable Arteriosclerotic Plaques

Arteriosclerotic plaques that appeared unstable by visual inspection were incubated in buffer. Proteins released from the plaque into the buffer, including any enzymatic cleavage products, were isolated. To facilitate mass spectrometry analysis, the isolated enzymatic cleavage products were trypsinized. The trypsinization products were then subjected to mass spectrometry analysis.

Enzymatic cleavage products that were not solely the result of trypsin digestion (non-tryptic peptides) were identified. Examples of non-tryptic fragments of proteins present in the plaque are listed below. These represent in vivo-generated enzymatic cleavage products from the arteriosclerotic plaques. The peptides are listed in sequence order within each protein. Peptides denoted with an asterisk were detected in more than one protein.

Collagen alpha-1(I)chain fragments
(SEQ ID NO: 26)
TGGISVPGPMGPSGPR
(SEQ ID NO: 27)
GLPGPPGAPGPQG
(SEQ ID NO: 28)
GLPGPPGAPGPQGF
(SEQ ID NO: 29)
PGEPGEPGASGPMGPRGPPGPPGK
(SEQ ID NO: 30)
GASGPMGPRGPPGPPGK
(SEQ ID NO: 31)
KPGRPGERGPPGPQGAR
(SEQ ID NO: 32)
GPPGPQGARGLPGTAGLPGM
(SEQ ID NO: 33)
AGPQGPR
(SEQ ID NO: 34)
GAPGIAGAPGFPGAR
(SEQ ID NO: 35)
PGIAGAPGFPGARGPSGPQGPGGPPGPK
(SEQ ID NO: 36)
IAGAPGFPGARGPSGPQGPGGPPGPK
(SEQ ID NO: 37)
GFPGARGPSGPQGPGGPPGPK
(SEQ ID NO: 38)
GPSGPQGPGG
(SEQ ID NO: 39)
GDTGAKGEP
(SEQ ID NO: 40)
VQGPPGPAGEEGK
(SEQ ID NO: 41)
GEPGPTGLPGPPG
(SEQ ID NO: 42)
GEPGPTGLPGPPGERGGPGS
(SEQ ID NO: 43)
TGLPGPPGER
(SEQ ID NO: 44)
LPGPPGER
(SEQ ID NO: 45)
AGPKGPAGER
(SEQ ID NO: 46)
GSPGPAGPKGSPGEAGRPGEAG
(SEQ ID NO: 47)
PGEAGRPGEAGLPGAKGLTGSPGSPGPDGK
(SEQ ID NO: 48)
LTGSPGSPGPDGK
(SEQ ID NO: 49)
TGPPGPAGQDGRPGPPGPPGARG
(SEQ ID NO: 50)
PGAVGPAGKDGEAGAQGPPGPAGPAGER
(SEQ ID NO: 51)
GEAGAQGPPGPAGPAGER
(SEQ ID NO: 52)
EAGAQGPPGPAGPAGER
(SEQ ID NO: 53)
VQGPPGPAGPR
(SEQ ID NO: 54)
QGPPGPAGPR*
(SEQ ID NO: 55)
GPPGPAGPR
(SEQ ID NO: 56)
ANGAPGNDGAKGDAGAPGAPGSQGAPGLQGMPGER
(SEQ ID NO: 57)
LQGMPGER*
(SEQ ID NO: 58)
LTGPIGPPGPAGAPGDK
(SEQ ID NO: 59)
IGPPGPAGAPGDK
(SEQ ID NO: 60)
KGESGPSGPAGPTGAR
(SEQ ID NO: 61)
PGDRGEPGPPGPAGFAGPPGADGQPGAK
(SEQ ID NO: 62)
GEPGPPGPAGF
(SEQ ID NO: 63)
FAGPPGADGQPGAK*
(SEQ ID NO: 64)
AGPPGADGQPGAK*
(SEQ ID NO: 65)
RVGPPGPSGNAGPPGPPGPAGK
(SEQ ID NO: 66)
VGPPGPSGNAGPPGPPGPAGKEGG
(SEQ ID NO: 67)
EVGPPGPPGPAGEKGSPGADGPAGAPGTPGPQGIAGQR
(SEQ ID NO: 68)
PGPPGPAGEKGSPGADGPAGAPGTPGPQGIAGQR
(SEQ ID NO: 69)
GSPGADGPAGAPGTPGPQG
(SEQ ID NO: 70)
GPAGAPGTPGPQGIAGQR
(SEQ ID NO: 71)
VVGLPGQR
(SEQ ID NO: 72)
LAGPPGESGR
(SEQ ID NO: 73)
ETGPAGPPGAPGAPGAPGPVGPAGKSGDR
(SEQ ID NO: 74)
RGETGPAGPAGPVGPVGAR
(SEQ ID NO: 75)
PAGPVGPVGAR
(SEQ ID NO: 76)
PVGPVGAR
(SEQ ID NO: 77)
SPGEQGPSGASGPAGPR
(SEQ ID NO: 78)
PGEQGPSGASGPAGPR
(SEQ ID NO: 79)
GPSGASGPAGPR
(SEQ ID NO: 80)
ASGPAGPR
(SEQ ID NO: 81)
GPPGSAGAPGKD
(SEQ ID NO: 82)
PPGSAGAPGKDGLNGLPGPIGPPGPR
and
(SEQ ID NO: 83)
LP QPPQEK*
Collagen alpha-2(I)chain fragments
(SEQ ID NO: 84)
GLMGPRGPPGAAGAPGPQGFQGPAGEPGEPGQTGPAGAR
(SEQ ID NO: 85)
FQGPAGEPGEPGQTGPAGAR
(SEQ ID NO: 86)
QGPAGEPGEPGQTGPAGAR
(SEQ ID NO: 87)
EDGHPGKPGRPGERGVVGPQGAR
(SEQ ID NO: 88)
PAGARGSDGSVGPVGPAGPIGSAGPPGFPGAPGPK
(SEQ ID NO: 89)
DGSVGPVGPAGPIGSAGPPGFPGAPGPK
(SEQ ID NO: 90)
PGAPGPKGEIGAVGNAGPAGPAGPR
(SEQ ID NO: 91)
GPAGPAGPR
(SEQ ID NO: 92)
PAGPAGPR
(SEQ ID NO: 93)
RGEVGLPGLSGPVGPPGNPGANGLTGAK
(SEQ ID NO: 94)
GAPGLPGPR
(SEQ ID NO: 95)
PNGEAGSAGPPGPPGLR
(SEQ ID NO: 96)
GPRGLPGSPGNIGPAGK
(SEQ ID NO: 97)
GRPGPIGPAGAR
(SEQ ID NO: 98)
GPSGPPGPDG
(SEQ ID NO: 99)
GPSGPPGPDGNKGEPGVVGAVGTAGPS
(SEQ ID NO: 100)
GPSGLPGER
(SEQ ID NO: 101)
GAVGAPGPAGATGDRGEAGAAGPAGPAGPR
(SEQ ID NO: 102)
VGAPGPAGATGDRGEAGAAGPAGPAGPR
(SEQ ID NO: 103)
PGPAGATGDRGEAGAAGPAGPAGPR
(SEQ ID NO: 104)
NGVVGPTGPVGAAGPAGPNGPPGPAGSR
(SEQ ID NO: 105)
GPPGPAGSR
(SEQ ID NO: 106)
PGPAGSRGDGGPPGMTGFPGAAGR
(SEQ ID NO: 107)
GDGGPPGMTGFPGAAGRTGPPGPSGISGPPGPPGPA
(SEQ ID NO: 108)
ISGPPGPPGPAGK
(SEQ ID NO: 109)
GPSGEAGTAGPPGTPGPQGL
(SEQ ID NO: 110)
PGILGLPGSR
(SEQ ID NO: 111)
IAGPPGAR
(SEQ ID NO: 112)
PGNIGPVGAAGAPGPHGPVGPAGKHGNR
(SEQ ID NO: 113)
VGPAGAVGPR
(SEQ ID NO: 114)
QGAPGSVGPAGPR
(SEQ ID NO: 115)
GPAGPSGPAGKD
(SEQ ID NO: 116)
GTVGPAGIR
Collagen alpha-1(III)chain fragments
(SEQ ID NO: 117)
GPQGPKGDPGPPGIPGR
(SEQ ID NO: 118)
PGTSGHPGSPGSPGYQGPPGEPGQAGPSGPPGPPGAIGPSGPAGK
(SEQ ID NO: 119)
GLPGPPGIKGPAG
(SEQ ID NO: 120)
GEVGPAGSPGSNGAPGQRGEPGPQGHAG
(SEQ ID NO: 121)
GEPGPQGHAGAQGPPGPPGINGSPGGKGEMGPAGIPG
(SEQ ID NO: 122)
GEMGPAGIPGAPGLMGARGPPGPAG
(SEQ ID NO: 123)
GIPGAPGLMGAR
(SEQ ID NO: 124)
GAPGLMGARGPPGPAGANGAPGLR
(SEQ ID NO: 125)
PAGERGAPGPAGPR
(SEQ ID NO: 126)
GAPGPAGPRGAAGEP
(SEQ ID NO: 127)
GEPGRDGVPGGPGMR
(SEQ ID NO: 128)
DGKPGPPGSQGESGRPGPPGPSGPR
(SEQ ID NO: 129)
GKPGPPGSQGESGRPGPPGPSGPR
(SEQ ID NO: 130)
GRPGPPGPSGPR
(SEQ ID NO: 131)
GPPGPSGPR
(SEQ ID NO: 132)
QGPPGKNGETGPQGPPGPTGPGGDK
(SEQ ID NO: 133)
GDAGAPGERGP
(SEQ ID NO: 134)
LQGMPGER
(SEQ ID NO: 135)
GEGGPPGVAGPPGGSGPAGPPGPQGV
(SEQ ID NO: 136)
GSNGNPGPPGPSGSPGKDGPPGPAGNTGAPGSPGVSGPK
(SEQ ID NO: 137)
NGNPGPPGPSGSPGK
(SEQ ID NO: 138)
GSPGAQGPP
(SEQ ID NO: 139)
GNPGSDGLPGR
(SEQ ID NO: 140)
ENGSPGAPGAPGHPGPPGPVGPAGK
(SEQ ID NO: 141)
PGAPGAPGHPGPPGPVGPAGK
(SEQ ID NO: 142)
RGESGPAGPAGAPGPAGSR
(SEQ ID NO: 143)
GESGPAGPAGAP
(SEQ ID NO: 144)
PGAPGSPGPAGQQGAIGSPGPAGPR
(SEQ ID NO: 145)
GQQGAIGSPGPAGPRGPVGPSGPPGK
(SEQ ID NO: 146)
QGAIGSPGPAGPR
and
(SEQ ID NO: 147)
GSEGSPGHPGQPGPPGPPGAPGPCCGGVGAAAIAGIGGEKAGGFAPYYG
Collagen alpha-1(II)chain fragments
(SEQ ID NO: 148)
GPQGFQGNPGEPGEPGVSGPMGPRGPPGPPGKPGDDGEAGKPGK
(SEQ ID NO: 149)
GAAGARGNDGQPGPAGPPGPVGPAGGPGFPGAPGAK
(SEQ ID NO: 150)
AAGARGNDGQPGPAGPPGPVGPAGGPGFPGAPGAK
(SEQ ID NO: 151)
PGAKGSAGAPGIAGAPGFPGPR
(SEQ ID NO: 152)
GPRGPPGPQGATGPLGPK
(SEQ ID NO: 153)
DGLAGPK
(SEQ ID NO: 154)
PQGKVGPSGAPGEDGRPGPPGPQGAR
(SEQ ID NO: 155)
GFPGPKGANGEPGK
(SEQ ID NO: 156)
GLPGPPGPPGEGGKPGDQGVPGEAGAPGLVGPR
(SEQ ID NO: 157)
GPPGEGGKPGDQGVPGEAGAPGLVGPR
(SEQ ID NO: 158)
LQGMPGER
(SEQ ID NO: 159)
GRGLTGPIGPPGPAGANGEK
(SEQ ID NO: 160)
GLTGPIGPPGPAGANGEKGEVGPP
(SEQ ID NO: 161)
GLTGPIGPPGPAGANGEKGEVGPPGPAGSAG
(SEQ ID NO: 162)
LTGPIGPPGPAGANGEKGEVGPPGPAGSAGAR
(SEQ ID NO: 163)
TGPIGPPGPAGANGEKGEVGPPGPAGSAGAR
(SEQ ID NO: 164)
FAGPPGADGQPGAK*
(SEQ ID NO: 165)
AGPPGADGQPGAK*
(SEQ ID NO: 166)
SGPPGRAGEPGLQGPAGPPGEK
(SEQ ID NO: 167)
GPPGRAGEPGLQGPAGPPGEK
(SEQ ID NO: 168)
PPGLTGPAGEPGREGSPGADGPPGR
(SEQ ID NO: 169)
PGPGIDMSAFAGLGPREK
Collagen alpha-1(XIV)chain fragments
(SEQ ID NO: 170)
IEWHLNAF
(SEQ ID NO: 171)
AITGPPTELITSEVTAR
(SEQ ID NO: 172)
AIYAHTASEGLR
(SEQ ID NO: 173)
LYDVTENSMR
(SEQ ID NO: 174)
YLILYAPLTEGLAGDEKEMK
(SEQ ID NO: 175)
YAPLTEGLAGDEK
(SEQ ID NO: 176)
HVEMTSLCAH
(SEQ ID NO: 177)
SIQGMPGMPGEKGEK
(SEQ ID NO: 178)
QVCEQLIQSH
(SEQ ID NO: 179)
EPGRPGSPGAPGEQGPPGTPGFPGNAGVPGTPGER
Collagen alpha-1(XII)chain fragments
(SEQ ID NO: 180)
GGSTNTGKAMTYVRE
(SEQ ID NO: 181)
PKVMILITDGK
(SEQ ID NO: 182)
PDDTHAYNVADFESLSR
(SEQ ID NO: 183)
SVVEDEYSEPLK
(SEQ ID NO: 184)
SETSTSLKD
(SEQ ID NO: 185)
LKPDTPYTITVSSLYPDGEGGRMTG
(SEQ ID NO: 186)
PGPAGGPGAK
(SEQ ID NO: 187)
GRTGTPGLPGPPGPMGPPGDR
(SEQ ID NO: 188)
TPGLPGPPGPMGPPGDRGFTGK
(SEQ ID NO: 189)
GFPGTPGMQGPPGERGLPGEK
(SEQ ID NO: 190)
QGPPGER
(SEQ ID NO: 191)
PRGLPGPPGPQGESR
Collagen alpha-1(XVIII)chain fragments
(SEQ ID NO: 192)
PPSLGRPWAPLTGPSVPPPSSGR
(SEQ ID NO: 193)
PGEDGKPGDTGPQGFPGTPGDVGPKGDK
(SEQ ID NO: 194)
PGLPGEPGR
(SEQ ID NO: 195)
GREGPPGFPGLPGPPGPPGR
(SEQ ID NO: 196)
QDGSVLSVPGPEGRPGFAGFPGPAGPKGNLGSK
(SEQ ID NO: 197)
AESSRPGPPGLPGNQGPPGPK
(SEQ ID NO: 198)
GPPGPKGAK
(SEQ ID NO: 199)
PGPPGPPGTMGASSGVR
(SEQ ID NO: 200)
RLPEPQPYPGAPHHSSYVHLRPARPTSPPAHSHR
(SEQ ID NO: 201)
LPEPQPYPGAPHHSSY
(SEQ ID NO: 202)
NSPLSGGMR
(SEQ ID NO: 203)
PSLGRPWAPLTGPSVPPPSSER
Collagen alpha-2(IV)chain fragments
(SEQ ID NO: 204)
GARGVSGFPGADGIPGHPGQGGPR
(SEQ ID NO: 205)
GGPKGLPGLPGPPGPTGAK
(SEQ ID NO: 206)
GPPGLHGFPGAPGQEGPLGLPGIPGREGLPGDR
(SEQ ID NO: 207)
APGRPGSPGLPGMPGR
(SEQ ID NO: 208)
LYCNPGDVCYYASR
(SEQ ID NO: 209)
LMHTAAGDEGGGQSLVSPGSCLEDFR
Collagen alpha-1(IV)chain fragments
(SEQ ID NO: 210)
EPGPPGLPGSVGSPGVPGIGPPGAR
(SEQ ID NO: 211)
PGVPGIGPPGARGPPGGQGPPGLSGPPGIK
(SEQ ID NO: 212)
PPGGQGPPGLSGPPGIKGEK
(SEQ ID NO: 213)
DPGFQGMPGIGGSPGITGSK
(SEQ ID NO: 214)
KGQQGVTGLVGIPGPPGIPGFDGAPGQK
(SEQ ID NO: 215)
SLLYVQGNER
(SEQ ID NO: 216)
LFCNINNVCNFASR
(SEQ ID NO: 217)
VMHTSAGAEGSGQALASPGSCLEEFR
(SEQ ID NO: 218)
RSAPFIECHGR
(SEQ ID NO: 219)
SFWLATIER
(SEQ ID NO: 220)
WLATIER
Collagen alpha-5(IV)chain fragments
(SEQ ID NO: 221)
DGIPGPPGPK
(SEQ ID NO: 222)
KGNPGYPGPPGIQGLPGPTGIPGPIGPPGPPGLMGPPGPPGLPGPK
(SEQ ID NO: 223)
PHIPPSDEICEPGPPGPPGSPGDK
(SEQ ID NO: 224)
GLPGLPGPPGSLGFPGQK
(SEQ ID NO: 225)
PKGEPGGITFK
(SEQ ID NO: 226)
TPGRIGLEGPPGPPGFPGPK
(SEQ ID NO: 227)
GPPGRTGLDGLPGPK
(SEQ ID NO: 228)
APGPIGPPGSPGLPGK
(SEQ ID NO: 229)
KGEPGLPGPPGPMDPNLLGSK
(SEQ ID NO: 230)
PGEPGPVGGGGHPGQPGPPGEK
(SEQ ID NO: 231)
PALEGPKGNPGPQGPPGRPGPTGFQGLPGPEGPPGLPGNGGIK
(SEQ ID NO: 232)
GPPGPPGLPGPSGQSIIIK
Collagen alpha-1(XV)chain fragments
(SEQ ID NO: 233)
VDGATGLPGMK
(SEQ ID NO: 234)
KGQAGPPGVMGPPGPPGPPGPPGPGCTMGLGFED
(SEQ ID NO: 235)
KLQLGELIPIPADSPPPP
(SEQ ID NO: 236)
AWRTADTAVTGLASPLSTGK
(SEQ ID NO: 237)
AVTGLASPLSTGKILDQK
Collagen alpha-3(VI)chain fragments
(SEQ ID NO: 238)
GVEDADEGALKEIASEPLNMHMFNLENFTSLHDIVGNLVSCVHSSVSPER
(SEQ ID NO: 241)
IGDLHPQIVN
Collagen alpha-1(VI)chain fragments
(SEQ ID NO: 242)
GPQGDQGR
(SEQ ID NO: 244)
FSDGNSQGATPAAIEK
Collagen alpha-1(XIX)chain fragment
(SEQ ID NO: 355)
NPGAPGPR
Fibronectin fragments
(SEQ ID NO: 247)
GPGLLLLAVQCLGTAVPSTGASK
(SEQ ID NO: 248)
ALVCTCYGGSR
(SEQ ID NO: 327)
ISCTIANR
(SEQ ID NO: 249)
MVDCTCLGEGSGR
(SEQ ID NO: 250)
AAHEEICTTNEGVMYR
(SEQ ID NO: 251)
SHPIQWNAPQPSHISK
(SEQ ID NO: 252)
VVSWVSASDTVSGFR
(SEQ ID NO: 253)
SDTVPSPRDLQFVEVTDVK
(SEQ ID NO: 254)
VDVIPVNLPGEHGQR
(SEQ ID NO: 255)
VFAVSHGRESKPLTAQQTTK
(SEQ ID NO: 256)
LGVRPSQGGEAPR
(SEQ ID NO: 257)
DAPIVNKVVTPLSPPTNLH
(SEQ ID NO: 258)
TPDITGYR
(SEQ ID NO: 259)
PGTEYVVSVSSVYEQHESTPLR
(SEQ ID NO: 260)
TGLDSPTGIDFSDITANSFTVH
(SEQ ID NO: 261)
TVHWIAPR
(SEQ ID NO: 262)
SPVQEFTVPGSK
(SEQ ID NO: 263)
VVSVYAQNPSGESQPLVQTAVTNIDRPK
(SEQ ID NO: 264)
RPGSEYTVSVVALHDDMESQPLIGTQSTAIPAPTDLK
(SEQ ID NO: 265)
YEVSVYALK
(SEQ ID NO: 266)
IYLYTLNDNAR
(SEQ ID NO: 267)
SLLVSWQPPR
(SEQ ID NO: 268)
YEKPGSPPR
(SEQ ID NO: 269)
TPFVTHPG
(SEQ ID NO: 270)
TPFVTHPGYDT
(SEQ ID NO: 271)
TPFVTHPGYDTGNGIQLPGTSGQQPSVGQQM
(SEQ ID NO: 272)
QDTSEYIISCHPVGTDEEPLQFR
(SEQ ID NO: 273)
VPGTSTSATLTGLTRGATYNIIVEALK
(SEQ ID NO: 274)
VREEVVTVGN
(SEQ ID NO: 275)
SVNEGLNQPTDDSCFDPYTVSHYAVGDEWER
(SEQ ID NO: 276)
LGFGSGHFR
Fibrillin fragments
(SEQ ID NO: 278)
TGLPVDIDECR
(SEQ ID NO: 279)
PVDIDECR
Vitronectin fragments
(SEQ ID NO: 285)
CTDYTAECKPQVTR
(SEQ ID NO: 286)
IYISGMAPRPS
(SEQ ID NO: 287)
TCEPIQSVFFFSGDK
(SEQ ID NO: 288)
SIAQYWLGCPAPGH
(SEQ ID NO: 289)
WLGCPAPGHL
Metalloproteinase inhibitor 1 fragments
(SEQ ID NO: 290)
CNSDLVIR
(SEQ ID NO: 291)
LQDGLLHITTC
(SEQ ID NO: 292)
SFVAPWNSLSLAQR
Dermatopontin fragments
(SEQ ID NO: 293)
SDDGWVNLNR
(SEQ ID NO: 294)
SYQCPQGQVIVAVR
(SEQ ID NO: 295)
SLGEPTECWWEEINR
(SEQ ID NO: 296)
SNNGLVAGFQSR
Galectin-1 fragments
(SEQ ID NO: 297)
VASNLNLKPGECLR
(SEQ ID NO: 298)
GDANTIVCNSK
(SEQ ID NO: 299)
MAADGDFK
Lumican fragments
(SEQ ID NO: 300)
CAPECNCPESYPSAMYCDELK
(SEQ ID NO: 301)
RNNQIDHIDEK
(SEQ ID NO: 302)
NNQIDHIDE
(SEQ ID NO: 303)
ILDHNLLENSK
(SEQ ID NO: 304)
SLEDLQLTH
(SEQ ID NO: 305)
IHLQHNR
(SEQ ID NO: 306)
CKILGPLSYSK
Prolargin fragments
(SEQ ID NO: 307)
EVPSALPR
(SEQ ID NO: 308)
RLSQNHISR
(SEQ ID NO: 309)
RLSQNHISRIPPGVFSK
(SEQ ID NO: 310)
LSDGVFKPDT
(SEQ ID NO: 311)
NLAHNILR
(SEQ ID NO: 312)
LAHNILR
(SEQ ID NO: 313)
LDSNKIETIPNGYFKSFPNLAFIR
(SEQ ID NO: 314)
IETIPNGYFKSFPNLA
(SEQ ID NO: 315)
SFPNLAFIRLNYN
(SEQ ID NO: 316)
LNNNSIEK
(SEQ ID NO: 317)
DLVAFHDFSSDLENVPHLR
Tenascin fragments
(SEQ ID NO: 318)
ELEPGVEYFIR
(SEQ ID NO: 319)
TVSIYGVIQGYR
(SEQ ID NO: 320)
TVTLHGEVR
(SEQ ID NO: 321)
FRITYVPITGGTPSMVTVDGTK
Tenascin-X fragments
(SEQ ID NO: 322)
WRPQPPAEGPGGELTVPGTTRTVS
(SEQ ID NO: 323)
FDSFTVQYK
(SEQ ID NO: 324)
GEESEVTVGGLEPGR
(SEQ ID NO: 325)
EPPNKPR
(SEQ ID NO: 326)
GFEESEPLTGFLTTVPDGPTQ.

### Example 2

### Detecting Enzymatic Cleavage Products of an Arteriosclerotic Plaque

Blood is drawn into tubes containing buffered ethylenediamine tetraacetic acid (EDTA). Plasma is separated by centrifugation. Target protein fragments are monomerized by addition of 0.005% Tween 20 in 0.005 M phosphate buffer, pH 7.4.

Target peptide fragments are quantitated by ELISA using monovalent antibodies. Synthetic peptides are used as standards. The contents of target peptides are reported in mass units per mL.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method for detecting an unstable arteriosclerotic plaque in an individual, the method comprising detecting in a biological sample from the individual an enzymatic cleavage product of a protein component of an arteriosclerotic plaque.

2. The method of claim 1, wherein the protein component is a structural protein.

3. The method of claim 1, wherein the protein component is a non-enzymatic protein.

4. The method of claim 1, wherein the individual is asymptomatic with respect to an arterial occlusive event.

5. The method of claim 1, wherein the subject is an apparently healthy human subject.

6. The method of claim 1, wherein the individual has experienced one or more typical symptoms of cardiovascular disease.

7. The method of claim 1, wherein the individual has experienced an atypical symptom of cardiovascular disease.

8. The method of claim 1, wherein the biological sample is blood or a blood fraction.

9. The method of claim 6, wherein the blood fraction is serum or plasma.

10. The method of claim 1, wherein the level of the one or more enzymatic cleavage products is determined by an immunological method.

11. The method of claim 1, wherein the protein component is fibrillin, vitronectin, fibronectin, tenascin, prolargin, dermatopontin, vascular collagen, metalloproteinase inhibitor-1, galectin-1, or tenascin-X.

12. The method of claim 11, where the collagen is collagen alpha-1 (I) chain, collagen alpha-1 (II) chain, collagen alpha-1 (IV) chain, collagen alpha-1 (VI) chain, collagen alpha-1 (XII), collagen alpha-1 (XIV) chain, collagen alpha-1 (XV) chain, collagen alpha-1 (XVIII), collagen alpha-1 (XIX), collagen alpha-2 (I) chain, collagen alpha-3 (VI), collagen alpha-2 (IV), or collagen alpha-5 (IV).

13. The method of claim 1, wherein the enzymatic cleavage product has a molecular weight in a range of from about 0.5 kDa to about 50 kDa.

14. The method of claim 1, wherein the enzymatic cleavage product has a length in a range of from about 5 amino acids to about 500 amino acids.

15. The method of claim 1, wherein said detecting further comprises processing the enzymatic cleavage product in vitro.

16. The method of claim 15, wherein said processing comprises trypsin digestion.

17. The method of claim 1, wherein the enzymatic cleavage product is a cleavage product of a matrix metalloproteinase (MMP).

18. The method of claim 17, wherein the MMP is secreted by a macrophage.

19. The method of claim 17, wherein the MMP is MMP1, MMP2, MMP3, MMP7, MMP8, MMP9, MMP10, MMP11, MMP12, or MMP13.

20. The method of claim 1, wherein the enzymatic cleavage product is a cleavage product of a cathepsin.

21. The method of claim 1, wherein the method comprises generating a report providing an indication of the risk that the individual will experience an occlusive vascular event.

22. A method for determining a risk that an individual will develop an occlusive vascular event, the method comprising:
a) assaying the level, in a biological sample from the individual, of an enzymatic cleavage product of a protein component of an arteriosclerotic plaque;
b) identifying the individual as being at risk of developing an occlusive vascular event when the level of the enzymatic cleavage product is higher than a normal control level.

23. The method of claim 22, wherein the protein component is fibrillin, vitronectin, fibronectin, tenascin, prolargin, dermatopontin, vascular collagen, metalloproteinase inhibitor-1, galectin-1, or tenascin-X.

24. The method of claim 22, wherein the level of the one or more enzymatic cleavage products is determined by an immunological method.

25. The method of claim 22, wherein the biological sample is blood, serum, or plasma.

26. The method of claim 22, wherein the subject is an apparently healthy human subject.

27. The method of claim 22, wherein the individual does not have a history of having an occlusive vascular event.

28. The method of claim 22, further comprising outputting a report indicating a risk assessment based on said identifying to facilitate a treatment decision by a clinician.

29. A kit for detecting an unstable arteriosclerotic plaque in an individual, the kit comprising:
a) a binding reagent that specifically binds an enzymatic cleavage product of a protein component of an arteriosclerotic plaque;
b) a control that provides for quantitation of the enzymatic product.

30. The kit of claim 29, wherein the reagent that specifically binds an enzymatic cleavage product of a protein component of an arteriosclerotic plaque is an antibody.

31. The kit of claim 30, wherein the antibody is a monoclonal antibody, or an antigen-binding fragment.

32. The kit of claim 29, wherein the antibody is immobilized on an insoluble support.

33. The kit of claim 29, wherein the antibody comprises a detectable label.

34. The kit of claim 29, further comprising one or more reagents for developing a detectable label.

35. An assay device for use in detecting, in a liquid biological sample obtained from an individual, an enzymatic cleavage product of a protein component of an arteriosclerotic plaque, the device comprising a matrix defining an axial flow path, the matrix comprising:
i) a sample receiving zone at an upstream end of the flow path that receives the liquid sample;
ii) one or more test zones positioned within the flow path and downstream from the sample receiving zone, each of said one or more test zones comprising an antibody specific for an enzymatic cleavage product of a protein component of an arteriosclerotic plaque immobilized in each of said test zones, wherein each of said immobilized antibodies is capable of binding different enzymatic cleavage product present in said liquid sample, to form an immobilized antibody/enzymatic cleavage product complex; and
iii) one or more control zones positioned within the flow path and downstream from the sample receiving zone.

36. The assay device of claim 35, wherein the one or more control zones are positioned between the test zones when two or more test zones are present.

37. The assay device of claim 35, wherein the test zones and control zones are positioned in an alternating format within the flow path beginning with a test zone positioned upstream of any control zone.

38. The assay device of claim 35, further comprising a label zone positioned upstream of a test zone, wherein the label zone comprises a labeled antibody specific for an enzymatic cleavage product of a protein component of an arteriosclerotic plaque, wherein the labeled antibody is capable of binding an enzymatic cleavage product present in an immobilized antibody/enzymatic cleavage product complex to form a labeled immobilized antibody/enzymatic cleavage product complex, and wherein the labeled antibody is mobilizable in the presence of the liquid sample.

39. The assay device of claim 38, wherein the labeled antibody comprises a label component selected from the group consisting of a chemiluminescent agent, a particulate label, a colorimetric agent, an energy transfer agent, an enzyme, a fluorescent agent, and a radioisotope.

40. The assay device of claim 35, wherein the matrix is positioned within a housing comprising a support and optionally a cover, wherein the housing contains an application aperture and one or more observation ports.

41. The assay device of claim 35, wherein the device is a test strip.

42. The assay device of claim 35, wherein the device is a dipstick assay device.

43. The assay device of claim 35, wherein the liquid sample is blood, serum, or plasma.

44. A panel of purified enzymatic cleavage products of a protein component of an arteriosclerotic plaque.

45. The panel of claim 44, wherein the panel comprises 2, 3, 4, 5, 6, 7, 8, 9, 10, 10-15, 15-20, 20-25, 25-30, 30-35, 35-40, 40-45, 45-50, or more than 50, different enzymatic cleavage products.

46. The panel of claim 44, wherein the protein component is fibrillin, vitronectin, fibronectin, tenascin, prolargin, dermatopontin, vascular collagen, metalloproteinase inhibitor-1, galectin-1, or tenascin-X.

47. The panel of claim 44, wherein each enzymatic cleavage product has a length in a range of from about 5 amino acids to about 500 amino acids.
